(19) United States
(12) Patent Application Publication
(10) Pub. No.: US 2009/0208492 A1
(43) Pub. Date: Aug. 20, 2009

(54) Lyophilized Immunoglobulin Formulations and Methods of Preparation

(75) Inventors: Barbara Horsey O'Connor, San Carlos, CA (US); Shaun E. Buckley, San Francisco, CA (US); David Burke, Oakland, CA (US)

Correspondence Address: CROWELL & MORING LLP;INTELLECTUAL PROPERTY GROUP, P.O. BOX 14300, WASHINGTON, DC, 20044-4300, US

(73) Assignee: Elan Pharmaceuticals, Inc., South San Francisco, CA (US)

(21) Appl. No.: 12/138,075

(22) Filed: Jun. 12, 2008

Related U.S. Application Data
(60) Provisional application No. 60/929,133, filed on Jun. 14, 2007.

(51) Int. Cl. A61K 39/395

(52) U.S. Cl. 424/133.1

(57) ABSTRACT

The present invention relates generally to the field of pharmaceutical formulation of immunoglobulins. Specifically, the present invention relates to stable, lyophilized, high concentration immunoglobulin formulations. This invention is exemplified by a stabilized lyophilized formulation of the recombinant humanized anti-alpha-4 integrin antibody natalizumab.

### CROSS REFERENCE TO RELATED APPLICATIONS

This application claims priority under 35 U.S.C. § 119 to U.S. Provisional Application No. 60/929,133, filed Jun. 14, 2007, the entire content of which is hereby expressly incorporated by reference in its entirety.

The present invention relates generally to the field of pharmaceutical formulation of immunoglobulins. Specifically, the present invention relates to stable, lyophilized, high concentration immunoglobulin formulations. This invention is exemplified by a stabilized lyophilized formulation of the recombinant humanized anti-alpha-4 integrin antibody natalizumab.

### BACKGROUND OF THE INVENTION

Drug preparations intended for administration to humans may require stabilizers to prevent alternations to the drugs prior to the use of the preparation. Because proteins are larger and more complex than traditional organic and inorganic drugs (i.e. proteins possess multiple functional groups in addition to complex three-dimensional structures), the formulation of proteins poses special problems. Degradation pathways for proteins can involve chemical instability (any process which involves modification of the protein by bond formation or cleavage resulting in a new chemical entity) or physical instability (changes in the higher order structure of the protein). Chemical instability can result from deamidation, racemization, hydrolysis, oxidation, beta elimination or disulfide exchange. Physical instability can result from denaturation, aggregation, precipitation or adsorption, for example. Many protein preparations are particularly unstable in very dilute or highly concentrated solutions, and this instability is often increased when the protein preparation is stored, or shipped. Thus, a major challenge that exists in the field of protein drugs is in the development of formulations that maintain both protein stability and activity.

Antibodies, including monoclonal antibodies, all differ from one another in ways relevant to their behavior and efficacy in a formulation. For example, monoclonal antibodies differ from one another with regard to isoelectric points, solubility, and conditions at which the monoclonal antibodies will aggregate. Proteins differ from one another with regard to their behavior and efficacy in a formulation, making it difficult to predict if a formulation will be stable for a particular antibody. Three common problems in protein formulations include protein degradation, aggregation, deamidation, and oxidation. Further, many different reactions affecting formulation stability may occur simultaneously, making it difficult to determine which reaction is causing which result. See Cleland et al, “The Development of Stable Protein Formulations: A Close Look at Protein Aggregation, Deamidation, and Oxidation”, Critical Reviews in Therapeutic Drug Carrier Systems, 10(4):307-377 (1993)).

### SUMMARY OF THE INVENTION

Specifically, the present invention relates to stable, lyophilized, high concentration immunoglobulin formulations. There are three steps in preparing the present formulations, including preparing an aqueous pre-lyophilized formulation, a lyophilization step, and a reconstitution step.

This invention is directed to a stable lyophilized formulation prepared by lyophilizing an aqueous formulation, wherein the aqueous formulation comprises about 40 mg/ml to about 50 mg/ml of an immunoglobulin in a buffer, polysorbate, and sucrose. In a preferred embodiment of the invention, the aqueous pre-lyophilized formulation comprises (a) about 30 mg/ml to about 60 mg/ml natalizumab; (b) a buffer having a pH of about 5.5 to about 6.5; (c) about 20 mg/ml to about 50 mg/ml sucrose; and (d) about 0.02% to about 0.08% polysorbate. In a more preferred embodiment, the aqueous pre-lyophilized formulation comprises (a) about 40 mg/ml natalizumab; about 6 mM histidine, pH about 6.0; about 41 mg/ml sucrose; and (d) about 0.04% polysorbate 80.

The lyophilized formulation retains the stability of the immunoglobulin, and prevents the immunoglobulins intended for administration to human subjects from forming aggregates and/or particulates in the final product. This lyophilized formulation is stable at room temperature for at least three months, preferably 6 months, and more preferably one year. The lyophilized formulation is also stable at 2-8° C. for 1 year, preferably 2 years. This lyophilized formulation has a short reconstitution time of less than 10 minutes, and after reconstitution is suitable for parenteral administration such as intramuscular, subcutaneous, intravenous, or intraperitoneal injection.

The lyophilized formulation is reconstituted with a liquid to a clarified solution containing about 80-160 mg/ml immunoglobulin concentration. In a preferred embodiment, the reconstituted formulation comprises (i) about 80 mg/ml to about 160 mg/ml natalizumab; (ii) about 18 mM histidine at a pH of about 6.0; (iii) about 123 mg/ml sucrose; and (iv) about 0.12% polysorbate 80. In a more preferred embodiment, the reconstituted formulation comprises about 120 mg/ml natalizumab.

The pre-lyophilized formulation of the present invention can be lyophilized using appropriate drying parameters. The following drying parameters are preferred: a primary drying phase temperature of about −25° C. and pressure between about 80 mTorr to about 120 mTorr; and a secondary drying phase at about 20° C., and pressure between about 80 mTorr to 120 mTorr.

This invention further provides methods for making and for using the reconstituted formulations.

### BRIEF DESCRIPTION OF THE DRAWINGS

FIG. 1 shows the formation of high molecular weight species at 30°.

FIG. 2 shows the formation of high molecular weight species at 40° C.

FIG. 3 shows the formation of low molecular weight species at 30° C. in the prelyophilized solutions.

FIG. 4 shows the formation of low molecular weight species at 40° C. in the pre-lyophilized solutions.

FIG. 5 shows the total loss of monomer at 5° C. due to formation of both high and low molecular weight species.

FIG. 6 shows the total loss of monomer at 30° C. due to formation of both high and low molecular weight species.

FIG. 7 shows the total loss of monomer at 40° C. due to formation of both high and low molecular weight species.

FIG. 8 shows the loss of monomer in the lyophilized formulations with time at 5° C.

FIG. 9 shows the loss of monomer in the lyophilized formulations with time at 30° C.

FIG. 10 shows the loss of monomer in the lyophilized formulations with time at 40° C.

FIG. 11 shows the formation of low molecular weight species for each formulation at 30° C.

FIG. 12 shows the formation of low molecular weight species for each formulation at 40° C.

FIG. 13 shows the formation of high molecular weight species at 40° C. in pre-lyophilized formulations.

FIG. 14 shows the loss of monomer due to formation of aggregate at 5° C.

FIG. 15 shows the loss of monomer due to formation of aggregate at 30° C.

FIG. 16 shows the loss of monomer due to formation of aggregate at 40° C.

FIG. 17 shows the formation of high molecular weight species in pre-lyophilized samples at 40° C.

FIG. 18 shows the formation of low molecular weight species in pre-lyophilized samples at 40° C.

FIG. 19 shows the total loss of monomer at 40° C. due to formation of both high and low molecular weight species.

FIG. 20 shows reconstitution times at 40° C.

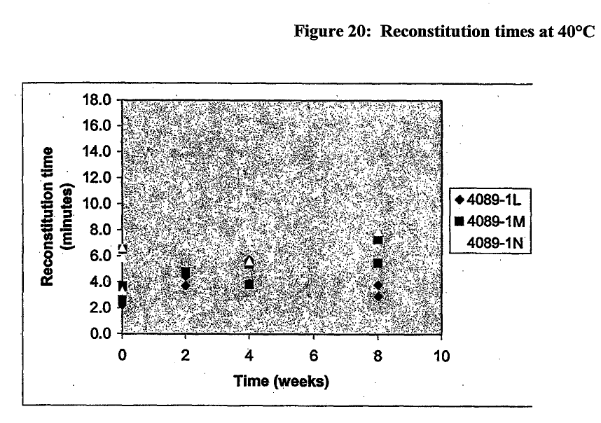

FIG. 21 shows the formation of high molecular weight species in lyophilized samples at 40° C.

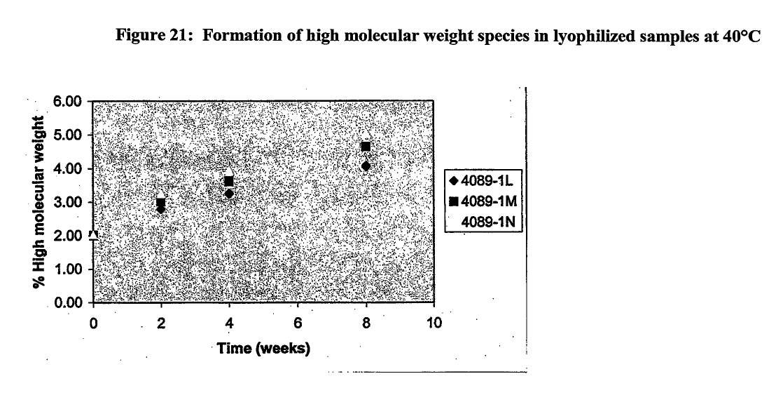

FIG. 22 shows the formation of low molecular weight species in lyophilized samples at 40° C.

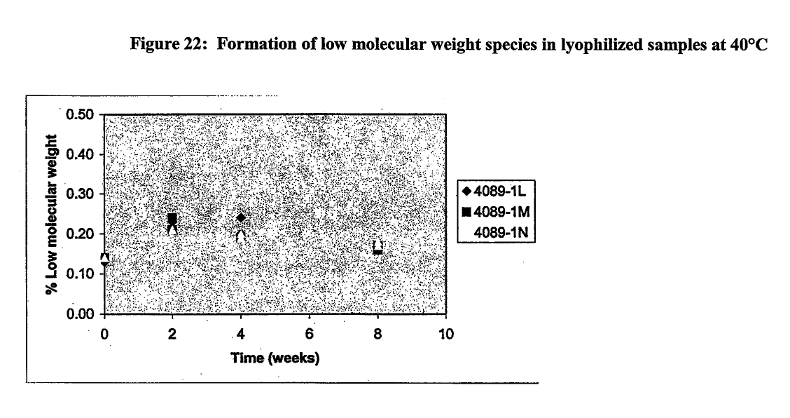

FIG. 23 shows the total loss of monomer at 40° C. due to formation of both high and low molecular weight species.

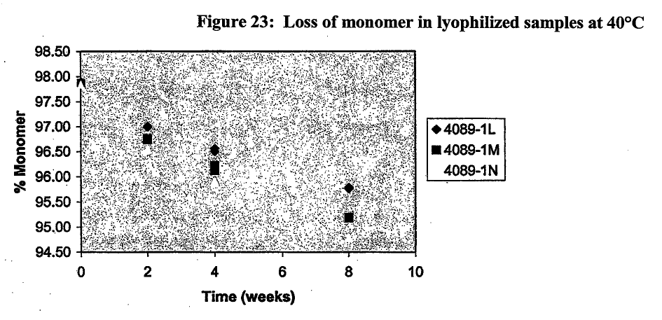

FIG. 24A shows the formation of high molecular weight species in pre-lyophilized samples at 5° C. FIG. 24 B shows the formation of low molecular weight species in pre-lyophilized samples at 5° C. FIG. 24 C shows loss of monomer in pre-lyophilized samples at 5° C.

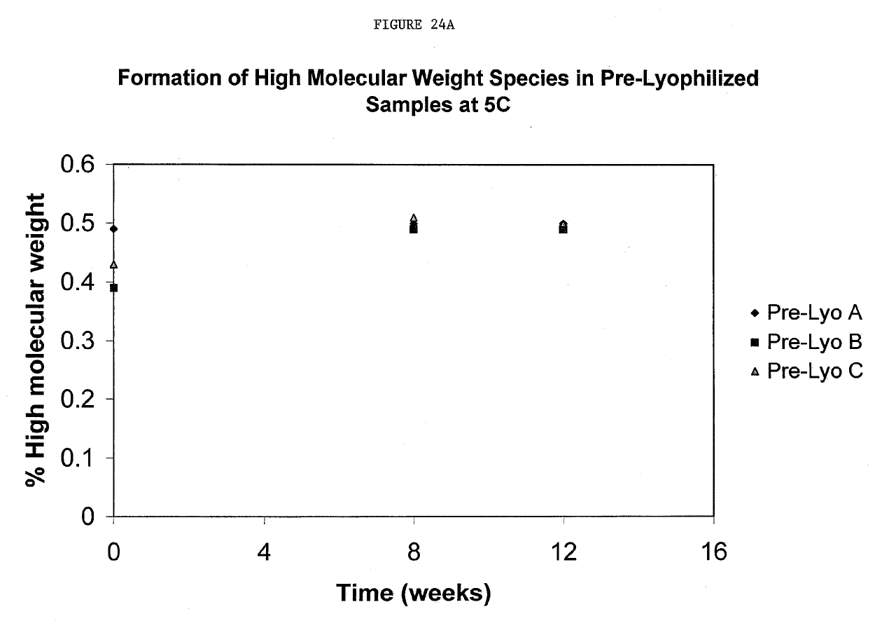

FIG. 25 shows formation of high molecular weight species in lyophilized samples at 40° C.

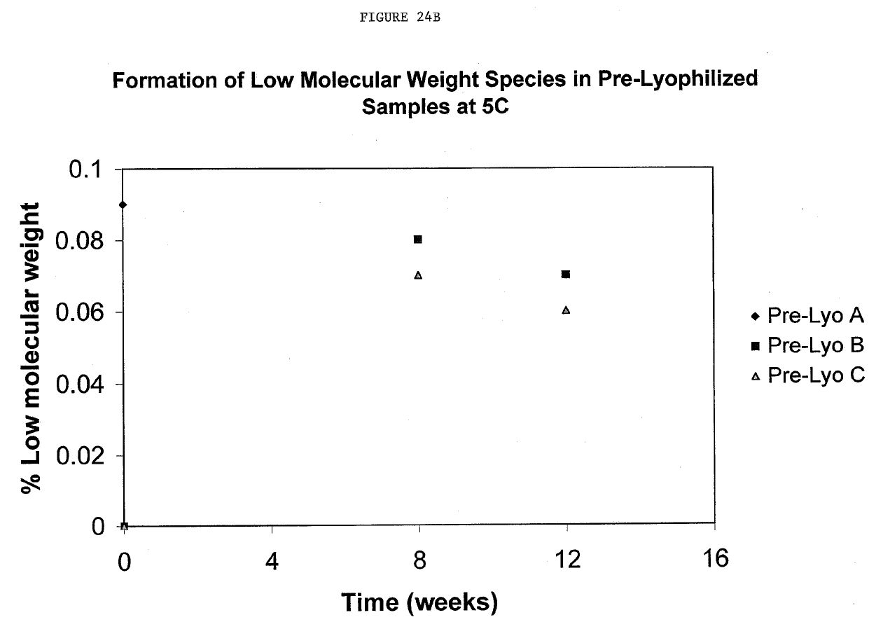

FIG. 26A shows formation of high molecular weight species at 40° C. in reconstituted samples.

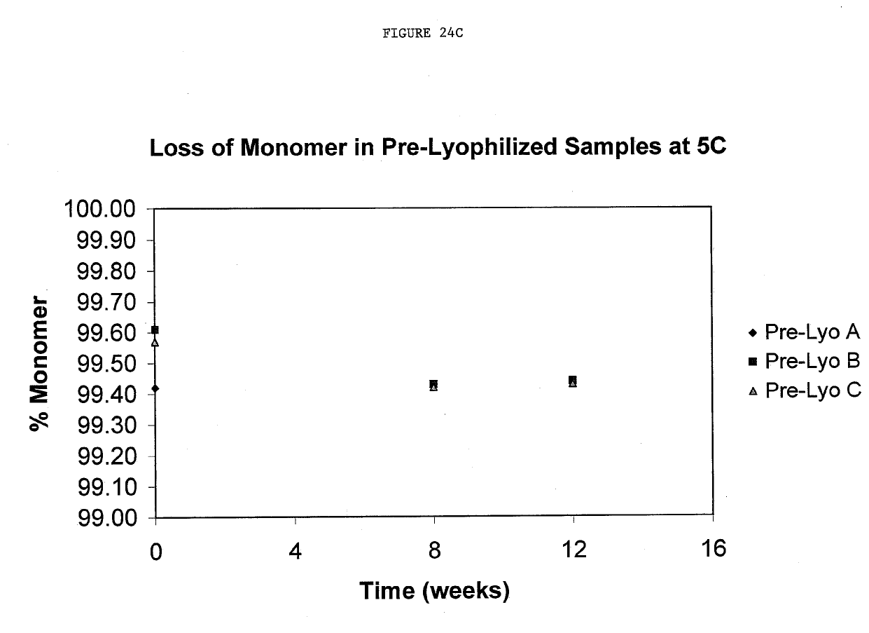

FIG. 26B shows formation of low molecular weight species at 40° C. in reconstituted samples.

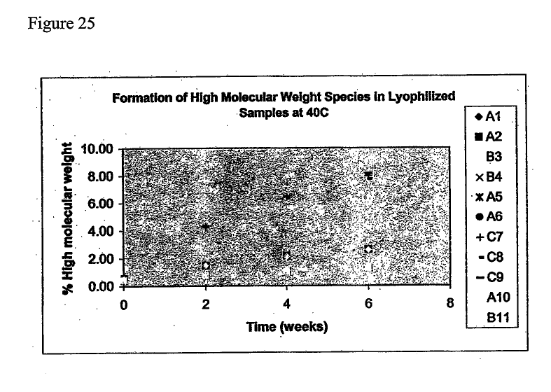

FIG. 26C shows loss of monomer at 40° C. in reconstituted samples. FIG. 26D shows reconstitution time.

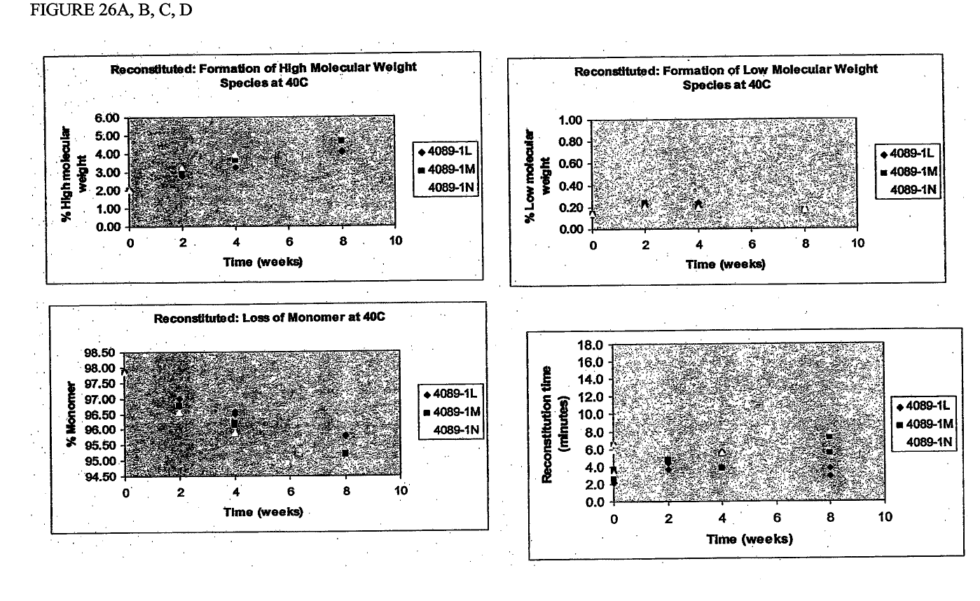

### DETAILED DESCRIPTION OF EMBODIMENTS OF THE INVENTION

### 1. Definitions

As used herein, the term “immunoglobulin” includes but is not limited to an antibody and antibody fragment (such as scFv, Fab, Fc, (Fab′)2), and other genetically engineered portions of antibodies. Depending on the amino acid sequence of the constant domain of their heavy chains, immunoglobulin can be assigned to different classes. There are five major classes of immunoglobulins: IgA, IgD, IgE, IgG and IgM. Several of these may be further divided into subclasses (isotypes), e.g., IgG1, IgG2, IgG3, and IgG4; IgA1 and IgA2.

The term “antibody” is used in the broadest sense and specifically covers monoclonal antibodies (including agonist and antagonist antibodies), antibody compositions with polyepitopic specificity, and antibody fragments (e.g., Fab, (Fab′)2, scFv and Fv), so long as they exhibit the desired biological activity. “Antibody” is meant to include polyclonal antibodies, monoclonal antibodies, humanized antibodies, human antibodies, primatized antibodies and other antibodies produced via genetic engineering.

The term “monoclonal antibody” as used herein refers to an antibody obtained from a population of substantially homogeneous antibodies, i.e., the individual antibodies comprising the population are identical except for possible naturally occurring modifications or glycosylation variants that may be present in minor amounts. The modifier “monoclonal” indicates the character of the antibody as being obtained from a substantially homogeneous population of antibodies, and is not to be construed as requiring production of the antibody by any particular method. The term “monoclonal antibodies” also includes “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. “Humanized” forms of non-human (e.g., murine, rabbit, bovine, equine, porcine, and the like) antibodies are chimeric immunoglobulins, immunoglobulin chains or fragments thereof (such as Fv, Fab, Fab′, F(ab′)2 or other antigen-binding subsequences of antibodies), which contain minimal sequence derived from non-human immunoglobulin.

The term “natalizumab” refers to the antibody also known as AN100226 (antibody code number), and is the active ingredient in TYSABRI® (trade name; formerly ANTEGREN®). The term “natalizumab” is the United States Adopted Name (USAN) (the official non-proprietary or generic name given to a pharmaceutical substance). Natalizumab is a recombinant, humanized anti-alpha-4 integrin antibody. Natalizumab is an IgG4 antibody. U.S. Pat. No. 5,840,299, incorporated herein by reference, describes how to make a recombinant humanized anti-alpha-4 integrin antibody, including natalizumab, using routine synthetic and molecular biology methods.

The terms “lyophilization,” “lyophilized,” and “freeze-dried” refer to a process by which the material to be dried is first frozen and then the ice or frozen solvent is removed by sublimation in a vacuum environment. One or more excipients may be included in pre-lyophilized formulations to enhance stability of the lyophilized product upon storage.

The term “pharmaceutical formulation” refers to preparations which are in such form as to permit the active ingredients to be effective, and which contains no additional components which are toxic to the subjects to which the formulation would be administered.

“Pharmaceutically acceptable” excipients (vehicles, additives) are those which can reasonably be administered to a subject mammal to provide an effective dose of the active ingredient employed.

“Reconstitution time” is the time that is required to rehydrate a lyophilized formulation with a solution to a particle-free clarified solution.

A “stable” formulation is one in which the protein therein essentially retains its physical stability and/or chemical stability and/or biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993). Stability can be measured at a selected temperature for a selected time period.

A “stable” lyophilized immunoglobulin formulation is a lyophilized antibody formulation with no significant changes observed at a refrigerated temperature (2-8° C.) for at least 12 months, preferably 2 years, and more preferably 3 years; or at room temperature (23-27° C.) for at least 3 months, preferably 6 months, and more preferably 1 year. The criteria for stability are as follows. No more than 10% of antibody monomer is degraded as measured by SEC-HPLC. Preferably no more than 5%, of antibody monomer is degraded as measured by SEC-HPLC. The rehydrated solution is colorless, or clear to slightly opalescent by visual analysis. The concentration, pH and osmolality of the formulation have no more than +/−10% change. Potency is within 70-130%, and preferably within 80-120%, of the control. No more than 10% of clipping is observed. Preferably, no more than 5% of clipping is observed. No more than 10% of aggregation is formed. Preferably, no more than 5% of aggregation is formed.

An immunoglobulin “retains its physical stability” in a pharmaceutical formulation if it shows no significant increase of aggregation, precipitation and/or denaturation upon visual examination of color and/or clarity, or as measured by LV light scattering, size exclusion chromatography (SEC) and dynamic light scattering. The changes of protein conformation can be evaluated by fluorescence spectroscopy, which determines the protein tertiary structure, and by FTIR spectroscopy, which determines the protein secondary structure.

An immunoglobulin “retains its chemical stability” in a pharmaceutical formulation, if it shows no significant chemical alteration. Chemical stability can be assessed by detecting and quantifying chemically altered forms of the protein. Degradation processes that often alter the protein chemical structure include hydrolysis or clipping (evaluated by methods such as size exclusion chromatography and SDS-PAGE), oxidation (evaluated by methods such as by peptide mapping in conjunction with mass spectroscopy or MALDI/TOF/MS), deamidation (evaluated by methods such as ion-exchange chromatography, capillary isoelectric focusing, peptide mapping, isoaspartic acid measurement), and isomerization (evaluated by measuring the isoaspartic acid content, peptide mapping, etc.).

An immunoglobulin “retains its biological activity” in a pharmaceutical formulation, if the biological activity of the immunoglobulin at a given time is within a predetermined range of the biological activity exhibited at the time the pharmaceutical formulation was prepared. The biological activity of an immunoglobulin can be determined, for example, by an antigen binding assay.

The term “isotonic” means that the formulation of interest has essentially the same osmotic pressure as human blood. Isotonic formulations will generally have an osmotic pressure from about 270-328 mOsm. Slightly hypotonic pressure is 250-269 and slightly hypertonic pressure is 328-350 mOsm. Osmotic pressure can be measured, for example, using a vapor pressure or ice-freezing type osmometer.

The term “buffer” encompasses those agents which maintain the solution pH in an acceptable range prior to lyophilization and may include histidine, succinate (sodium or potassium), phosphate (sodium or potassium), Tris (tris (hydroxymethyl) aminomethane), diethanolamine, citrate (sodium), gluconate, and other organic acid buffers.

“Tonicity Modifiers” include salts such as NaCl, KCl, MgCl2 CaCl2, etc. that can be used to control osmotic pressure. In addition, cryprotectants, lyoprotectants and/or bulking agents such as sucrose, mannitol, glycine etc. can serve as tonicity modifiers.

In the context of the present invention, a “therapeutically effective amount” of an immunoglobulin refers to an amount effective in the prevention or treatment of a disorder for the treatment of which the immunoglobulin is effective. A “disorder” is any condition that would benefit from treatment with the immunoglobulin. This includes chronic and acute disorders or diseases including those pathological conditions which predispose the mammal to the disorder in question. Preferably, the disorder is one that that can be treated and/or prevented by an immunoglobulin that recognizes and binds to alpha-4 integrin, such as natalizumab.

“Treatment” refers to both therapeutic treatment and prophylactic or preventative measures. Those in need of treatment include those already with the disorder as well as those in which the disorder is to be prevented.

A “preservative” is a compound which can be included in the formulation to essentially reduce bacterial action therein, thus facilitating the production of a multi-use formulation, for example. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol.

The term “patient” or “subject” is meant to include any mammal. A “mammal,” for purposes of treatment, refers to any animal classified as a mammal, including but not limited to humans, domestic and farm animals, and zoo, sports, or pet animals, such as dogs, horses, cats, cows, and the like. Preferably, the mammal is human. Preferably the disease or condition being treated in the mammal is one which is modulated when a therapeutically effective dose of natalizumab is administered.

### 2. Immunoglobulin Formulations

The compositions of this invention minimize the formation of aggregates and particulates and ensure that the immunoglobulin in solution maintains its immunoreactivity over time. The compositions comprise a sterile, pharmaceutically acceptable lyophilized formulation prepared from an aqueous pre-lyophilized formulation comprising an immunoglobulin in a buffer having a neutral or acidic pH (about 5.5 to about 6.5), sucrose, and a polysorbate.

In a preferred embodiment, the immunoglobulin is present in the pre-lyophilized formulation at a concentration of about 30 to about 60 mg/ml, more preferably about 40 to about 50 mg/ml, and even more preferably about 40 mg/ml. A preferred immunoglobulin is an IgG antibody, more preferably an IgG4 antibody, even more preferably a humanized recombinant IgG4 antibody, and most preferably natalizumab.

A buffer of pH 5.5 about to about 6.5 is used in the pre-lyophilized formulation. Preferably, the pH is about 6.0. Examples of suitable buffers include histidine, succinate (such as sodium succinate), gluconate, citrate and other organic acid buffers. A preferred pre-lyophilized formulation contains histidine, preferably about 1 to about 12 mM histidine. A even more preferred pre-lyophilized formulation contains about 6 mM histidine.

The pre-lyophilized formulation also comprises sucrose. Suitable concentrations of sucrose are in the range of about 20 to about 50 mg/ml, preferably about 41 mg/ml.

The pre-lyophilized formulation also comprises a polysorbate, such as polysorbate 20 or polysorbate 80 (i.e. Tween 20 and Tween 80, respectively) and poloxamer (e.g. poloxamer 188). In a preferred embodiment, the polysorbate is polysorbate 80. The polysorbate is preferably present at a weight per volume concentration of about 0.02 to about 0.08%, more preferably about 0.04%.

The weight ratio of immunoglobulin to sucrose in the pre-lyophilized formulation is preferably in the range of about 2:1 to about 0.5:1, more preferably about 1:1. The molar ratio of immunoglobulin to sucrose is about 300:1 to about 500:1, preferably about 400:1 to 500:1, more preferably about 450:1.

A bulking agent that provides good lyophilized cake properties, such as serine, glycine, and mannitol, can be optionally added to the present composition. These agents also contribute to the tonicity of the formulations and may provide protection to the freeze-thaw process and improve long-term stability. In addition, tonicity modifiers can be added to the formulation to control osmotic pressure. The formulation may further comprise one or more preservatives.

A preferred pre-lyophilized formulation is a formulation comprising about 40 mg/ml natalizumab, about 6 mM histidine (pH about 6), about 0.04% polysorbate 80, and about 41 mg/ml sucrose. The above pre-lyophilized formulation is lyophilized to form a dry, stable powder, which can be easily reconstituted to a particle-free solution suitable for administering to humans.

Lyophilization is a freeze drying process that is often used in the preparation of pharmaceutical products to preserve their biological activity. The liquid composition is prepared, then lyophilized to form a dry cake-like product. The process generally involves drying a previously frozen sample in a vacuum to remove the ice, leaving the non-water components intact, in the form of a powdery or cake-like substance. The lyophilized product can be stored for prolonged periods of time, and at elevated temperatures, without loss of biological activity, and can be readily reconstituted into a particle-free solution by the addition of an appropriate diluent. An appropriate diluent can be any liquid which is biologically acceptable and in which the lyophilized powder is completely soluble. Water, particularly sterile, pyrogen-free water, is a preferred diluent, since it does not include salts or other compounds which may affect the stability of the antibody. The advantage of lyophilization is that the water content is reduced to a level that greatly reduce the various molecular events which lead to instability of the product upon long-term storage. The lyophilized product is also more readily able to withstand the physical stresses of shipping. The reconstituted product is particle free, thus it can be administered without prior filtration.

The pre-lyophilized formulation of the present invention can be lyophilized using appropriate freezing and drying parameters. For example, parameters may include a pre-freeze to holding at about 10° C. to about −10° C. for about 10-30 minutes. Freezing parameters may include freezing for −50° C. to −70° C. over a period of about 45 minutes to about 75 minutes. Parameters for the additional freeze step may include freezing at −40° C. to about −60° C. Drying parameters may include a primary drying phase temperature of about −10° C. to −30 C and pressure between about 40 mTorr to about 120 mTorr; and a secondary drying phase at about 10° C. to about 25° C., using pressure between about 40 mTorr to 120 mTorr. A preferred total cycle time is about 60 to 100 hours. A preferred Lyophilization cycle may include a pre-freeze step, a freeze step, a primary drying step, and secondary drying step. Considerations for a lyophilization cycle include freeze temperature, pressure, primary drying, secondary drying, and cycle time.

For example, preferred lyophilization cycle parameters may be as follows:

First, pre-freeze, holding at 0° C. for 15 minutes.

For freezing, ramp to −60° C. over 60 minutes. Hold at −60° C. for 60 minutes.

In an additional freeze step, ramp to −50° C. and hold for 30 minutes.

For primary drying, ramp to −15° C. over 45 minutes with a pressure drop to 50 mTorr. Hold for 54 hours at −15° C. and 50 mT pressure.

For secondary drying, ramp to 20° C. over 35 minutes and hold for 24 hours.

The total cycle time is 82 hours.

This lyophilized product retains the stability of immunological activity of the immunoglobulin, and prevents the immunoglobulins intended for administration to human subjects from physical and chemical degradation in the final product.

The lyophilized product is rehydrated at the time of use in a diluent (e.g., sterile water or saline) to yield a particle-free solution. The reconstituted antibody solution is particle-free even after prolonged storage of the lyophilized cake at ambient temperature. The reconstituted solution can be administered parenterally, preferably intramuscularly or subcutaneously, to the subject.

An important characteristic of the lyophilized product is the reconstitution time or the time taken to rehydrate the product. To enable very fast and complete rehydration, it is important to have a cake with a highly porous structure. The cake structure is a function of a number of parameters including the protein concentration, excipient type and concentration, and the process parameters of the lyophilization cycle. Generally the reconstitution time increases as the protein concentration increases, and thus, a short reconstitution time is an important goal in the development of high concentration lyophilized antibody formulations. A long reconstitution time can deteriorate the product quality due to the longer exposure of the protein to a more concentrated solution. In addition, at the user end, the product cannot be administered until the product is completely rehydrated. This is to ensure that the product is particulate-free, the correct dosage is administered, and its sterility is unaffected. Thus, quick rehydration, such as a rehydration time of less than ten minutes, offers more convenience to the patients and the physicians.

In lyophilized products, the desired dosage can be obtained by lyophilizing the formulation at the target protein concentration and reconstituting the product with the same volume as that of the starting fill volume. The desired dosage can also be obtained by lyophilizing a larger volume of a diluted formulation, and reconstituting it with a less volume. For example, if a desired product dosage is 100 mg of protein in 1 mL of the formulation, the formulations can be lyophilized with the following liquid configurations: 1 mL of 100 mg/mL, 2 mL of 50 mg/ml, or 4 mL of 25 mg/mL protein formulation. In all cases, the final product can be reconstituted with 1 mL diluent to obtain the target protein concentration of 100 mg/mL. However, as the protein concentration in the pre-lyophilized formulation is reduced, the fill volume increases proportionately. This correspondingly increases the length of the lyophilization cycle (especially the primary drying time), and thus significantly adds to the cost of the product. For example, if 1 mL fill volume (1 mm height in vial) of frozen material takes approximately 1 hour to sublimate its free water, then 10 mL fill volume (10 mm height) of frozen product will take approximately 10 hours of primary drying time. Therefore, it is advantageous to have a concentrated pre-lyophilized formulation (with immunoglobulin concentration about 40 mg/ml to about 50 mg/ml) such that the lyophilization process will be more efficient.

The present invention provides a highly concentrated pre-lyophilized immunoglobulin formulation (about 40 mg/ml to about 50 mg/ml), which is lyophilized efficiently and effectively to a dry formulation that retains the biological, physical and chemical stability of the immunoglobulin. The dry formulation is stable for storage at least for 3 months, preferably 6 months, at room temperature. The dry formulation can be reconstituted within a short time of less than ten minutes to a particle-free solution containing about 80 mg/ml to about 160 mg/ml immunoglobulin. Such highly concentrated antibody solution is ready for parenteral administration such as intravenous, intramuscular, intraperitoneal, or subcutaneous injection.

A preferred reconstituted product comprises about 80 mg/ml to about 160 mg/ml natalizumab, more preferably about 120 mg/ml natalizumab; about 123 mg/ml sucrose; about 0.12% polysorbate 80; and about 18 mM histidine at about pH 6.0.

### 3. Analytical Methods

Analytical methods for evaluating the product stability include size exclusion chromatography (SEC), dynamic light scattering test (DLS), differential scanning calorimetery (DSC), iso-asp quantification, potency, UV at 340 nm, UV spectroscopy, and FTIR. SEC (J. Pharm. Scien., 83:1645-1650, (1994); Pharm. Res., 11:485 (1994); J. Pharm. Bio. Anal., 15:1928 (1997); J. Pharm. Bio. Anal., 14:1133-1140 (1986)), which measures percent monomer in the product and gives information of the amount of soluble aggregates. The potency or bioidentity of an antibody can be measured by its ability to bind to its antigen. The specific binding of an antibody to its antigen can be quantitated by any method known to those skilled in the art, for example, an immunoassay, such as ELISA (enzyme-linked immunosorbant assay). There methods are merely exemplary of methods for evaluating product stability well known to one of skill in the art. By way of example, UV at 340 nm measures scattered light intensity at 340 nm and gives information about the amounts of soluble and insoluble aggregates. UV spectroscopy measures absorbance at 278 nm and gives information of protein concentration. FTIR (Eur. J. Pharm. Biopharm., 45:231 (1998); Pharm. Res., 12:1250 (1995); J. Pharm. Scien., 85:1290 (1996); J. Pharm. Scien., 87:1069 (1998)) measures IR spectrum in the amide one region, and gives information of protein secondary structure. Particular analytical methods are further set forth in the experimental section, supra.

### 4. Use of the Reconstituted Formulations

The reconstituted immunoglobulin formulations of the present invention may be administered to a mammal in need of treatment with the immunoglobulin, in accordance with known methods. These methods may include, but are not limited to intravenous administration as a bolus or by continuous infusion over a period of time, by intramuscular, intraperitoneal, intracerobrospinal, subcutaneous, intra-articular, intrasynovial, intrathecal, oral, topical, or inhalation routes. In preferred embodiments, the immunoglobulin formulation is administered to the mammal by intramuscular or subcutaneous administration. A typical daily dose may range from about 1 μg/kg to about 200 mg/kg subject weight or more, more preferably from about 0.01 mg/kg to about 150 mg/kg subject weight, more preferably, from about 0.1 mg/kg to about 100 mg/kg subject weight, more preferably about 1 mg/kg to about 75 mg/kg subject weight, and most preferably about 3 mg/kg to about 6 mg/kg subject weight. Typically, the physician will administer immunoglobulin until a dosage is reached that achieves the desired effect. The progress of this therapy may be easily monitored by conventional methods and assays.

The appropriate dosage of the immunoglobulin will depend, for example, on the condition to be treated, the severity and course of the condition, whether the immunoglobulin is administered for preventive or therapeutic purposes, previous therapy, the patient's clinical history and response to the immunoglobulin, the type of immunoglobulin used, and the discretion of the attending physician. Typically, the clinician will administer immunoglobulin until a dosage is reached that achieves the desired effect. The progress of this therapy can be easily monitored by conventional assays.

The immunoglobulin is suitably administered to the patient at one time or over a series of treatments and may be administered to the patient at any time from diagnosis onwards. The immunoglobulin may be administered as the sole treatment or in conjunction with other drugs or therapies useful in treating the condition in question. As used herein, two (or more) agents are said to be administered in combination when the two agents are administered simultaneously or are administered independently in a fashion such that the agents will act contemporaneously. For example, natalizumab formulations of this invention can be administered in combination with other therapeutic agents or physical therapies for the treatment of rheumatoid arthritis, multiple sclerosis (MS), Crohn's Disease, and other alpha-4 mediated diseases.

The invention is illustrated further by the following examples, which are not to be construed as limiting the invention in scope of the specific procedures described in them.

### EXAMPLES

### Example 1

A comparative study of high concentration reconstituted lyophilized and liquid formulations of natalizumab was performed in cynomolgus monkeys. The results of the study showed the reconstituted lyophilized formulation of natalizumab produced expected pharmacokinetic and pharmacodynamic profiles very similar to the liquid formulation.

High concentration liquid and reconstituted lyophilized formulations of natalizumab were evaluated to compare their respective pharmacokinetic/pharmacodynamic profiles, relative bioavailability, and local tolerability following subcutaneous (SC) and intramuscular (IM) dosing. Both the liquid (150 mg/mL) and reconstituted lyophilized (120 mg/mL) high concentration formulations were administered by each extravascular route on Day 1 and their pharmacokinetic/pharmacodynamic profiles were evaluated through Day 36. A single 30 mg dose of commercial liquid natalizumab was also administered on Day 1 to determine the relative bioavailability of the high concentration formulations. Animals in the SC and IM dose groups were administered a second injection on Day 36 and injection site biopsies were performed on Day 39 to assess local tolerability.

The test articles, commercial liquid natalizumab, liquid high concentration natalizumab and lyophilized high concentration natalizumab were supplied by Biogen Idec. The reconstituted lyophilized natalizumab reconstitution fluid, Sterile Water for Injection, was supplied as an aqueous solution. Fresh vials of the stock test articles were used on each dosing day and were formulated to yield dosing solutions at the appropriate concentrations.

Thirty experimentally naive cynomolgus monkeys (15 males and 15 females) were assigned to five dose groups as shown in the following table 1:

TABLE 1

| Group | Number | | Test Article | Dose Level | Dose Volume | Dose Solution |
|---|---|---|---|---|---|---|
| No. | of M/F | Route. | Formulation | (mg/animal) | (mL/animal) | Conc. (mg/ml) |
| 1 | 3/3 | IV | Liquid* | 30 | 1.5 | 20 |
| 2 | 3/3 | SC | Liquid** | 150 | 1.0 | 150 |
| 3 | 3/3 | IM | Liquid** | 150 | 1.0 | 150 |
| 4 | 3/3 | SC | Lyophilized*** | 120 | 1.0 | 120 |
| 6 | 3/3 | IM | Lyophilized*** | 120 | 1.0 | 120 |

*Commercial liquid natalizumab;
**Liquid high concentration natalizumab;
***Reconstituted lyophilized high concentration natalizumab
IV = intravenous;
SC = subcutaneous;
IM = intramuscular;
M/F = male/female

Pharmacologic-related increases in peripheral blood lymphocytes (and to a lesser extent, eosinophils, basophils, and unclassified cells) compared to prestudy baseline values were noted in all groups and considered an expected pharmacodynamic effect of the test article. Increased levels of affected white blood cells generally persisted longer in duration in individual animals receiving the higher SC and IM doses of natalizumab (e.g., from Day 8 through Day 36) versus the lower IV dose of the commercial formulation (typically Day 8 through Day 15). However, there were no consistent or apparent differences in magnitude or duration of response as a function of liquid versus lyophilized high concentration formulations of the test article, sex, or route of administration.

Mean Tₘₐₓ was achieved most rapidly for the commercial liquid IV dose group, and more rapidly for IM groups than the SC groups across high concentration formulations. Mean Cₘₐₓ values were less than dose proportional across routes when compared to the commercial liquid IV dose group and were consistent across high concentration formulations when administered extravascularly. The mean t½ values were consistent across all dose groups irrespective of route.

Both mean AUCₗₐₛₜ and AUC_{inf} were greater than dose proportional in the SC and IM dose groups irrespective of formulation indicating complete absorption (i.e. relative bioavailability of 100%), and were also quite consistent across formulations when administered extravascularly.

Expected test article-related increases in circulating lymphocyte counts, and to a lesser extent, eosinophil, basophil and unclassified cell counts were seen, as compared to prestudy baselines present in all dose groups. These results were consistent with a 4-integrin saturation profiles and were attributed to the pharmacologic effects of the natalizumab. The duration of the increases in affected white blood cell counts was dose-dependent and typically greater for the liquid high concentration natalizumab and lyophilized high concentration natalizumab-treated groups (e.g., from Day 8 up to Day 36) compared to the group receiving the commercial liquid formulation intravenously (typically Day 8 to Day 15). There were no consistent differences observed relative to the changes in these white blood cell populations between the high concentration liquid and reconstituted lyophilized formulations of natalizumab, or any difference that was considered related to the route of administration (subcutaneous or intramuscular injection) or the sex of the animals.

In summary, reconstituted lyophilized high concentration natalizumab formulations following subcutaneous and intramuscular injections in cynomolgus monkeys at doses of 120 mg (reconstituted lyophilized formulation) were well tolerated and produced expected pharmacologic-related increases in peripheral blood lymphocyte counts that were comparable to, but of slightly greater persistence than after a single 30 mg intravenous dose of the commercial liquid formulation.

### Example

Natalizumab is currently delivered as an IV infusion over a period of 1 to 2 hours. This requires the patient to visit a hospital or specialized infusion center. In order to make delivery of natalizumab more convenient, a subcutaneous administration is desired. It was advantageous to develop a lyophilized formulation from a bulk drug substance at a lower concentration that could then be reconstituted at a higher concentration. The present study described here was designed to screen the effect of various starting and final protein concentrations on stability using sucrose as an excipient (Study A). The second part of the study was designed to screen other well-known lyo-protectants and excipients for their effects on the protein stability (Study B). Efforts were also made to examine solutions that would be feasible with regards to manufacturability in terms of fill volumes and reconstitution volumes.

In Study A, four formulations were prepared to examine the effect of the starting and final protein concentration on real time and accelerated stability for both the pre-lyophilized bulk and the lyophilized cake. All four of the pre-lyophilized formulations exhibited sufficient stability to be used as pre-lyophilized bulks for hold times of up to 6 months at 5° C. Two lyophilized formulations exhibited sufficient formulation characteristics and stability to be considered as candidates for further formulation development. One of these formulations consisted of a starting concentration of 40 mg/mL and a reconstituted concentration of 100 mg/mL with a 1:1 weight ratio of protein to sucrose. The second candidate formulation contained a 50 mg/mL starting concentration and a reconstitution concentration of 200 mg/mL with a 2:1 weight ratio of protein to sucrose. Both formulations contained a histidine buffer and polysorbate 80.

In addition, a high concentration liquid formulation with the same excipients profile as the current natalizumab (Tysabri™) formulation was prepared. At 5° C. this formulation demonstrated sufficient stability to aggregate formation to be considered a candidate for further development, although the rate of deamidation was more rapid than in the lyophilized formulations. At accelerated temperatures this formulation showed a tendency to form low molecular weight degradation products, which were not observed to the same extent in the lyophilized formulations.

### Experimental Design

### Materials

Natalizumab used for these studies was prepared from natalizumab supplied by Biogenldec, manufactured on February 2003. This material had been formulated and vialed, therefore the material needed to be pooled and the polysorbate 80 removed prior to use in these formulation studies. Briefly, this was accomplished by diafiltration into a low ionic strength, high pH (10 mM tris, 10 mM NaCl, pH 8.5) buffer. The material was bound to a DEAE-Sepharose column under these conditions and then eluted using 10 mM sodium phosphate, 140 mM NaCl, at pH 6. The column eluate was then diafiltered into 6 mM histidine, pH 6 and concentrated to between 70 and 100 mg/mL prior to further formulation.

### Chemicals and Reagents

All chemicals and reagents used in this study, except as noted were purchased from VWR and were ACS grade or better. USP grade reagents were used as excipients when available. Polysorbate 80 was purchased from Sigma (Cat # P6474), and was vegetable derived and low peroxide.

### Formulations for Study A

The following formulations were prepared by diluting stock solutions of the excipients and active to the desired concentrations as indicated in Table 2: Study A formulation parameters. All formulations were prepared at pH 6.

TABLE 2
Study A formulation parameters.

| Formulation | | |
|---|---|---|
| ID | Pre-lyo Concentrations | Reconstituted Concentrations |
| 3944-18A | 20 mg/mL Ab, 20 mg/mL | 100 mg/mL Ab, 100 mg/mL sucrose, |
| | sucrose, 6 mM his, 0.02% PS80 | 30 mM his, 0.1% PS80 |
| 3944-18B | 40 mg/mL Ab, 40 mg/mL sucrose, 6 mM | 100 mg/mL Ab, 100 mg/mL sucrose, |
| | his, 0.02% PS80 | 15 mM his, 0.05% PS80 |
| 3944-18C | 50 mg/mL Ab, 25 mg/mL sucrose, 6 mM | 200 mg/mL Ab, 100 mg/mL sucrose, |
| | his, 0.02% PS80 | 24 mM his, 0.08% PS80 |
| 3944-18D | 75 mg/mL Ab, 40 mg/mL sucrose, 6 mM | 225 mg/mL Ab, 120 mg/mL sucrose, |
| | his, 0.02% PS80 | 18 mM his, 0.06% PS80 |
| 3944-18L | 100 mg/mL, 10 mM sodium | NA |
| | phosphate, 140 mM NaCl, pH | |
| | 6, .02% PS80 | |

Abbreviations:
Ab = antibody,
his = histidine,
PS80 = polysorbate 80

### Formulations for Study B

The following formulations were prepared by diluting stock solutions of the excipients and active to the desired concentrations as shown in Table 3: Study B formulation parameters. All formulations were at pH 6.

TABLE 3
Study B formulation parameters.

| Formulation | | |
|---|---|---|
| ID | Pre-lyo Concentrations | Reconstituted Concentrations |
| 3976-4C | 50 mg/mL Ab, 25 mg/mL sucrose, 6 mM | 200 mg/mL Ab, 100 mg/mL sucrose, 24 mM |
| | his, 0.02% PS80 | his, 0.08% PS80 |
| 3976-4G | 50 mg/mL Ab, 25 mg/mL sucrose, 15 mM | 200 mg/mL Ab, 100 mg/mL sucrose, 60 mM |
| | NaCl, 6 mM his, 0.02% PS80 | NaCl, 24 mM |
| | | his, 0.08% PS80 |
| 3976-4H | 50 mg/mL Ab, 25 mg/mL sucrose, 6 mM | 200 mg/mL Ab, 100 mg/mL sucrose, 24 mM |
| | his, 0.02% PS20 | his, 0.08% PS20 |
| 3976-4I | 50 mg/mL Ab, 25 mg/mL trehalose, 6 mM | 200 mg/mL Ab, 100 mg/mL trehalose, |
| | his, 0.02% PS80 | 24 mM his, 0.08% PS80 |
| 3976-4K | 40 mg/mL Ab, 25 mg/mL sucrose, 6 mM | 160 mg/mL Ab, 100 mg/mL sucrose, 24 mM |
| | his, 0.02% PS80 | his, 0.08% PS80 |

Abbreviations:
Ab = antibody,
his = histidine,
PS80 = polysorbate 80

### Methods

### Preparation of Formulations

The solutions were sterile filtered and filled as indicated into sterile glass vials. All samples for pre-lyophilized analysis and 3944-18L were filled at 0.5 mL into 2 cc vials, stoppered and capped. Formulas to be lyophilized were filled at the following volumes into 5 cc Kimble vials: Formulation 3944-18A —2.5 mL, Formulation 3944-18B —1.25 mL, Formulation 3944-18C —2 mL, Formulation 3944-18D —1.5 mL. All formulations for Study B were filled at 2 mL in 5 cc Kimble vials.

### Lyophilization Cycles

For Study A, the vials were frozen at −20° C. The lyophilizer malfunctioned and the temperature was dropped to −70° C. for the weekend. Subsequent attempts to restart the lyophilization cycle resulted in the temperature rising to 3-4° C. prior to re-freezing. The lyophilizer was restarted and the cycle continued. Primary drying was performed at −20° C. for 20 hours with 100 mTorr vacuum. The temperature was then ramped to 20° C. over 3 hours and held for 30 hours at 100 mTorr for secondary drying.

For Study B, the vials were frozen at −50° C. for 2 hours to ensure uniform freezing. The temperature was then taken to −40° C. for 20 min under vacuum of 100 mTorr. Then the temperature was ramped to −25° C. over 20 minutes with 100 mTorr vacuum and primary drying proceeded for 20 hours. The shelf temperature was ramped slowly to 20° C. over 10 hours to begin secondary drying at 100 mTorr, and then held at 20° C. for 4 hours.

### Study Set-Up

Vials of pre-lyophilized liquid, the high concentration liquid control, and lyophilized cakes were placed at 5, 30 and 40° C. Samples stored at 40° C. were assayed at 2, 4, 8 and 12 weeks. Samples stored at 30° C. were assayed at 3, 6, 9, and 12 weeks. Samples stored at 5° C. were assayed at 4, 8, and 12 weeks. Additional vials at 30° and 5° C. were analyzed at 6 months and 1 year for some formulations. All formulations were assayed pre- and post-lyophilization at time zero.

### Assays

The samples were assayed by the following methods. Not all assays were performed at all time points. With the exception of the zero time point for pre-lyophilized samples, duplicate vials were sampled for each assay.

### Appearance by Visual Examination

All samples were examined visually and their appearance recorded. Lyophilized cakes were examined for color, uniformity, robustness and evidence of meltback. Liquid and reconstituted samples were examined for color, clarity and presence of particulates.

### Residual Moisture and Reconstitution Time

Lyophilized cakes were assayed for residual moisture using Karl Fischer at the zero time point (BOP 000-01290). Reconstitution time was measured by adding the appropriate volume of DI water, followed by gentle swirling. The time for the cake to completely dissolve was recorded (EOP 000-01292).

### Concentration

The concentration of all samples was measured. Samples were diluted to 1 mg/mL using natalizumab placebo. The UV absorbance was scanned from 400 to 240 nm using a Varian 300Bio Spectrophotometer and 1 cm pathlength cuvette at 200 nm/minute. The absorbance at lambda max was recorded and the concentration was determined by dividing that value by 1.498 (the absorptivity coefficient for natalizumab) and adjusting by the appropriate dilution.

### Turbidity

The absorption from 300 to 400 nm of a neat sample was measured using a 10 mm small volume cuvette (Starna Cells Inc. Cat #16.160-Q-10\Z20). The value recorded at 360 nm was reported.

### Size Exclusion Chromatography

The amount of monomer, high molecular weight species, dimer and low molecular weight species were determined using a modification of Biogen SOP 22d.505. The samples were loaded to the column neat and the load volume was adjusted to allow a mass of approximately 400 μg on the column. The mass of the reference material load was also adjusted to be comparable. Detection was recorded at both 215 and 280 nm, however the main peak was off scale at 215 nm, so the 280 nm trace was used for calculations reported here.

### Cation Exchange Chromatography

Cation exchange chromatography was performed. Due to differences in the chromatography system, the gradient was modified slightly to allow the main peak to elute between 9 and 12 minutes. Since the system used was a binary pump the high salt wash was only performed after each set of analyses. There was no evidence in the high salt wash of any peaks eluting with late retention times. Since the high salt wash was not performed after each sample, the re-equilibration time was shortened to 7 minutes.

### Potency

The potency of selected samples was analyzed by the VCAM lysate assay (AAM 001-00965) and by the Jurkat cell assay (AAM 001-00700).

### Results of Study A

This study was designed as a screening study to examine the effect of both the initial protein concentration and the final protein concentration on lyophilized cake parameters, reconstitution time and stability. Preliminary studies had shown that sucrose provided sufficient short-term lyo-protectant properties to natalizumab, while mannitol did not. For this reason, initial screening was performed using sucrose as a lyo-protectant. Early studies had also shown that the optimal pH for natalizumab, was pH 6. Phosphate, citrate, and histidine buffers are most commonly used for buffering at this pH. Phosphate buffer is not optimal for use in lyophilization of proteins due to its pH shift upon freezing. Citrate buffer has been implicated in pain on injection in some subcutaneous formulations. Consequently, histidine was chosen as the preferred buffer species. The initial concentration was fixed at 6 mM for ease of preparation of formulations and to maintain the final concentration at or below 30 mM after reconstitution. For Study 3A, the polysorbate 80 concentration was maintained at 0.02% in the pre-lyophilized solutions as this had been shown to give sufficient protection to the protein and also to maintain final concentrations at or below 0.1%. The sucrose concentration was selected to maintain between a 1:1 and 2:1 weight ratio of protein to sugar while still maintaining a final solution that would be close to isotonic.

### Pre-Lyophilized and Liquid Formulations

The tables in the appendix contain all results recorded for all formulations. On visual examination, all formulations appeared colorless and clear to opalescent, without any visual particulates. As expected the opalescence increased somewhat with increasing protein concentration. None of the samples showed a change in protein concentration within the variation of the analysis and no trend was observed. The turbidity, as measured by absorbance at 360 nm, showed no change at 5° C. over 6 months or at 30° C. over 12 weeks of the study. The 40° C. pre-lyophilized samples showed a slight increase of turbidity with time for the samples at 40, 50 and 75 mg/mL. The 20 mg/mL pre-lyophilized sample and high concentration liquid did not demonstrate this trend in the 40° C. samples.

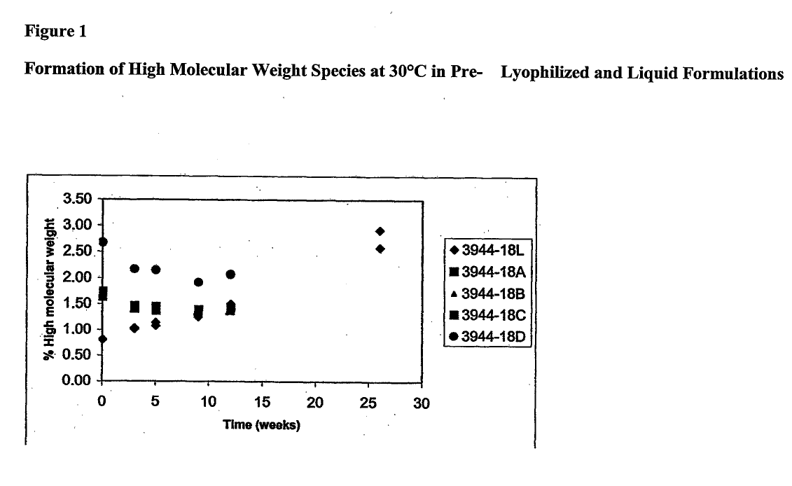

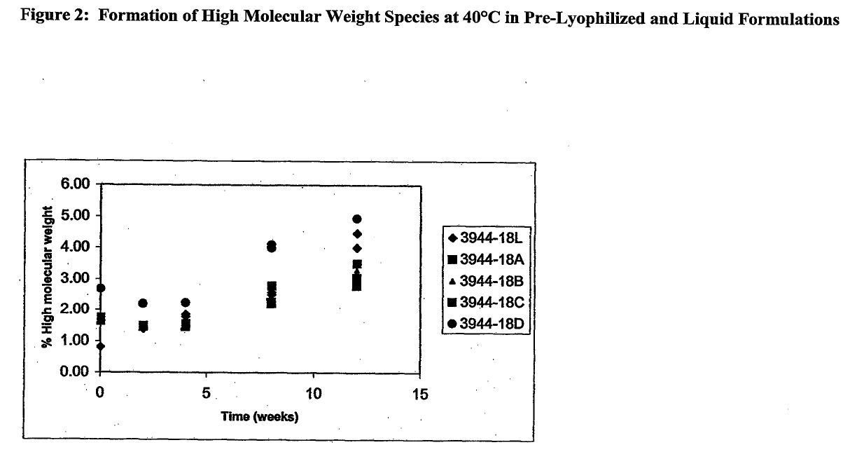

The formation of dimer, high molecular weight aggregate and low molecular weight species was monitored for all formulations. The tabular results are shown in the tables in the appendix. FIGS. 1 and 2 show the formation of high molecular weight species at 30° and 40° C. At 5° C. there was no change in the level of high molecular weight species in any formulation for the 6 months of storage.

The 5° C. data is shown herein. The results at 30° C. (FIG. 1) showed no trend in formation of high molecular weight species for any of the pre-lyophilized formulations after 12 weeks of storage. The high concentration liquid formulation showed a definite increase in high molecular weight species over the 6 month storage period at 30° C.

At 40° C., the formation of high molecular weight species did not appear to begin until after 4 weeks of storage in the pre-lyophilized formulations. Then the amount of high molecular weight species continues to increase. The high concentration liquid formulation at high ionic strength shows lower starting percentage of high molecular weight species, but the amount appears to increase at a constant rate over the 3 month study at 40° C.

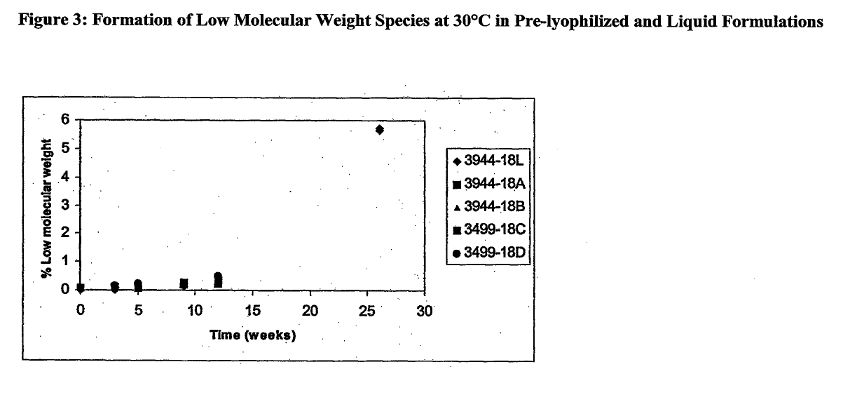

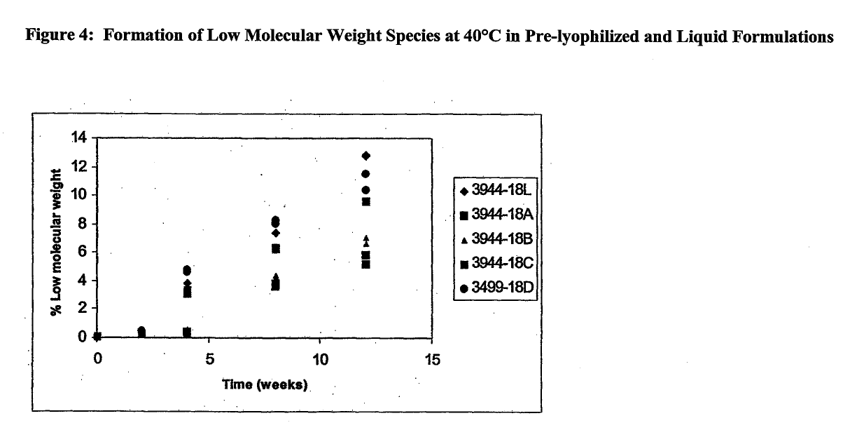

FIGS. 3 and 4 show the formation of low molecular weight species at 30 and 40° C. in the pre-lyophilized solutions. There was only a very small change in the level of low molecular weight species at 30° C. during 3 months of storage, but a definite upward trend was seen. Only the high concentration liquid was assayed at 6 months. At that time there was a considerable amount of low molecular weight species present, almost 6%.

There was a definite trend of formation of low molecular weight species during storage at 40° C. The rate of formation at this temperature appears to be somewhat concentration dependent as the higher concentration samples show a more rapid formation. Also, the formation of low molecular weight species at 30° and 40° C. appears to precede at a faster rate than the formation of high molecular weight species making this an important degradation pathway in the liquid samples.

There was very little change in the percentage of low molecular weight species over a 6 month time period for the samples stored at 5° C. This data is shown in the appendices, but not graphed here. All of the pre-lyophilized formulations went from an initial level of approximately 0.6-0.9% to 0.15% at 6 months. The high concentration liquid did not show any low molecular species even after 6 months of storage at 5° C.

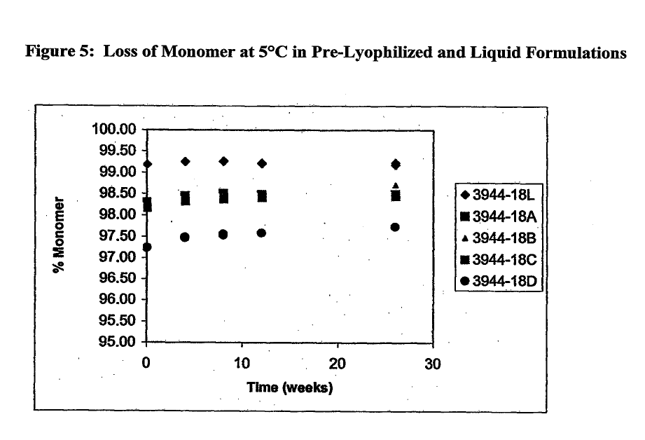

FIGS. 5, 6 and 7 show the total loss of monomer at each temperature due to formation of both high and low molecular weight species. At 2-8° C. there was essentially no change in the percent monomer. This would indicate that any of these formulations would be suitably stable as a pre-lyophilized formulation for storage up to 6 months at 2-8° C. The high concentration liquid formulation also shows no change at 2-8° C. The 30° C. samples show a very slight decrease in monomer. This change is due to formation of the low molecular weight species. This also indicates that any of these formulations would exhibit sufficient stability at ambient temperatures to allow for processing. The high concentration liquid shows a loss of monomer that is concurrent with the increase in low molecular weight species at 6 months. All formulations showed loss of monomer at 40° C. over the three-month period tested. The rate of loss appears to correlate roughly with the protein concentration with the liquid at 100 mg/mL and the 75 mg/mL pre-lyophilized formulations showing the most rapid rate of loss. Further investigation into the mechanism of degradation due to formation of the low molecular weight species could provide better stabilization to this route of degradation.

Selected samples were analyzed by cation exchange chromatography. In all cases the results showed a shift towards a more acidic species (deamidation) with storage based on time and temperature. There was no difference among the various pre-lyophilized formulations. Since deamidation is generally influenced by pH, ionic strength and certain buffer species, this result would be expected. The degradation of the high concentration liquid formulation was not significantly different from the pre-lyophilized formulations at 5° C. However, it showed significantly faster degradation at both 30 and 40° C. This is most likely due to the high ionic strength and the presence of phosphate buffer.

### Lyophilized Formulations

Tables 5-12 show the results for the stability of the samples stored as lyophilized formulations at 5, 30 and 40° C. All formulations were stored at 40° C. for 12 weeks, 30 and 5° C. for 6 months. In addition, Formulations 3944-18B and 3944-18C were analyzed after 1 year at 30 and 5° C.

The formulations were analyzed at the initial time point for residual moisture. The residual moisture was higher than desired, probably due to the problems experienced during the lyophilization cycle. The residual moisture was in the 5-6% range for these samples.

The time for reconstitution was measured and was directly correlated with the starting and final protein concentration. The cakes that were lyophilized from 75 mg/mL protein solutions took an average of 15-20 minutes to reconstitute. Samples lyophilized from 50 mg/mL took an average of 6-7 minutes, from 40 mg/mL took 5-6 minutes and from 20 mg/mL took 4-5 minutes. The values were variable but did not show any trend with regard to storage time or temperature.

The reconstituted vials were examined for appearance. All samples were clear to opalescent, colorless and free of particles. Two formulations that were stored at 30° C. for 1 year showed some slight yellow color. Turbidity was measured as a function of absorbance at 360 nm. All formulations showed a rise in turbidity when stored at 40° C. over a 12-week period. All formulations showed a less significant rise in turbidity when stored at 30° C. for up to 12 months. All formulations showed a slight rise in turbidity when stored at 5° C. for up to a year, except Formulation 3944-18C. (See tables in Appendix E-H). No change in protein concentration was observed outside the normal variation inherent in hand filling and reconstitution of the material.

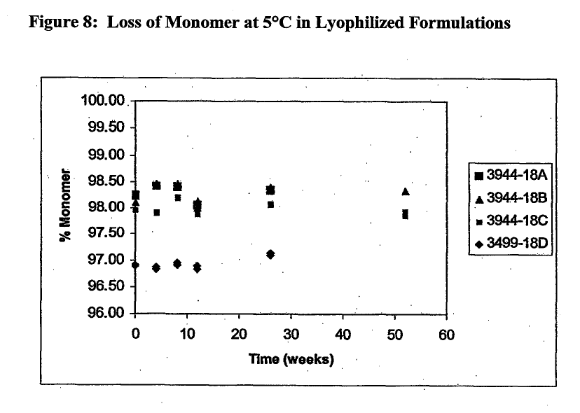

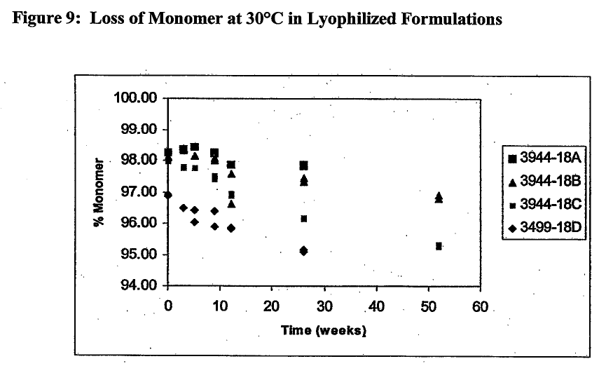

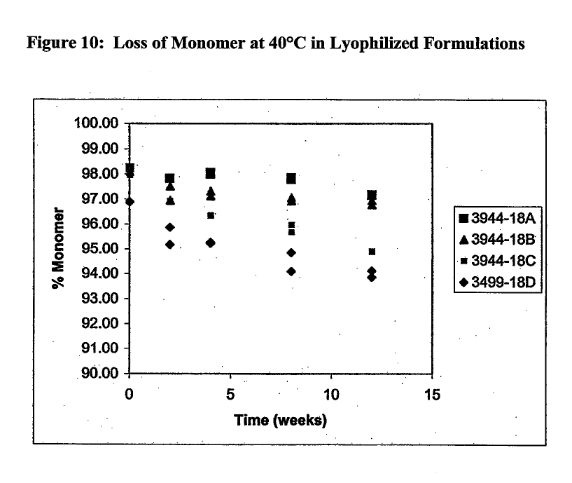

The reconstituted samples were analyzed by size exclusion chromatography for formation of both low molecular and high molecular weight degradation products. Low molecular weight degradation was apparent at some time points, but there did not seem to be a trend for formation. The amount at all time points was lower than 0.2% in any sample, which is considered the limit of detection for the assay by Biogenldec. The main pathway of degradation was through formation of dimer and higher order aggregates. The loss of monomer with time is shown in FIGS. 8, 9 and 10. At 40° C. storage there is some loss of monomer with the most rapid loss in Formulations 3944-18D and 3944-18C. Formulations 39944-18B and 3944-18A show a much less pronounced downward trend. The same order of trending is seen in the 30° C., with 3944-18D and 3944-18C showing a more rapid loss of monomer. None of the formulations show a loss of monomer at 5° C. for up to 6 months for 3944-18A and 3944-18D, and up to a year for 3944-18B and 3944-18C.

Selected samples were analyzed by cation exchange chromatography. There did not appear to be any differences among the formulations. At 5° C. there was a shift from 71% main peak to 66% main peak. The percent of acid peaks remained fairly constant over time, but the percent of basic peaks increased with time from 8-10% at the initial time point to 14-15% after one year. The 30 and 40° C. samples exhibited the same trend with the percent basic peak reaching 18-19% after one year at 30° C. and 22-26% after 3 months at 40° C. Additional work would be needed to characterize this reaction pathway and to determine the origin of the degradation species.

### Results of Study B

### Selection of Formulations

Study B was set up to study the effect of excipients in addition to sucrose on the stability of natalizumab and to verify results seen in Study A. Based on the results from Study A it was determined that a starting concentration of 40-50 mg/mL was optimal for both good cake formation and reconstitution characteristics. The protein to sugar ratio was fixed at 2:1 weight ratio, with an initial concentration of 50 mg/mL and the reconstitution concentration target was 200 mg/mL. In addition, one formulation was examined with a starting concentration of 40 mg/mL and a target reconstitution concentration of 160 mg/mL. This formulation also had a protein to sugar ratio of 1.6:1 weight ratio.

Formulation 3976-4C contained the same formulation as 3944-18C in Study A. The stability of the pre-lyophilized solution was examined in study A and not repeated for B. Reports from the literature have indicated that a low level of sodium chloride added to high concentration protein solutions could help reduce the viscosity of these solutions. Formulation 3976-4G had 15 mM NaCl added to the pre-lyophilization formula to examine the effect of NaCl. Polysorbate 20 (PS20) is frequently used in protein formulations instead of polysorbate 80 (PS80). In formulation 3976-4H, the 0.02% PS80 was replaced with an equal amount of PS20. In formulation 3976-4I, an equal amount of trehalose was substituted for the sucrose.

### Pre-Lyophilized Formulations

The pre-lyophilized formulations were analyzed for appearance. No significant changes to the appearance occurred for any of the solutions regardless of temperature of storage. They were all colorless and slightly opalescent with no appearance of particulates. There was no change in the protein concentration in any of the formulations. The turbidity was measured at all time points. For formulation 3976-4G, the initial turbidity measurement was high, but remained fairly constant at subsequent time points. No trend towards change in turbidity was observed in any of the formulations, although the value measured for 3976-4G continued to be higher at all temperatures than the other formulations.

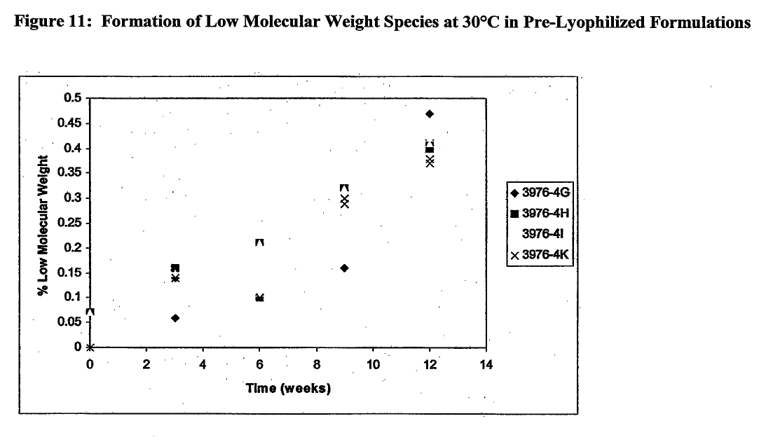

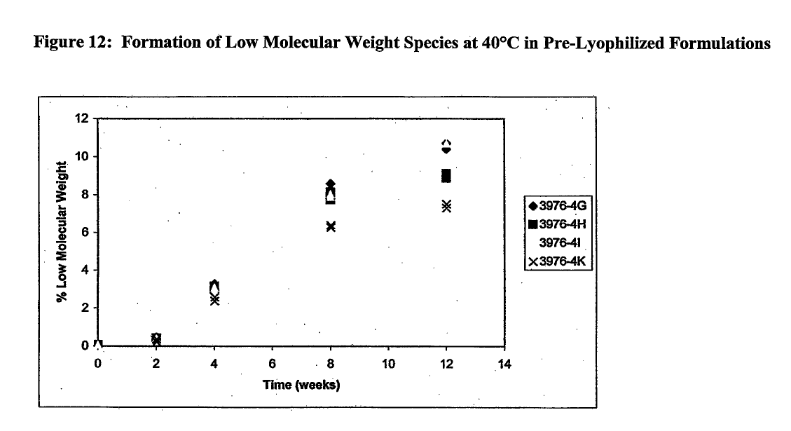

Loss of monomer due to formation of dimer and higher molecular weight aggregates and due to low molecular fragments was monitored. As seen previously, there was no change in the percent monomer for any of the samples up to 3 months at 5° C. Formulation 3976-4K was also analyzed at 6 months at 5° C. and showed no change in percent monomer. FIGS. 11 and 12 show the formation of low molecular weight species for each formulation at 30 and 40° C., respectively. As seen previously, there was substantial formation of low molecular weight species at 40° C. in all formulations with the rate of formation slightly lower in the 3976-4K, which had a lower protein concentration. These results were comparable to those seen in Study A. Otherwise there seemed to be no effect of excipients on this reaction. At 30° C., there was a slight rise in low molecular weight species. This rate was also comparable to that seen previously in Study 3A reaching between 0.2 to 0.4% after 12 weeks.

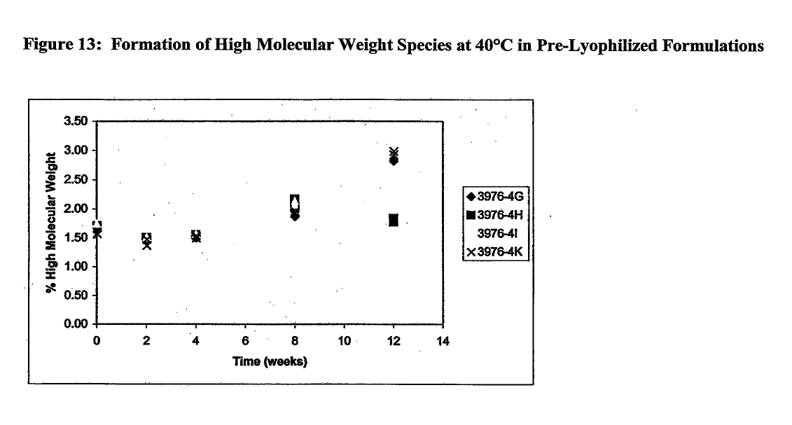

There was a slight increase in high molecular weight species at 40° C. for all the formulations except 3976-4H, which contained PS20 (FIG. 13). There was no change in high molecular weight for any formulations at the other temperatures. It appeared that all formulations were stable to formation of aggregate, and the major degradation pathway was formation of low molecular weight species at elevated temperature. Any of these formulations would be sufficiently stable for further development as a pre-lyophilized bulk drug.

Cation exchange chromatography was performed on all formulations at the initial time point, but only on 3976-4K after 6 months at 5° C. storage. As seen in Study A, there was a shift to more acidic species on storage. This degradation is driven by pH and temperature.

### Lyophilized Formulations

All samples were analyzed for moisture, cake appearance and reconstitution time at the initial time point. The cake appearance for all cakes was acceptable. The moisture levels were slightly lower than in Study A, generally between 3 and 4%, except for formulation 3976-4I, which had 5.4% moisture. Reconstitution time for all samples at all time points and temperatures were generally acceptable and below 10 minutes with a few exceptions taking between 10 and 14 minutes. Formulation 3976-4K, which had the lowest starting protein concentration also showed more rapid reconstitution times. Upon reconstitution all samples were colorless and slightly opalescent to opalescent for up to 12 weeks at all temperature with the exception of 3976-4I and 3976-4G. At 40° C. and 12 weeks, 3976-4I was very opalescent. Beginning at 6 weeks at 30° C. and 8 weeks for 5 and 40° C., 3976-4G was very opalescent. The addition of the NaCl did seem to reduce the viscosity of the formulation as seen by a tendency to less foaming and bubble formation during reconstitution, however, it also showed a significant increase in the opalescence of the solution. Formulation 3976-4C was analyzed at 6 months and 1 year at 5° C. At 5° C. after one year, 3976-4C showed a slight yellow color. Formulation 3976-4K was analyzed at 6 months and 1 year at 5 and 30° C. Formulation 3976-4K showed a slight yellow color at 30° C. after 6 months of storage, but not after 1 year at 5° C.

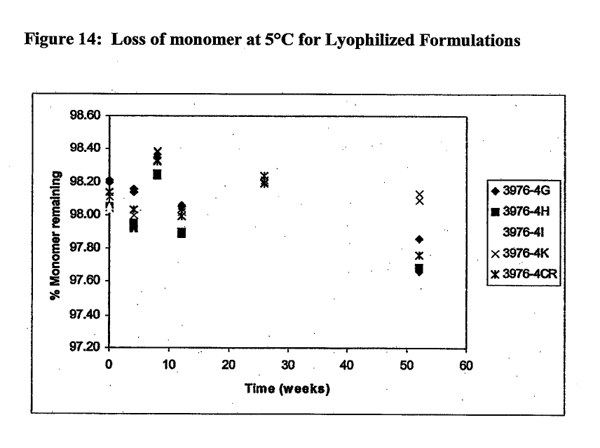

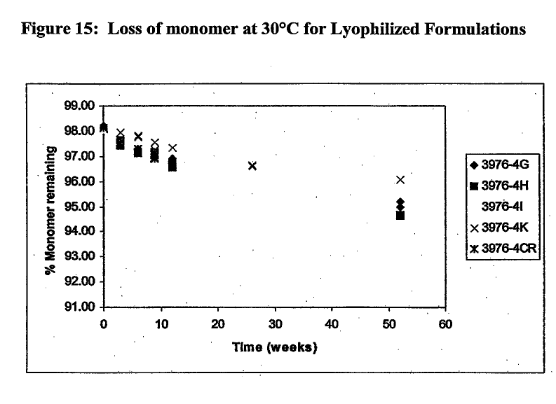

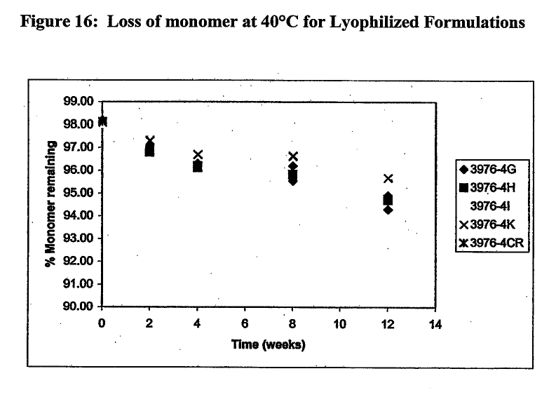

Samples were analyzed by size exclusion chromatography for up to 3 months at 40° C. At 5 and 30° C. samples were analyzed up to 3 months, and then an analysis was done at the one-year time point. In addition, formulations 3976-4C and 3976-4K were analyzed at 6 months as well. The amount of low molecular weight species was less than 0.2% in all samples. The major pathway of degradation was due to the formation of dimer and higher molecular weight species. FIGS. 14, 15 and 16 show the loss of monomer due to formation of aggregate for each temperature. It is apparent at all the temperatures that the loss of monomer in Formulation 3976-4I, which contains trehalose, is more rapid than the other formulations. The formulations containing PS20 and NaCl showed comparable degradation to the formulations containing sucrose with PS80. Formulation 3976-4K showed the least rapid loss of monomer at all temperatures. This formulation had a lower starting protein concentration, a lower reconstitution concentration and a higher sucrose to protein ratio.

Selected samples were analyzed by cation exchange chromatography. The results from this assay were somewhat variable, but all formulations appeared to remain stable to changes in charge distribution at 5° C. At 40° C. all formulations showed a decrease in the main peak and increases in both the acidic and basic species. This trend was also observed in the samples from Formulation 3976-4K, which were analyzed after 1 year storage at 30° C.

Formulation 3976-4K stored at 5 and 40° C. was analyzed at the 8 week time point in both the VCAM lysate and the Jurkat cell assays for potency. The results are shown in the Table.

TABLE 4
Potency Assay Results

| Sample/Assay | VCAM lysate | Jurkat |
|---|---|---|
| 3976-4K - 5° C. | 126% | 98.2 |

### Formation Screening Studies for Development of a High Concentration Lyophilized Formulation for Natalizumab

| | | |
|---|---|---|
| 3976-4K - 40° C. | 124.3% | 104% |

### Conclusions

### Pre-Lyophilized Bulk Formulation

In the high concentration liquid formulation there was no loss of monomer for up to 6 months at 5° C., however, there was an increase in acidic species probably due to deamidation. For 6 months at 30° C. and 3 months at 40° C., this formulation showed loss of monomer through formation of both high and low molecular weight degradation species. A high protein concentration liquid in the current formulation may not exhibit sufficient long-term stability to be suitable as a commercial formulation without further optimization.

None of the pre-lyophilized formulations showed any significant loss of monomer when stored at 5° C. for 3 to 6 months. The change in the amount of deamidation for these formulations was comparable to that seen in the high concentration liquid. The formulations stored at 30° C. showed little or no increase in dimer and high molecular weight aggregates for up to 3 months, however there was an increase in the amount of low molecular weight fragmentation species formed. At 40° C., the increase in low molecular weight species was more rapid than the increase in high molecular weight species. The rate of increase of the low molecular weight species appeared to be correlated with the protein concentration.

In general, formulations containing a protein concentration of 50 mg/mL or less with histidine, sucrose, and PS80 appeared to demonstrate sufficient stability for pre-lyophilized bulk.

### Lyophilized Formulations

Formulations that were lyophilized from 50 mg/mL or less and reconstituted to 100 to 200 mg/mL showed good cake formation and acceptable reconstitution times at all temperatures and time points. Formulations containing histidine, sucrose, and PS80 had no loss of monomer up to one year at 5° C. Some of these formulations showed a slight increase in basic species after storage for a year; however, this increase is hard to quantitate given the variability in the assay. The major route of degradation of the protein at elevated temperatures was formation of dimer and higher molecular weight aggregates. Addition of NaCl decreased the viscosity but resulted in increased turbidity of the solution. Substitution of PS20 for PS80 appeared to have no effect on the stability, while substitution of trehalose for sucrose increased the rate of formation of aggregate.

A lyophilized formulation containing sucrose, histidine, and polysorbate 80 with natalizumab demonstrates sufficient stability to move into pre-clinical and early clinical studies. Additional studies will be performed to optimize the starting protein concentration, the sucrose to protein ratio, the reconstituted protein concentration and the lyophilization cycle. In addition the stability of the reconstituted samples will be examined.

TABLE 5
Pre-Lyo
20 mg/mL Natalizumab, 20 mg/mL Sucrose, 6 mM Histidine, 0.02% PS-80
Manufactured:
Testing Start Date: 0.5 mL fill

| Time | | | | | | | | | | % Acidic |
|---|---|---|---|---|---|---|---|---|---|---|
| Point/ | | | Conc. | Turbidity | % | % | % | % | SEC Ref Std & | % Main |
| Temp. | Vial | Appearance | (mg/mL) | A360 nm | Monomer | Dimer | Aggregate | LMW | Avg. area count | % Basic |
| 0 | 1 | N/A | 21.0 | 0.041 | 98.29 | 1.01 | 0.63 | 0.06 | RS007-001 | 18.1 |
| | | | | | | | | | 3448 | 69.6 |
| | | | | | | | | | | 12.4 |
| | 2 | N/A | 21.0 | | | 1.02 | 0.63 | 0.06 | | 18.6 |
| | | | | | | | | | | 70.6 |
| | | | | | | | | | | 10.9 |
| 4 wk, | 1 | clear, v. sl opal, | N/A | 0.035 | 98.43 | 0.99 | 0.49 | 0.09 | RS007-001 | N/A |
| 5 C. | | colorless | | | | | | | 33690 | |
| | 2 | clear, v. sl opal, | | 0.037 | 98.44 | 0.98 | 0.49 | 0.09 | | |
| | | colorless | | | | | | | | |
| 8 wk, | 1 | clear, colorless | N/A | 0.021 | 98.43 | 0.98 | 0.47 | 0.11 | RS007-001 | N/A |
| 5 C. | 2 | clear, colorless | | 0.020 | 98.50 | 0.97 | 0.45 | 0.07 | 33245 | |
| 12 wk, | 1 | clear, colorless | 20.7 | 0.025 | 98.48 | 1.01 | 0.42 | 0.09 | RS007-001 | N/A |
| 5 C. | 2 | clear, colorless | 20.9 | 0.027 | 98.48 | 1.01 | 0.43 | 0.08 | 33881 | |
| 6 mo, | 1 | clear, colorless | 20.5 | 0.035 | 98.49 | 1.01 | 0.36 | 0.14 | REF025 | 24.0 |
| 5 C. | | | | | | | | | (RS007-001) | 63.9 |
| | | | | | | | | | 33759 | 63.9 |
| | 2 | clear, colorless | 20.5 | 0.033 | 98.47 | 1.01 | 0.37 | 0.15 | | 23.9 |
| | | | | | | | | | | 63.9 |
| | | | | | | | | | | 12.2 |

30° C.

| | | | | | | | | | | |
|---|---|---|---|---|---|---|---|---|---|---|
| 0 | 1 | N/A | 21.0 | 0.041 | 98.29 | 1.01 | 0.63 | 0.06 | RS007-001 | 18.1 |
| | | | | | | | | | 3448 | 69.6 |
| | | | | | | | | | | 12.4 |
| | 2 | N/A | 21.0 | | 98.30 | 1.02 | 0.63 | 0.06 | | 18.6 |
| | | | | | | | | | | 70.6 |
| | | | | | | | | | | 10.9 |
| 3 wk, | 1 | clear, v. sl opal, | N/A | 0.029 | 98.46 | 0.93 | 0.48 | 0.13 | RS007-001 | N/A |
| 30 C. | | colorless | | | | | | | 33707 | |
| | 2 | clear, v. sl opal, | | 0.044 | 98.48 | 0.93 | 0.48 | 0.12 | | |
| | | colorless | | | | | | | | |
| 6 wk, | 1 | clear, colorless | N/A | 0.028 | 98.40 | 0.94 | 0.49 | 0.17 | RS007-001 | N/A |
| 30 C. | 2 | clear, colorless | | 0.027 | 98.42 | 0.95 | 0.48 | 0.16 | 33758 | |
| 9 wk, | 1 | clear, colorless | N/A | 0.034 | 98.45 | 0.93 | 0.43 | 0.19 | RS007-001 | N/A |
| 30 C. | 2 | clear, colorless | | 0.031 | 98.45 | 0.93 | 0.43 | 0.19 | 33757 | |
| 12 wk, | 1 | clear, colorless | 21.0 | 0.018 | 98.37 | 0.99 | 0.39 | 0.24 | RS007-001 | N/A |
| 30 C. | 2 | clear, colorless | 20.8 | 0.024 | 98.38 | 0.99 | 0.40 | 0.23 | 33897 | |

40° C.

| | | | | | | | | | | |
|---|---|---|---|---|---|---|---|---|---|---|
| 0 | 1 | N/A | 21.0 | 0.041 | 98.29 | 1.01 | 0.63 | 0.06 | RS007-001 | 18.1 |
| | | | | | | | | | 3448 | 69.6 |
| | | | | | | | | | | 12.4 |
| | 2 | 21.0 | 98.30 | | 0.63 | 0.06 | 21.0 | 98.30 | | 18.6 |
| | | | | | | | | | | 70.6 |
| | | | | | | | | | | 10.9 |
| 2 wk, | 1 | clear, v. sl opal, | N/A | 0.060 | 98.33 | 0.96 | 0.49 | 0.22 | RS007-001 | N/A |
| 40 C. | | colorless | | | | | | | 33834 | |
| | 2 | clear, v. sl opal, | | 0.045 | 98.34 | 0.95 | 0.49 | 0.22 | | |
| | | colorless | | | | | | | | |
| 4 wk, | 1 | clear, v. sl opal, | N/A | 0.040 | 98.20 | 1.00 | 0.45 | 0.35 | RS007-001 | N/A |
| 40 C. | | colorless | | | | | | | 33690 | |
| | 2 | clear, v. sl opal, | | 0.043 | 98.03 | 1.04 | 0.49 | 0.44 | | |
| | | colorless | | | | | | | | |
| 8 wk, | 1 | clear, colorless | N/A | 0.060 | 93.93 | 1.61 | 0.65 | 3.81 | 33693 | N/A |
| 40 C. | 2 | clear, colorless | | 0.057 | 94.15 | 1.57 | 0.64 | 3.63 | | |
| 12 wk, | 1 | clear, colorless | 20.8 | 0.068 | 91.12 | 2.01 | 1.04 | 5.83 | RS007-001 | 63.6 |
| 40 C. | | | | | | | | | 33891 | 20.7 |
| | | | | | | | | | | 15.8 |
| | 2 | clear, colorless | 20.5 | 0.055 | 92.00 | 1.76 | 1.03 | 5.22 | | 64.7 |
| | | | | | | | | | | 19.2 |
| | | | | | | | | | | 16.1 |

TABLE 6
Pre-Lyo
40 mg/mL Natalizumab, 40 mg/mL Sucrose, 6 mM Histidine, 0.02% PS-80
Manufactured:
Testing Start Date: 0.5 mL fill

| Time | | | | | | | | | | % Acidic |
|---|---|---|---|---|---|---|---|---|---|---|
| Point/ | | | Conc. | Turbidity | % | % | % | % | SEC Ref Std & | % Main |
| Temp. | Vial | Appearance | (mg/mL) | A360 nm | Monomer | Dimer | Aggregate | LMW | Avg. area count | % Basic |
| 0 | 1 | NIA | 40.1 | 0.053 | 98.26 | 1.02 | 0.65 | 0.07 | RS007-001 | 19.0 |
| | | | | | | | | | 3448 | 70.9 |
| | | | | | | | | | | 10.0 |
| | 2 | N/A | 40.1 | | 98.26 | 1.02 | 0.64 | 0.07 | | 18.5 |
| | | | | | | | | | | 71.1 |
| | | | | | | | | | | 10.4 |
| 4 wk, | 1 | clear, sl opal, | N/A | 0.054 | 98.38 | 0.99 | 0.52 | 0.10 | RS007-001 | N/A |
| 5 C. | | colorless | | | | | | | 33690 | |
| | 2 | clear, sl opal, | | 0.054 | 98.37 | 0.99 | 0.53 | 0.11 | | |
| | | colorless | | | | | | | | |
| 8 wk, | 1 | sl opal, colorless | N/A | 0.050 | 98.42 | 1.01 | 0.48 | 0.09 | RS007-001 | N/A |
| 5 C. | 2 | sl opal, colorless | | 0.038 | 98.44 | 0.98 | 0.46 | 0.11 | 33245 | |
| 12 wk, | 1 | v. sl opal, colorless | 39.3 | 0.059 | 98.43 | 1.05 | 0.42 | 0.10 | RS007-001 | 21.4 |
| 5 C. | | | | | | | | | 338881 | 69.1 |
| | | | | | | | | | | 9.4 |
| | 2 | v. sl opal, colorless | 39.5 | 0.043 | 98.45 | 1.02 | 0.44 | 0.09 | | 21.5 |
| | | | | | | | | | | 70.0 |
| | | | | | | | | | | 8.4 |
| 6 mo, | 1 | v. sl opal, colorless | 39.1 | 0.055 | 98.47 | 1.01 | 0.37 | 0.15 | REF025 | 25.2 |
| 5 C. | | | | | | | | | (RS007-001) | 62.8 |
| | | | | | | | | | 33759 | 11.9 |
| | 2 | v. sl opal, colorless | 39.5 | 0.052 | 98.71 | 0.95 | 0.34 | — | | 25.5 |
| | | | | | | | | | | 63.0 |
| | | | | | | | | | | 11.5 |

30° C.

| | | | | | | | | | | |
|---|---|---|---|---|---|---|---|---|---|---|
| 0 | 1 | NIA | 40.1 | 0.053 | 98.26 | 1.02 | 0.65 | 0.07 | RS007-001 | 19.0 |
| | | | | | | | | | 3448 | 70.9 |
| | | | | | | | | | | 10.0 |
| | 2 | N/A | 40.1 | | 98.26 | 1.02 | 0.64 | 0.07 | | 18.5 |
| | | | | | | | | | | 71.1 |
| | | | | | | | | | | 10.4 |
| 3 wk, | 1 | clear, v. sl opal, | N/A | 0.042 | 98.45 | 0.95 | 0.46 | 0.13 | RS007-001 | N/A |
| 30 C. | | colorless | | | | | | | 33707 | |
| | 2 | clear, v. sl opal, | | 0.055 | 98.45 | 0.95 | 0.46 | 0.13 | | |
| | | colorless | | | | | | | | |
| 6 wk, | 1 | clear, colorless | N/A | 0.056 | 98.41 | 0.96 | 0.47 | 0.17 | RS007-001 | N/A |
| 30 C. | 2 | clear, colorless | | 0.053 | 98.42 | 0.96 | 0.46 | 0.16 | 33758 | |
| 9 wk, | 1 | sl opal, colorless | N/A | 0.058 | 98.38 | 0.99 | 0.41 | 0.22 | RS007-001 | N/A |
| 30 C. | 2 | sl opal, colorless | | 0.060 | 98.40 | 0.98 | 0.40 | 0.22 | 33757 | |
| 12 wk, | 1 | v. sl opal, colorless | 39.6 | 0.042 | 98.33 | 1.04 | 0.36 | 0.26 | RS007-001 | 46.3 |
| 30 C. | | | | | | | | | 33897 | 8.0 |
| | | | | | | | | | | 45.7 |
| | 2 | v. sl opal, colorless | 40.7 | 0.042 | 98.33 | 1.03 | 0.37 | 0.26 | | 45.8 |
| | | | | | | | | | | 46.5 |
| | | | | | | | | | | 7.7 |

40° C.

| | | | | | | | | | | |
|---|---|---|---|---|---|---|---|---|---|---|
| 0 | 1 | NIA | 40.1 | 0.053 | 98.26 | 1.02 | 0.65 | 0.07 | RS007-001 | 19.0 |
| | | | | | | | | | 3448 | 70.9 |
| | | | | | | | | | | 10.0 |
| | 2 | N/A | 40.1 | | 98.26 | 1.02 | 0.64 | 0.07 | | 18.5 |
| | | | | | | | | | | 71.1 |
| | | | | | | | | | | 10.4 |
| 2 wk, | 1 | clear, v. sl opal, | N/A | 0.071 | 98.30 | 0.97 | 0.48 | 0.25 | RS007-001 | N/A |
| 40 C. | | colorless | | | | | | | 33834 | |
| | 2 | clear, v. sl opal, | | 0.062 | 98.30 | 0.97 | 0.48 | 0.25 | | |
| | | colorless | | | | | | | | |
| 4 wk, | 1 | clear, sl opal, | N/A | 0.065 | 97.92 | 1.04 | 0.47 | 0.58 | RS007-001 | N/A |
| 40 C. | | colorless | | | | | | | 33690 | |
| | 2 | clear, sl opal, | | 0.065 | 97.90 | 1.05 | 0.47 | 0.58 | | |
| | | colorless | | | | | | | | |
| 8 wk, | 1 | sl opal, colorless | N/A | 0.084 | 93.68 | 1.41 | 0.68 | 4.22 | RS007-001 | N/A |
| 40 C. | 2 | sl opal, colorless | | 0.080 | 93.56 | 1.36 | 0.72 | 4.36 | 33693 | |
| 12 wk, | 1 | v. sl opal, colorless | 38.8 | 0.100 | 89.97 | 1.76 | 1.20 | 7.07 | RS007-001 | 69.2 |
| 40 C. | | | | | | | | | 33891 | 16.5 |
| | | | | | | | | | | 14.4 |
| | 2 | v. sl opal, colorless | 39.1 | 0.103 | 90.02 | 1.83 | 1.47 | 6.69 | | 69.2 |
| | | | | | | | | | | 17.2 |
| | | | | | | | | | | 13.6 |

TABLE 7
Pre-Lyo
40 mg/mL Natalizumab, 40 mg/mL Sucrose, 6 mM Histidine, 0.02% PS-80
Manufactured:
Testing Start Date: 0.5 mL fill

| Time | | | | | | | | | | % Acidic |
|---|---|---|---|---|---|---|---|---|---|---|
| Point/ | | | Conc. | Turbidity | % | % | % | % | SEC Ref Std & | % Main |
| Temp. | Vial | Appearance | (mg/mL) | A360 nm | Monomer | Dimer | Aggregate | LMW | Avg. area count | % Basic |
| 0 | 1 | N/A | 52.1 | 0.074 | 98.19 | 1.03 | 0.71 | 0.07 | RS007-001 | 18.6 |
| | | | | | | | | | 3448 | 71.0 |
| | | | | | | | | | | 10.4 |
| | 2 | N/A | 51.5 | | 98.18 | 1.03 | 0.71 | 0.08 | | 18.4 |
| | | | | | | | | | | 71.2 |
| | | | | | | | | | | 10.4 |
| 4 wk, | 1 | clear, sl opal, | N/A | 0.070 | 98.31 | 1.01 | 0.57 | 0.10 | RS007-001 | N/A |
| 5 C. | | colorless | | | | | | | 33690 | |
| | 2 | clear, sl opal, | | 0.068 | 98.33 | 1.01 | 0.56 | 0.10 | | |
| | | colorless | | | | | | | | |
| 8 wk, | 1 | sl opal, colorless | N/A | 0.062 | 98.40 | 1.00 | 0.50 | 0.09 | RS007-001 | N/A |
| 5 C. | 2 | sl opal, colorless | | 0.056 | 98.37 | 1.01 | 0.52 | 0.10 | 33245 | |
| 12 wk, | 1 | sl opal, colorless | 50.3 | 0.054 | 98.42 | 1.04 | 0.45 | 0.09 | RS007-001 | 21.9 |
| 5 C. | | | | | | | | | 33881 | 66.2 |
| | | | | | | | | | | 11.9 |
| | 2 | sl opal, colorless | 50.6 | 0.057 | 98.41 | 1.04 | 0.46 | 0.09 | | 22.2 |
| | | | | | | | | | | 68.2 |
| | | | | | | | | | | 9.6 |
| 6 mo, | 1 | sl opal, colorless | 51.9 | 0.064 | 98.44 | 1.03 | 0.38 | 0.14 | REF025 | 26.1 |
| 5 C. | | | | | | | | | (RS007-001) | 62.2 |
| | | | | | | | | | 33759 | 11.7 |
| | 2 | sl opal, colorless | 50.6 | 0.060 | 98.44 | 1.03 | 0.38 | 0.15 | | 26.1 |
| | | | | | | | | | | 61.9 |
| | | | | | | | | | | 12.0 |

30° C.

| | | | | | | | | | | |
|---|---|---|---|---|---|---|---|---|---|---|
| 0 | 1 | N/A | 52.1 | 0.074 | 98.19 | 1.03 | 0.71 | 0.07 | RS007-001 | 18.6 |
| | | | | | | | | | 3448 | 71.0 |
| | | | | | | | | | | 10.4 |
| | 2 | N/A | 51.5 | | 98.18 | 1.03 | 0.71 | 0.08 | | 18.4 |
| | | | | | | | | | | 71.2 |
| | | | | | | | | | | 10.4 |
| 3 wk, | 1 | clear, sl opal, | N/A | 0.057 | 98.42 | 0.96 | 0.49 | 0.13 | RS007-001 | N/A |
| 30 C. | | colorless | | | | | | | 33707 | |
| | 2 | clear, sl opal, | | 0.058 | 98.40 | 0.98 | 0.49 | 0.14 | | |
| | | colorless | | | | | | | | |
| 6 wk, | 1 | clear, colorless | N/A | 0.059 | 98.55 | 0.93 | 0.44 | 0.08 | RS007-001 | N/A |
| 30 C. | 2 | clear, colorless | | 0.067 | 98.37 | 0.99 | 0.46 | 0.17 | 33758 | |
| 9 wk, | 1 | sl opal, colorless | N/A | 0.064 | 98.34 | 0.99 | 0.41 | 0.26 | RS007-001 | N/A |
| 30 C. | 2 | sl opal, colorless | | 0.063 | 98.35 | 0.99 | 0.41 | 0.25 | 33757 | |
| 12 wk, | 1 | sl opal, colorless | 52.6 | 0.052 | 98.21 | 1.05 | 0.37 | 0.36 | RS007-001 | 48.5 |
| 30 C. | | | | | | | | | 33897 | 44.4 |
| | | | | | | | | | | 7.1 |
| | 2 | sl opal, colorless | 50.1 | 0.055 | 98.21 | 1.06 | 0.37 | 0.36 | | 48.2 |
| | | | | | | | | | | 44.2 |
| | | | | | | | | | | 7.6 |

40° C.

| | | | | | | | | | | |
|---|---|---|---|---|---|---|---|---|---|---|
| 0 | 1 | N/A | 52.1 | 0.074 | 98.19 | 1.03 | 0.71 | 0.07 | RS007-001 | 18.6 |
| | | | | | | | | | 3448 | 71.0 |
| | | | | | | | | | | 10.4 |
| | 2 | N/A | 51.5 | | 98.18 | 1.03 | 0.71 | 0.08 | | 18.4 |
| | | | | | | | | | | 71.2 |
| | | | | | | | | | | 10.4 |
| 2 wk, | 1 | clear, sl opal, | N/A | 0.081 | 98.16 | 1.01 | 0.48 | 0.35 | RS007-001 | N/A |
| 40 C. | | colorless | | | | | | | 33834 | |
| | 2 | clear, sl opal, | | 0.080 | 98.18 | 0.99 | 0.48 | 0.35 | | |
| | | colorless | | | | | | | | |
| 4 wk, | 1 | clear, sl opal, | N/A | 0.071 | 95.11 | 1.08 | 0.47 | 3.34 | RS007-001 | N/A |
| 40 C. | | colorless | | | | | | | 33690 | |
| | 2 | clear, sl opal, | | 0.080 | 95.33 | 1.08 | 0.48 | 3.11 | | |
| | | colorless | | | | | | | | |
| 8 wk, | 1 | sl opal, colorless | NIA | 0.095 | 91.46 | 1.36 | 0.89 | 6.29 | RS007-001 | N/A |
| 40 C. | 2 | sl opal, colorless | | 0.095 | 91.44 | 1.35 | 0.89 | 6.33 | 33693 | |
| 12 wk, | 1 | sl opal, colorless | 49.8 | 0.116 | 86.90 | 1.64 | 1.86 | 9.60 | RS007-001 | 73.7 |
| 40 C. | | | | | | | | | 33891 | 14.8 |
| | | | | | | | | | | 11.5 |

(v. = very, sl = slightly, opal = opalescent)

TABLE 8
Pre-Lyo
75 mg/mL Natalizumab, 40 mg/mL Sucrose, 6 mM Histidine, 0.02% PS-80
Manufactured:
Testing Start Date: 0.5 mL fill

| | | | | | | | | | SEC Ref | % Acidic |
|---|---|---|---|---|---|---|---|---|---|---|
| Time | | | Conc. | Turbidity | % | % | | % | Std & Avg. | % Main |
| Point/Temp | Vial | Appearance | (mg/mL) | A360 nm | Monomer | Dimer | % Aggregate | LMW | area count | % Basic |
| 0 | 1 | N/A | 77.2 | 0.097 | 97.23 | 1.53 | 1.15 | 0.09 | RS007-001 | 18.5 |
| | | | | | | | | | 3448 | 72.3 |
| | | | | | | | | | | 9.2 |
| | 2 | N/A | 77.6 | | 97.23 | 1.53 | 1.14 | 0.09 | | 17.9 |
| | | | | | | | | | | 71.9 |
| | | | | | | | | | | 10.1 |
| 4 wk, 5 C. | 1 | clear, sl opal, | N/A | 0.089 | 97.46 | 1.51 | 0.90 | 0.13 | RS007-001 | N/A |
| | | colorless | | | | | | | 33690 | |
| | 2 | clear, sl opal, | | 0.089 | 97.47 | 1.51 | 0.89 | 0.12 | | |
| | | colorless | | | | | | | | |
| 8 wk, 5 C. | 1 | sl opal, | N/A | 0.071 | 97.54 | 1.51 | 0.83 | 0.13 | RS007-001 | N/A |
| | | colorless | | | | | | | 33245 | |
| | 2 | sl opal, | | 0.071 | 97.55 | 1.51 | 0.81 | 0.12 | | |
| | | colorless | | | | | | | | |
| 12 wk, 5 C. | 1 | sl opal, | 73.1 | 0.072 | 97.58 | 1.56 | 0.75 | 0.11 | RS007-001 | N/A |
| | | colorless | | | | | | | 33881 | |
| | 2 | sl opal, | 75.4 | 0.070 | 97.58 | 1.56 | 0.75 | 0.11 | | |
| | | colorless | | | | | | | | |
| 6 mo, 5 C. | 1 | sl opal, | 75.9 | 0.081 | 97.72 | 1.53 | 0.59 | 0.15 | REF025 | 26.2 |
| | | colorless | | | | | | | (RS007- | 62.0 |
| | | | | | | | | | 001) 33759 | 11.8 |
| | 2 | sl opal, | 75.9 | 0.078 | 97.73 | 1.53 | 0.59 | 0.15 | | 25.9 |
| | | colorless | | | | | | | | 62.4 |
| | | | | | | | | | | 11.7 |

30° C.

| | | | | | | | | | | |
|---|---|---|---|---|---|---|---|---|---|---|
| 0 | 1 | N/A | 77.2 | 0.097 | 97.23 | 1.53 | 1.15 | 0.09 | RS007-001 | 18.5 |
| | | | | | | | | | 3448 | 72.3 |
| | | | | | | | | | | 9.2 |
| | 2 | N/A | 77.6 | | 97.23 | 1.53 | 1.14 | 0.09 | | 17.9 |
| | | | | | | | | | | 71.9 |
| | | | | | | | | | | 10.1 |
| 3 wk, 30 C. | 1 | clear, sl opal, | N/A | 0.071 | 97.65 | 1.44 | 0.73 | 0.17 | RS007-001 | N/A |
| | | colorless | | | | | | | 33707 | |
| | 2 | clear, sl opal, | | 0.089 | 97.67 | 1.44 | 0.73 | 0.17 | | |
| | | colorless | | | | | | | | |
| 6 wk, 30 C. | 1 | v. sl opal, | N/A | 0.077 | 97.64 | 1.45 | 0.70 | 0.21 | RS007-001 | N/A |
| | | colorless | | | | | | | 33758 | |
| | 2 | v. sl opal, | | 0.070 | 97.63 | 1.45 | 0.70 | 0.21 | | |
| | | colorless | | | | | | | | |
| 9 wk, 30 C. | 1 | sl opal, | N/A | 0.084 | 97.90 | 1.36 | 0.56 | 0.18 | RS007-001 | N/A |
| | | colorless | | | | | | | 33757 | |
| | 2 | sl opal, | | 0.083 | 97.91 | 1.36 | 0.56 | 0.17 | | |
| | | colorless | | | | | | | | |
| 12 wk, 30 C. | 1 | sl opal, | 79.7 | 0.072 | 97.42 | 1.53 | 0.55 | 0.51 | RS007-001 | N/A |
| | | colorless | | | | | | | 33897 | |
| | 2 | sl opal, | 75.6 | 0.081 | 97.41 | 1.53 | 0.54 | 0.52 | | |
| | | colorless | | | | | | | | |

40° C.

| | | | | | | | | | | |
|---|---|---|---|---|---|---|---|---|---|---|
| 0 | 1 | N/A | 77.2 | 0.097 | 97.23 | 1.53 | 1.15 | 0.09 | RS007-001 | 18.5 |
| 0 | 1 | N/A | 77.2 | 0.097 | 97.23 | 1.53 | 1.15 | 0.09 | RS007-001 | 18.5 |
| | | | | | | | | | 3448 | 72.3 |
| | | | | | | | | | | 9.2 |
| | 2 | N/A | 77.6 | | 97.23 | 1.53 | 1.14 | 0.09 | | 17.9 |
| | | | | | | | | | | 71.9 |
| | | | | | | | | | | 10.1 |
| 2 wk, 40 C. | 1 | clear, sl opal, | N/A | 0.092 | 97.31 | 1.46 | 0.73 | 0.51 | RS007-001 | N/A |
| | | colorless | | | | | | | 33834 | |
| | 2 | clear, sl opal, | | 0.111 | 97.33 | 1.46 | 0.72 | 0.50 | | |
| | | colorless | | | | | | | | |
| 4 wk, 40 C. | 1 | clear, sl opal, | N/A | 0.104 | 93.12 | 1.53 | 0.70 | 4.66 | RS007-001 | N/A |
| | | colorless | | | | | | | 33690 | |
| | 2 | clear, sl opal, | | 0.098 | 93.00 | 1.51 | 0.71 | 4.79 | | |
| | | colorless | | | | | | | | |
| 8 wk, 40 C. | 1 | sl opal, colorless | N/A | 0.136 | 88.74 | 1.83 | 1.37 | 8.06 | RS007-001 | N/A |
| | 2 | sl opal, colorless | | 0.135 | 88.57 | 1.80 | 1.33 | 8.29 | 33498 | |
| 12 wk, 40 C. | 1 | sl opal, colorless | 79.4 | 0.165 | 83.49 | 2.09 | 2.85 | 11.57 | RS07-001 | 68.2 |
| | | | | | | | | | 33891 | 17.1 |
| | | | | | | | | | | 14.6 |
| | 2 | sl opal, colorless | 84.1 | 0.174 | 86.55 | 1.26 | 1.78 | 10.41 | | 68.6 |
| | | | | | | | | | | 13.7 |
| | | | | | | | | | | 17.7 |

TABLE 9
Reconstituted
100 mg/mL Natalizumab, 100 mg/mL Sucrose, 30 mM Histidine, 0.1% PS-80
Manufactured:
Testing Start Date: 0.5 mL fill

| Time | | | Recon | | | | | | | | | % Acidic |
|---|---|---|---|---|---|---|---|---|---|---|---|---|
| Point/ | | | Time | KF % | Conc. | Turbidity | % | % | % | % | SEC Ref Std & | % Main |
| Temp. | Vial | Appearance | (min.) | Moisture | (mg/mL) | A360 nm | Monomer | Dimer | Aggregate | LMW | Avg. area count | % Basic |
| 0 | 1 | clear, sl | 4.0 | 4.7 | 95.7 | 0.111 | 98.24 | 0.97 | 0.78 | — | RS007-001 | 19.1 70.8 |
| | | opal | | | | | | | | | 3448 | 10.1 |
| | 2 | clear, sl | 3.5 | | 96.7 | 0.100 | 98.23 | 0.98 | 0.79 | — | | 19.3 72.2 |
| | | opal | | | | | | | | | | 8.6 |
| 4 wk, | 1 | clear, sl | 5.1 | N/A | N/A | 0.146 | 98.41 | 0.90 | 0.70 | — | RS007-001 | N/A |
| 5 C. | | opal | | | | | | | | | 33720 | |
| | 2 | clear, sl | 5.2 | | | 0.164 | 98.41 | 0.89 | 0.69 | — | | |
| | | opal | | | | | | | | | | |
| 8 wk, | 1 | opal, | 4.4 | N/A | N/A | 0.136 | 98.40 | 0.91 | 0.69 | — | RS007-001 | N/A |
| 5 C. | | colorless | | | | | | | | | 33245 | |
| | 2 | opal, | 6.1 | | | 0.138 | 98.41 | 0.91 | 0.68 | | | |
| | | colorless | | | | | | | | | | |
| 12 wk, | 1 | opal, | 3.2 | N/A | 94.2 | 0.135 | 98.04 | 1.10 | 0.77 | 0.09 | RS007-001 | N/A |
| 5 C. | | colorless | | | | | | | | | 33881 | |
| | 2 | opal, | 5.0 | | 96.1 | 0.134 | 98.07 | 1.08 | 0.76 | 0.08 | | |
| | | colorless | | | | | | | | | | |
| 6 mo, | 1 | opal, | 6.9 | N/A | 96.3 | 0.150 | 98.35 | 1.01 | 0.64 | — | REF025 | 21.1 |
| 5 C. | | colorless | | | | | | | | | (RS007-001) | 66.8 |
| | | | | | | | | | | | 33759 | 12.1 |
| | 2 | opal, | 6.2 | | 96.2 | 0.153 | 98.33 | 1.02 | 0.66 | — | | 21.2 |
| | | colorless | | | | | | | | | | 66.6 |
| | | | | | | | | | | | | 12.2 |

30° C.

| | | | | | | | | | | | | |
|---|---|---|---|---|---|---|---|---|---|---|---|---|
| 0 | 1 | clear, sl | 4.0 | 4.7 | 95.7 | 0.111 | 98.24 | 0.97 | 0.78 | — | RS007-001 | 19.1 |
| | | opal | | | | | | | | | 3448 | 70.8 |
| | | | | | | | | | | | | 10.1 |
| | 2 | clear, sl | 3.5 | | 96.7 | 0.100 | 98.23 | 0.98 | 0.79 | — | | 19.3 |
| | | opal | | | | | | | | | | 72.2 |
| | | | | | | | | | | | | 8.6 |
| 3 wk, | 1 | clear, sl | 3.5 | N/A | N/A | 0.143 | 98.35 | 0.98 | 0.53 | — | RS007-001 | N/A |
| 30 C. | | opal, | | | | | | | | | 33707 | |
| | | colorless | | | | | | | | | | |
| | 2 | clear, sl | 6.0 | | | 0.151 | 98.36 | 0.98 | 0.51 | — | | |
| | | opal, | | | | | | | | | | |
| | | colorless | | | | | | | | | | |
| 6 wk, | 1 | sl opal, | 3.2 | N/A | N/A | 0.148 | 98.44 | 1.03 | 0.54 | — | RS007-001 | N/A |
| 30 C. | | colorless | | | | | | | | | 33758 | |
| | 2 | sl opal, | 4.0 | | | 0.132 | 98.42 | 1.04 | 0.54 | — | | |
| | | colorless | | | | | | | | | | |
| 9 wk, | 1 | opal, | 5.1 | N/A | N/A | 0.156 | 98.22 | 1.12 | 0.65 | — | RS007-001 | N/A |
| 30 C. | | colorless | | | | | | | | | 33757 | |
| | 2 | opal, | 6.3 | | | 0.161 | 98.24 | 1.10 | 0.66 | — | | |
| | | colorless | | | | | | | | | | |
| 12 wk, | 1 | opal, | 4.5 | N/A | 97.6 | 0.142 | 97.85 | 1.36 | 0.73 | 0.07 | RS007-001 | N/A |
| 30 C. | | colorless | | | | | | | | | 33897 | |
| | 2 | opal, | 3.3 | | 98.6 | 0.140 | 97.86 | 1.35 | 0.72 | 0.07 | | |
| | | colorless | | | | | | | | | | |
| 6 mo, | 1 | opal, | 4.2 | N/A | 96.0 | 0.169 | 97.86 | 1.87 | 0.27 | — | REF025 | 21.6 |
| 30 C. | | colorless | | | | | | | | | (RS007-001) | 64.0 |
| | | | | | | | | | | | 33774 | 14.4 |
| | 2 | opal, | 3.6 | | 97.3 | 0.175 | 97.83 | 1.53 | 0.64 | — | | 21.6 |
| | | colorless | | | | | | | | | | 64.4 |
| | | | | | | | | | | | | 14.0 |

40° C.

| | | | | | | | | | | | | |
|---|---|---|---|---|---|---|---|---|---|---|---|---|
| 0 | 1 | clear, sl | 4.0 | 4.7 | 95.7 | 0.111 | 98.24 | 0.97 | 0.78 | — | RS007-001 | 19.1 |
| | | opal | | | | | | | | | 3448 | 70.8 |
| | | | | | | | | | | | | 10.1 |
| | 2 | clear, sl | 3.5 | | 96.7 | 0.100 | 98.23 | 0.98 | 0.79 | — | | 19.3 |
| | | opal | | | | | | | | | | 72.2 8.6 |
| 2 wk, | 1 | clear | 3.8 | N/A | N/A | 0.153 | 97.83 | 1.33 | 0.79 | 0.06 | RS007-001 | N/A |
| 40 C. | 2 | clear | 3.1 | | | 0.181 | 97.82 | 1.34 | 0.79 | 0.05 | 33791 | |
| 4 wk, | 1 | clear, sl | 3.5 | N/A | N/A | 0.160 | 98.07 | 1.28 | 0.65 | — | RS007-001 | N/A |
| 40 C. | | opal | | | | | | | | | 33720 | |
| | 2 | clear, sl | 4.2 | | | 0.159 | 98.04 | 1.30 | 0.67 | — | | |
| | | opal | | | | | | | | | | |
| 8 wk, | 1 | opal, | 3.2 | N/A | N/A | 0.168 | 97.86 | 1.48 | 0.65 | — | RS007-001 | N/A |
| 40 C. | | colorless | | | | | | | | | 33693 | |
| | 2 | opal, | 4.9 | | | 0.210 | 97.79 | 1.57 | 0.64 | — | | |
| | | colorless | | | | | | | | | | |
| 12 wk, | 1 | opal, | 5.7 | N/A | 95.5 | 0.230 | 97.19 | 2.43 | 0.26 | 0.11 | RS007-001 | N/A |
| 40 C. | | colorless | | | | | | | | | 33891 | |
| | 2 | opal, | 3.7 | | 96.2 | 0.238 | 97.16 | 2.45 | 0.27 | 0.12 | | |
| | | colorless | | | | | | | | | | |

(v. = very, sl = slightly, opal = opalescent)

TABLE 10
Reconstituted
100 mg/mL Natalizumiab, 100 mg/mL, Sucrose, 15 mM Histidine, 0.05% PS-80
Manufactured:
Testing Start Date: 0.5 mL fill

| Time | | | | | | | | | | | SEC Ref | % Acidic |
|---|---|---|---|---|---|---|---|---|---|---|---|---|
| Point/ | | | Recon Time | KF % | Conc. | Turbidity | % | % | % | % | Std & Avg. | % Main |
| Temp | Vial | Appearance | (min.) | Moisture | (mg/mL) | A360 nm | Monomer | Dimer | Aggregate | LMW | area count | % Basic |
| 0 | 1 | clear, sl opal | 3.8 | 5.9 | 96.0 | 0.095 | 98.10 | 1.04 | 0.79 | 0.08 | RS007-001 | 18.9 |
| | | | | | | | | | | | 3448 | 71.0 |
| | | | | | | | | | | | | 10.1 |
| | 2 | clear, sl opal | 3.3 | | 95.9 | 0.109 | 98.10 | 1.07 | 0.75 | 0.08 | | 19.5 |
| | | | | | | | | | | | | 69.8 |
| | | | | | | | | | | | | 10.6 |
| 4 wk, | 1 | clear, sl opal | 5.8 | N/A | N/A | 0.139 | 98.44 | 0.93 | 0.63 | — | RS007-001 | N/A |
| 5 C. | | | | | | | | | | | 33720 | |
| | 2 | clear, sl opal | 2.2 | | | 0.133 | 98.46 | 0.91 | 0.64 | — | | |
| 8 wk, | 1 | opal, | 7.9 | N/A | N/A | 0.126 | 98.46 | 0.92 | 0.62 | — | RS007-001 | N/A |
| 5 C. | | colorless | | | | | | | | | 33245 | |
| | 2 | opal, | 6.2 | | | 0.114 | 98.46 | 0.92 | 0.63 | — | | |
| | | colorless | | | | | | | | | | |
| 12 wk, | 1 | opal, | 5.9 | N/A | 93.1 | 0.115 | 98.13 | 1.07 | 0.71 | 0.08 | RS007-001 | 19.3 |
| 5 C. | | colorless | | | | | | | | | 33881 | 68.9 |
| | | | | | | | | | | | | 11.8 |
| | 2 | opal, | 2.7 | | 93.1 | 0.138 | 98.01 | 1.24 | 0.67 | 0.09 | | 19.4 |
| | | colorless | | | | | | | | | | 68.7 |
| | | | | | | | | | | | | 11.8 |
| 6 mo, | 1 | opal, | 6.2 | N/A | 93.5 | 0.127 | 98.39 | 1.01 | 0.60 | — | REF025 | 21.3 |
| 5 C. | | colorless | | | | | | | | | (RS007-001) | 66.7 |
| | | | | | | | | | | | 33759 | 12.0 |
| | 2 | opal, | 6.1 | | 93.4 | 0.132 | 98.40 | 1.01 | 0.60 | — | | 21.1 |
| | | colorless | | | | | | | | | | 66.8 |
| | | | | | | | | | | | | 12.0 |
| 12 mo, | 1 | opal, | 7.5 | N/A | 93.3 | 0.125 | 98.34 | 1.00 | 0.67 | — | REF025 | 20.4 |
| 5 C. | | colorless | | | | | | | | | (RS007-001) | 65.5 |
| | | | | | | | | | | | 36185 | 14.1 |
| | 2 | opal, | 8.1 | | 92.8 | 0.126 | 98.33 | 0.99 | 0.68 | — | | 19.7 |
| | | colorless | | | | | | | | | | 66.2 |
| | | | | | | | | | | | | 14.2 |

30° C.

| | | | | | | | | | | | | |
|---|---|---|---|---|---|---|---|---|---|---|---|---|
| 0 | 1 | clear, sl | 3.8 | 5.9 | 96.0 | 0.095 | 98.10 | 1.04 | 0.79 | 0.08 | RS007-001 | 18.9 |
| | | opal | | | | | | | | | 3448 | 71.0 |
| | | | | | | | | | | | | 10.1 |
| | 2 | clear, sl | 3.3 | | 95.9 | 0.109 | 98.10 | 1.07 | 0.75 | 0.08 | | 19.5 |
| | | opal | | | | | | | | | | 69.8 |
| | | | | | | | | | | | | 10.6 |
| 3 wk, | 1 | clear, sl | 5.0 | N/A | N/A | 0.137 | 98.37 | 1.01 | 0.62 | — | RS007-001 | N/A |
| 30 C. | | opal, | | | | | | | | | 33707 | |
| | | colorless | | | | | | | | | | |
| | 2 | clear, sl | 8.4 | | | 0.123 | 98.33 | 1.05 | 0.62 | — | | |
| | | opal | | | | | | | | | | |
| | | colorless | | | | | | | | | | |
| 6 wk, | 1 | sl opal, | 4.6 | N/A | N/A | 0.121 | 98.13 | 1.35 | 0.52 | — | RS007-001 | N/A |
| 30 C. | | colorless | | | | | | | | | 33758 | |
| | 2 | sl opal, | 6.8 | | | 0.123 | 98.14 | 1.36 | 0.50 | — | | |
| | | colorless | | | | | | | | | | |
| 9 wk, | 1 | opal, | 2.3 | N/A | N/A | 0.160 | 98.08 | 1.34 | 0.58 | — | RS007-001 | N/A |
| 30 C. | | colorless | | | | | | | | | 33757 | |
| | 2 | opal, | 4.5 | | | 0.147 | 97.99 | 1.42 | 0.58 | — | | |
| | | colorless | | | | | | | | | | |
| 12 wk, | 1 | opal, | 8.2 | N/A | 89.7 | 0.159 | 96.62 | 3.04 | 0.24 | 0.10 | RS007-001 | 23.5 |
| 30 C. | | colorless | | | | | | | | | 33897 | 66.3 |
| | | | | | | | | | | | | 10.2 |
| | 2 | opal, | 7.5 | | 90.4 | 0.126 | 97.58 | 1.69 | 0.65 | 0.08 | | 22.6 |
| | | colorless | | | | | | | | | | 68.3 |
| | | | | | | | | | | | | 9.1 |
| 6 mo, | 1 | opal, | 7.2 | N/A | 95.7 | 0.188 | 97.45 | 2.14 | 0.25 | 0.16 | REF025 | 22.7 |
| 30 C. | | | | | | | | | | | (RS007-001) | 15.1 |
| | | | | | | | | | | | 33774 | 23.0 |
| | 2 | opal, | 6.2 | | 94.0 | 0.195 | 97.33 | 2.25 | 0.25 | 0.16 | | 62.0 |
| | | colorless | | | | | | | | | | 15.0 |
| 12 mo, | 1 | opal, | 7.6 | N/A | 93.0 | 0.230 | 96.82 | 2.27 | 0.76 | 0.15 | REF025 | 22.9 |
| 30 C. | | slightly | | | | | | | | | (RS007-001) | 58.1 |
| | | yellow | | | | | | | | | 36185 | 19.0 |
| | 2 | opal, | 7.8 | | 93.9 | 0.226 | 96.90 | 2.24 | 0.73 | 0.14 | | 22.9 |
| | | slightly | | | | | | | | | | 58.4 |
| | | yellow | | | | | | | | | | 18.7 |

40° C.

| | | | | | | | | | | | | |
|---|---|---|---|---|---|---|---|---|---|---|---|---|
| 0 | 1 | clear, sl opal | 3.8 | 5.9 | 96.0 | 0.095 | 98.10 | 1.04 | 0.79 | 0.08 | RS007-001 | 18.9 |
| | | | | | | | | | | | 3448 | 71.0 |
| | | | | | | | | | | | | 10.1 |
| | 2 | clear, sl opal | 3.3 | | 95.9 | 0.109 | 98.10 | 1.07 | 0.75 | 0.08 | | 19.5 |
| | | | | | | | | | | | | 69.8 |
| | | | | | | | | | | | | 10.6 |
| 2 wk, | 1 | clear | 5.4 | N/A | N/A | 0.142 | 96.96 | 2.77 | 0.20 | 0.07 | RS007-001 | N/A |
| 40 C. | 2 | clear | 7.3 | | | 0.149 | 97.53 | 1.73 | 0.69 | 0.06 | 33791 | |
| 4 wk, | 1 | clear, sl opal | 6.8 | N/A | N/A | 0.189 | 97.14 | 1.95 | 0.70 | 0.2 | RS007-001 | N/A |
| 40 C. | 2 | clear, sl opal | 5.3 | | | 0.195 | 97.33 | 1.82 | 0.67 | 0.18 | 33720 | |
| 8 wk, | 1 | opal, | 6.5 | N/A | N/A | 0.227 | 96.94 | 2.76 | 0.11 | 0.20 | RS007-001 | N/A |
| 40 C. | | colorless | | | | | | | | | 33693 | |
| | 2 | opal, | 6.0 | | | 0.226 | 97.07 | 2.16 | 0.57 | 0.20 | | |
| | | colorless | | | | | | | | | | |
| 12 wk, | 1 | opal, | 3.9 | N/A | 94.2 | 0.161 | 96.80 | 2.87 | 0.23 | 0.10 | RS007-001 | 21.0 |
| 40 C. | | colorless | | | | | | | | | 33891 | 55.1 |
| | | | | | | | | | | | | 24.0 |
| | 2 | opal, | 4.1 | | 91.9 | 0.173 | 96.96 | 2.69 | 0.24 | 0.10 | | 21.8 |
| | | colorless | | | | | | | | | | 55.8 |
| | | | | | | | | | | | | 22.4 |

(v. = very, sl = slightly, opal = opalescent)

TABLE 11
Reconstituted
200 mg/mL Natalizumiab, 100 mg/mL Sucrose, 24 mM Histidine, 0.08% PS-80
Manufactured:
Testing Start Date: 0.5 mL fill

| Time | | | Recon | | | | | | | | SEC Ref | % Acidic |
|---|---|---|---|---|---|---|---|---|---|---|---|---|
| Point/ | | | Time | KF % | Conc. | Turbidity | % | % | % | | Std & Avg. | % Main |
| Temp | Vial | Appearance | (min.) | Moisture | (mg/mL) | A360 nm | Monomer | Dimer | Aggregate | % LMW | area count | % Basic |
| 0 | 1 | clear, sl opal | 9.5 | 5.8 | 172.0 | 0.095 | 97.96 | 1.10 | 0.86 | 0.08 | RS007-001 | 19.7 |
| | | | | | | | | | | | 3448 | 71.8 8.5 |
| | 2 | clear, sl opal | 10.5 | | 173.2 | 0.106 | 97.96 | 1.10 | 0.86 | 0.08 | | 19.1 70.9 |
| | | | | | | | | | | | | 10.0 |
| 4 wk, | 1 | clear, sl opal | 4.8 | N/A | N/A | 0.157 | 97.91 | 1.16 | 0.79 | 0.13 | RS007-001 | N/A |
| 5 C. | 2 | clear, sl opal | 5.8 | | | 0.153 | 97.89 | 1.18 | 0.80 | 0.13 | 33720 | |
| 8 wk, | 1 | opal, colorless | 6.5 | N/A | N/A | 0.141 | 98.18 | 1.10 | 0.72 | — | RS007-001 | N/A |
| 5 C. | 2 | opal, colorless | 7.5 | | | 0.152 | 98.21 | 1.13 | 0.66 | — | 33245 | |
| 12 wk, | 1 | opal, colorless | 6.0 | N/A | 154.5 | 0.154 | 97.90 | 1.25 | 0.76 | 0.08 | RS007-001 | 18.6 |
| 5 C. | | | | | | | | | | | 33881 | 63.8 |
| | | | | | | | | | | | | 17.7 |
| | 2 | opal, colorless | 5.6 | | 170.6 | 0.150 | 97.88 | 1.27 | 0.77 | 0.09 | | 19.9 |
| | | | | | | | | | | | | 68.9 |
| | | | | | | | | | | | | 11.1 |
| 6 mo, | 1 | opal, colorless | 7.3 | N/A | 174.9 | 0.167 | 98.07 | 1.28 | 0.66 | — | REF025 | 21.2 |
| 5 C. | | | | | | | | | | | (RS007-001) | 66.4 |
| | | | | | | | | | | | 33759 | 12.4 |
| | 2 | opal, colorless | 6.6 | | 175.3 | 0.167 | 98.08 | 1.26 | 0.66 | — | | 21.0 |
| | | | | | | | | | | | | 66.6 |
| | | | | | | | | | | | | 12.4 |
| 12 mo, | 1 | opal, colorless | 6.7 | N/A | 178.1 | 0.156 | 97.86 | 1.40 | 0.73 | — | REF025 | 20.0 |
| 5 C. | | | | | | | | | | | (RS007-001) | 65.5 |
| | | | | | | | | | | | 36185 | 14.5 |
| | 2 | opal, colorless | 3.8 | | 172.2 | 0.152 | 97.93 | 1.33 | 0.74 | — | | 19.4 |
| | | | | | | | | | | | | 65.6 |
| | | | | | | | | | | | | 14.9 |

30° C.

| | | | | | | | | | | | | |
|---|---|---|---|---|---|---|---|---|---|---|---|---|
| 0 | 1 | clear, sl opal | 9.5 | 5.8 | 172.0 | 0.095 | 97.96 | 1.10 | 0.86 | 0.08 | RS007-001 | 19.7 |
| | | | | | | | | | | | 3448 | 71.8 |
| | | | | | | | | | | | | 8.5 |
| | 2 | clear, sl opal | 10.5 | | 173.2 | 0.106 | 97.96 | 1.10 | 0.86 | 0.08 | | 19.1 |
| | | | | | | | | | | | | 70.9 |
| | | | | | | | | | | | | 10.0 |
| 3 wk, | 1 | clear, sl opal, | 4.2 | N/A | N/A | 0.165 | 97.79 | 1.48 | 0.73 | — | RS007-001 | N/A |
| 30 C. | | colorless | | | | | | | | | 33707 | |
| | 2 | clear, sl opal, | 4.3 | | | 0.169 | 97.77 | 1.50 | 0.73 | — | | |
| | | colorless | | | | | | | | | | |
| 6 wk, | 1 | v. sl opal, | 7.1 | N/A | N/A | 0.157 | 97.74 | 1.68 | 0.58 | — | RS007-001 | N/A |
| 30 C. | | colorless | | | | | | | | | 33758 | |
| | 2 | sl opal, | 5.9 | | | 0.163 | 97.77 | 1.65 | 0.59 | — | | |
| | | colorless | | | | | | | | | | |
| 9 wk, | 1 | opal, | 4.0 | N/A | N/A | 0.186 | 97.52 | 1.78 | 0.70 | — | RS007-001 | N/A |
| 30 C. | | colorless | | | | | | | | | 33757 | |
| | 2 | opal, | 3.8 | | | 0.185 | 97.41 | 1.89 | 0.70 | — | | |
| | | colorless | | | | | | | | | | |
| 12 wk, | 1 | opal, | 4.9 | N/A | 174.5 | 0.178 | 96.88 | 2.23 | 0.80 | 0.09 | RS007-001 | 20.0 |
| 30 C. | | colorless | | | | | | | | | 33897 | 59.0 |
| | | | | | | | | | | | | 21.0 |
| | 2 | opal, | 5.4 | | 177.6 | 0.183 | 96.93 | 2.20 | 0.79 | 0.08 | | 19.9 |
| | | colorless | | | | | | | | | | 58.1 |
| | | | | | | | | | | | | 22.0 |
| 6 mo, | 1 | opal, | 6.0 | N/A | 173.2 | 0.237 | 96.16 | 3.45 | 0.39 | — | REF025 | 21.8 |
| 30 C. | | colorless | | | | | | | | | (RS007-001) | 63.3 |
| | | | | | | | | | | | 33774 | 15.0 |
| | 2 | opal, | 4.9 | | 174.1 | 0.227 | 96.13 | 3.47 | 0.40 | — | | 21.8 |
| | | colorless | | | | | | | | | | 63.3 |
| | | | | | | | | | | | | 15.0 |
| 12 mo, | 1 | opal, slightly | 7.0 | N/A | 171.2 | 0.221 | 95.35 | 3.56 | 1.09 | — | REF025 | 20.9 |
| 30 C. | | yellow | | | | | | | | | (RS007-001) | 61.1 |
| | | | | | | | | | | | 36185 | 17.9 |
| | 2 | opal, slightly | 6.5 | | 168.8 | 0.220 | 95.26 | 3.60 | 1.14 | — | | 20.8 |
| | | yellow | | | | | | | | | | 61.2 |
| | | | | | | | | | | | | 18.0 |

40° C.

| | | | | | | | | | | | | |
|---|---|---|---|---|---|---|---|---|---|---|---|---|
| 0 | 1 | clear, sl opal | 9.5 | 5.8 | 172.0 | 0.095 | 97.96 | 1.10 | 0.86 | 0.08 | RS007-001 | 19.7 |
| | | | | | | | | | | | 3448 | 71.8 |
| | | | | | | | | | | | | 8.5 |
| | 2 | clear, sl opal | 10.5 | | 173.2 | 0.106 | 97.96 | 1.10 | 0.86 | 0.08 | | 19.1 |
| | | | | | | | | | | | | 70.9 |
| | | | | | | | | | | | | 10.0 |
| 2 wk, | 1 | clear | 8.3 | N/A | N/A | 0.180 | 96.93 | 2.17 | 0.84 | 0.06 | RS007-001 | N/A |
| 40 C. | 2 | clear | 3.9 | | | 0.166 | 96.89 | 2.20 | 0.85 | 0.07 | 33791 | |
| 4 wk, | 1 | clear, sl opal | 3.6 | N/A | N/A | 0.196 | 96.37 | 2.62 | 0.88 | 0.14 | RS007-001 | N/A |
| 40 C. | 2 | clear, sl opal | 2.4 | | | 0.203 | 96.34 | 2.62 | 0.89 | 0.15 | 33720 | |
| 8 wk, | 1 | opal, colorless | 4.6 | N/A | N/A | 0.214 | 95.68 | 3.27 | 0.93 | 0.12 | RS007-001 | N/A |
| 40 C. | 2 | opal, colorless | 6.2 | | | 0.208 | 95.98 | 3.18 | 0.84 | — | 33693 | |
| 12 wk, | 1 | opal, colorless | 7.6 | N/A | 167.6 | 0.229 | 94.92 | 3.94 | 1.05 | 0.10 | RS007-001 | 19.9 |
| 40 C. | | | | | | | | | | | 33891 | 53.5 |
| | | | | | | | | | | | | 26.6 |
| | 2 | opal, colorless | 4.8 | | 167.0 | 0.241 | 94.90 | 3.94 | 1.06 | 0.09 | | 20.5 |
| | | | | | | | | | | | | 54.6 |
| | | | | | | | | | | | | 24.9 |

TABLE 12
Reconstituted
225 mg/mL Natalizuniab, 120 mg/mL Sucrose, 18 mM Histidine, 0.06% PS-80
Manufactured:
Testing Start Date: 0.5 mL fill

| Time | | | | | | | | | | | SEC Ref | % Acidic |
|---|---|---|---|---|---|---|---|---|---|---|---|---|
| Point/ | | | Recon Time | KF % | Conc. | Turbidity | % | % | % | % | Std & Avg. | % Main |
| Temp | Vial | Appearance | (min.) | Moisture | (mg/mL) | A360 nm | Monomer | Dimer | Aggregate | LMW | area count | % Basic |
| 0 | 1 | clear, sl opal | 6.2 | 19.0 | 194.7 | 0.093 | 96.90 | 1.63 | 1.39 | 0.08 | RS007-001 | 19.7 |
| | | | | | | | | | | | 3448 | 70.3 |
| | | | | | | | | | | | | 10.0 |
| | 2 | clear, sl opal | | 20.5 | 184.1 | 0.097 | 96.88 | 1.64 | 1.39 | 0.09 | | 17.8 |
| | | | | | | | | | | | | 73.3 |
| | | | | | | | | | | | | 8.9 |
| 4 wk, | 1 | clear, sl opal | 14.3 | N/A | N/A | 0.136 | 96.82 | 1.72 | 1.32 | 0.14 | RS007-001 | N/A |
| 5 C. | 2 | clear, sl opal | 18.0 | | | 0.143 | 96.87 | 1.67 | 1.33 | 0.13 | 33720 | |
| 8 wk, | 1 | opal, | 16.3 | N/A | N/A | 0.143 | 96.94 | 1.69 | 1.25 | 0.12 | RS007-001 | N/A |
| 5 C. | | colorless | | | | | | | | | 33245 | |
| | 2 | opal, | 18.8 | | | 0.142 | 96.91 | 1.69 | 1.28 | 0.11 | | |
| | | colorless | | | | | | | | | | |
| 12 wk, | 1 | opal, | 15.3 | N/A | 224.7 | 0.145 | 96.82 | 1.85 | 1.23 | 0.10 | RS007-001 | |
| 5 C. | | colorless | | | | | | | | | 33881 | |
| | 2 | opal, | 27.0 | | 222.3 | 0.134 | 96.88 | 1.80 | 1.24 | 0.08 | | |
| | | colorless | | | | | | | | | | |
| 6 mo, | 1 | opal, | 15.5 | N/A | 196.9 | 0.149 | 97.10 | 1.81 | 1.09 | — | REF025 | 20.9 |
| 5 C. | | colorless | | | | | | | | | (RS007-001) | 66.6 |
| | | | | | | | | | | | 33759 | 12.5 |
| | 2 | opal, | 17.1 | | 197.1 | 0.152 | 97.13 | 17.9 | 1.08 | — | | 20.8 |
| | | colorless | | | | | | | | | | 66.7 |
| | | | | | | | | | | | | 12.5 |

30° C.

| | | | | | | | | | | | | |
|---|---|---|---|---|---|---|---|---|---|---|---|---|
| 0 | 1 | clear, sl opal | 19.0 | 6.2 | 194.7 | 0.093 | 96.90 | 1.63 | 1.39 | 0.08 | RS007-001 | 19.7 |
| | | | | | | | | | | | 3448 | 70.3 |
| | | | | | | | | | | | | 10.0 |
| | 2 | clear, sl opal | 20.5 | | 184.1 | 0.097 | 96.88 | 1.64 | 1.39 | 0.09 | | 17.8 |
| | | | | | | | | | | | | 73.3 |
| | | | | | | | | | | | | 8.9 |
| 3 wk, | 1 | clear, sl | 8.3 | N/A | N/A | 0.173 | 96.48 | 2.12 | 1.29 | — | RS007-001 | N/A |
| 30 C. | | opal, | | | | | | | | | 33707 | |
| | | colorless | | | | | | | | | | |
| | 2 | clear, sl | 12.9 | | | 0.168 | 96.49 | 2.11 | 1.28 | — | | |
| | | opal, | | | | | | | | | | |
| | | colorless | | | | | | | | | | |
| 6 wk, | 1 | sl opal, | 11.9 | N/A | N/A | 0.143 | 96.41 | 2.28 | 1.16 | 0.14 | RS007-001 | N/A |
| 30 C. | | colorless | | | | | | | | | 33758 | |
| | 2 | sl opal, | 27.6 | | | 0.166 | 96.04 | 2.67 | 1.15 | 0.14 | | |
| | | colorless | | | | | | | | | | |
| 9 wk, | 1 | opal, | 21.6 | N/A | N/A | 0.178 | 95.91 | 2.84 | 1.25 | — | RS007-001 | N/A |
| 30 C. | | colorless | | | | | | | | | 33757 | |
| | 2 | opal, | 22.4 | | | 0.170 | 96.39 | 2.40 | 1.21 | — | | |
| | | colorless | | | | | | | | | | |
| 12 wk, | 1 | opal, | 19.3 | N/A | 211.6 | 0.174 | 95.81 | 2.83 | 1.26 | 0.09 | RS007-001 | N/A |
| 30 C. | | colorless | | | | | | | | | 33897 | |
| | 2 | opal, | 16.8 | | 223.5 | 0.173 | 95.86 | 2.80 | 1.26 | 0.09 | | |
| | | colorless | | | | | | | | | | |
| 6 mo, | 1 | opal, | 24.4 | N/A | 181.3 | 0.238 | 95.10 | 3.57 | 1.33 | — | REF025 | 21.6 |
| 30 C. | | colorless | | | | | | | | | (RS007-001) | 63.3 |
| | | | | | | | | | | | 33774 | 15.2 |
| | 2 | opal, | 22.9 | | 183.7 | 0.225 | 95.16 | 3.57 | 1.27 | — | | 21.6 |
| | | colorless | | | | | | | | | | 63.4 |
| | | | | | | | | | | | | 15.0 |

40° C.

| | | | | | | | | | | | | |
|---|---|---|---|---|---|---|---|---|---|---|---|---|
| 0 | 1 | clear, sl opal | 19.0 | 6.2 | 194.7 | 0.093 | 96.90 | 1.63 | 1.39 | 0.08 | RS007-001 | 19.7 |
| | | | | | | | | | | | 3448 | 70.3 |
| | | | | | | | | | | | | 10.0 |
| | 2 | clear, sl opal | 20.5 | | 184.1 | 0.097 | 96.88 | 1.64 | 1.39 | 0.09 | | 17.8 |
| | | | | | | | | | | | | 73.3 |
| | | | | | | | | | | | | 8.9 |
| 2 wk, | 1 | clear | 14.5 | N/A | N/A | 0.161 | 95.17 | 3.40 | 1.36 | 0.07 | RS007-001 | N/A |
| 40 C. | 2 | clear | 15.0 | | | 0.153 | 95.87 | 2.74 | 1.33 | 0.06 | 33791 | |
| 4 wk, | 1 | clear, sl opal | 13.4 | N/A | N/A | 0.180 | 95.26 | 3.19 | 1.39 | 0.16 | RS007-001 | N/A |
| 40 C. | 2 | clear, sl opal | 19.0 | | | 0.179 | 95.23 | 3.22 | 1.40 | 0.15 | 33720 | |
| 8 wk, | 1 | opal, | 18.3 | N/A | N/A | 0.210 | 94.86 | 3.77 | 1.37 | — | RS007-001 | N/A |
| 40 C. | | colorless | | | | | | | | | 33693 | |
| | 2 | opal, | 15.9 | | | 0.211 | 94.10 | 4.34 | 1.41 | 0.15 | | |
| | | colorless | | | | | | | | | | |
| 12 wk, | 1 | opal, | 20.8 | N/A | 194.2 | 0.248 | 93.87 | 4.54 | 1.52 | 0.07 | RS007-001 | N/A |
| 40 C. | | colorless | | | | | | | | | 33891 | |
| | 2 | opal, | 16.4 | | 215.4 | 0.235 | 94.12 | 4.31 | 1.47 | 0.10 | | |
| | | colorless | | | | | | | | | | |

TABLE 13
Pre-Lyo
50 mg/mL Natalizumab, 25 mg/mL Sucrose, 6 mM Histidine, 15 mM NaCl,
0.02% PS-80
Prepared:
Testing Start Date: 0.5 mL fill

| Time | | | | | | | | | | % Acidic |
|---|---|---|---|---|---|---|---|---|---|---|
| Point/ | | Appearance, | Conc. | Turbidity | % | % | % | % | SEC Ref Std & | % Main |
| Temp | Vial | pH | (mg/mL) | A360 nm | Monomer | Dimer | Aggregate | LMW | Avg. area count | % Basic |
| 0 | 1 | clear, sl | 50.0 | 0.181 | 98.31 | 1.04 | 0.57 | 0.07 | RS007-001 | 20.8 |
| | | opal, | 50.7 | | | | | | 33834 | 68.0 |
| | | colorless | | | | | | | | 11.3 |
| 4 wk, | 1 | sl opal, | N/A | 0.105 | 98.31 | 1.04 | 0.54 | 0.11 | RS007-001 | N/A |
| 5 C. | | colorless | | | | | | | 33626 | |
| | 2 | sl opal, | | 0.093 | 98.31 | 1.04 | 0.54 | 0.12 | | |
| | | colorless | | | | | | | | |
| 8 wk, | 1 | sl opal, | 50.0 | 0.103 | 98.59 | 0.96 | 0.45 | — | RS007-001 | N/A |
| 5 C. | | colorless | | | | | | | 33670 | |
| | 2 | sl opal, | 50.0 | 0.098 | 98.60 | 0.96 | 0.44 | — | | |
| | | colorless | | | | | | | | |
| 12 wk, | 1 | sl opal, | N/A | 0.098 | 98.36 | 1.07 | 0.47 | 0.10 | REF025 | N/A |
| 5 C. | | colorless | | | | | | | (RS007-001) | |
| | 2 | sl opal, | | 0.107 | 98.35 | 1.07 | 0.47 | 0.11 | 33608 | |
| | | colorless | | | | | | | | |

30° C.

| Time | | | | | | | | | | % Acidic |
|---|---|---|---|---|---|---|---|---|---|---|
| Point/ | | | Conc. | Turbidity | % | % | % | % | SEC Ref Std & | % Main |
| Temp | Vial | Appearance | (mg/mL) | A360 nm | Monomer | Dimer | Aggregate | LMW | Avg. area count | % Basic |
| 0 | 1 | clear, sl | 50.0 | 0.181 | 98.31 | 1.04 | 0.57 | 0.07 | RS007-001 | 20.8 |
| | | opal, | 50.7 | | | | | | 33834 | 68.0 |
| | | colorless | | | | | | | | 11.3 |
| 3 wk, | 1 | sl opal, | N/A | 0.038 | 98.54 | 0.95 | 0.15 | 0.06 | RS007-001 | N/A |
| 30 C. | | colorless | | | | | | | 33758 | |
| | 2 | sl opal, | | 0.034 | 98.38 | 0.99 | 0.45 | 0.14 | | |
| | | colorless | | | | | | | | |
| 6 wk, | 1 | sl opal, | N/A | 0.109 | 98.54 | 0.95 | 0.41 | 0.10 | RS007-001 | N/A |
| 30 C. | | colorless | | | | | | | 33506 | |
| | 2 | sl opal, | | 0.108 | 98.53 | 0.95 | 0.42 | 0.10 | | |
| | | colorless | | | | | | | | |
| 9 wk, | 1 | sl opal, | N/A | 0.104 | 98.50 | 0.99 | 0.35 | 0.16 | RS007-001 | N/A |
| 30 C. | | colorless | | | | | | | 33775 | |
| | 2 | sl opal, | | 0.102 | 98.50 | 0.99 | 0.35 | 0.16 | | |
| | | colorless | | | | | | | | |
| 12 wk, | 1 | sl opal, | N/A | 0.102 | 98.04 | 1.12 | 0.37 | 0.47 | REF025 | N/A |
| 30 C. | | colorless | | | | | | | (RS007-001) | |
| | 2 | sl opal, | | 0.106 | 98.05 | 1.10 | 0.38 | 0.47 | 33586 | |
| | | colorless | | | | | | | | |

40° C.

| Time | | | | | | | | | | % Acidic |
|---|---|---|---|---|---|---|---|---|---|---|
| Point/ | | | Conc. | Turbidity | % | % | % | % | SEC Ref Std & | % Main |
| Temp | Vial | Appearance | (mg/mL) | A360 nm | Monomer | Dimer | Aggregate | LMW | Avg. area count | % Basic |
| 0 | 1 | clear, sl | 50.0 | 0.181 | 98.31 | 1.04 | 0.57 | 0.07 | RS007-001 | 20.8 |
| | | opal, | 50.7 | | | | | | 33834 | 68.0 |
| | | colorless | | | | | | | | 11.3 |
| 2 wk, | 1 | clear, sl | N/A | 0.105 | 98.05 | 1.01 | 0.46 | 0.48 | RS007-001 | N/A |
| 40 C. | | opal, | | | | | | | 33732 | |
| | | colorless | | | | | | | | |
| | 2 | clear, sl | | 0.109 | 98.05 | 1.01 | 0.46 | 0.48 | | |
| | | opal, | | | | | | | | |
| | | colorless | | | | | | | | |
| 4 wk, | 1 | sl opal, | N/A | 0.147 | 95.23 | 1.08 | 0.43 | 3.25 | RS007-001 | N/A |
| 40 C. | | colorless | | | | | | | 34124 | |
| | 2 | sl opal, | | 0.106 | 95.20 | 1.08 | 0.42 | 3.30 | | |
| | | colorless | | | | | | | | |
| 8 wk, | 1 | sl opal, | 52.0 | 0.121 | 89.74 | 1.34 | 0.62 | 8.29 | RS007-001 | N/A |
| 40 C. | | colorless | | | | | | | 33852 | |
| | 2 | sl opal, | 54.0 | 0.116 | 89.53 | 1.22 | 0.65 | 8.59 | | |
| | | colorless | | | | | | | | |
| 12 wk, | 1 | sl opal, | N/A | 0.128 | 86.74 | 1.47 | 1.39 | 10.41 | REF025 | N/A |
| 40 C. | | colorless | | | | | | | (RS007-001) | |
| | 2 | sl opal, | | 0.131 | 86.46 | 1.47 | 1.35 | 10.72 | 33590 | |
| | | colorless | | | | | | | | |

TABLE 14
Pre-Lyo
50 mg/mL Natalizumab, 25 mg/mL Sucrose, 6 mM Histidine, 0.02% PS-20
Prepared:
Testing Start Date: 0.5 mL fill

| Time | | | | | | | | | | % Acidic |
|---|---|---|---|---|---|---|---|---|---|---|
| Point/ | | Appearance, | Conc. | Turbidity | % | % | % | % | SEC Ref Std & | % Main |
| Temp | Vial | pH | (mg/mL) | A360 nm | Monomer | Dimer | Aggregate | LMW | Avg. area count | % Basic |
| 0 | 1 | clear, sl | 53.7 | 0.050 | 98.22 | 1.06 | 0.65 | 0.07 | RS007-001 | 21.0 |
| | | opal, | 51.7 | | | | | | 33834 | 67.9 |
| | | colorless, 6.11 | | | | | | | | 11.1 |
| 4 wk, | 1 | v. sl opal, | N/A | 0.058 | 98.38 | 1.00 | 0.59 | 0.03 | RS007-001 | N/A |
| 5 C. | | colorless | | | | | | | 33626 | |
| | 2 | v. sl opal, | | 0.065 | 98.39 | 0.99 | 0.58 | 0.03 | | |
| | | colorless | | | | | | | | |
| 8 wk, | 1 | sl opal, | 50.1 | 0.060 | 98.59 | 0.95 | 0.46 | — | RS007-001 | N/A |
| 5 C. | | colorless | | | | | | | 33670 | |
| | 2 | sl opal, | 49.8 | 0.068 | 98.59 | 0.96 | 0.45 | — | | |
| | | colorless | | | | | | | | |
| 12 wk, | 1 | sl opal, | N/A | 0.066 | 98.31 | 1.08 | 0.50 | 0.12 | REF025 | N/A |
| 5 C. | | colorless | | | | | | | (RS007-001) | |
| | 2 | sl opal, | | 0.065 | 98.31 | 1.08 | 0.49 | 0.12 | 33608 | |
| | | colorless | | | | | | | | |

30° C.

| Time | | | | | | | | | | % Acidic |
|---|---|---|---|---|---|---|---|---|---|---|
| Point/ | | | Conc. | Turbidity | % | % | % | % | SEC Ref Std & | % Main |
| Temp | Vial | Appearance, | (mg/mL) | A360 nm | Monomer | Dimer | Aggregate | LMW | Avg. area count | % Basic |
| 0 | 1 | clear, sl opal, | 53.7 | 0.050 | 98.22 | 1.06 | 0.65 | 0.07 | RS007-001 | 21.0 |
| | | colorless, 6.11 | 51.7 | | | | | | 33834 | 67.9 |
| | | | | | | | | | | 11.1 |
| 3 wk, | 1 | v. sl opal, | N/A | 0.029 | 98.31 | 1.01 | 0.49 | 0.16 | RS007-001 | N/A |
| 30 C. | | colorless | | | | | | | 33758 | |
| | 2 | v. sl opal, | N/A | 0.019 | 98.31 | 1.02 | 0.52 | 0.16 | | |
| | | colorless | | | | | | | | |
| 6 wk, | 1 | sl opal, | N/A | 0.063 | 98.51 | 0.95 | 0.44 | 0.10 | RS007-001 | N/A |
| 30 C. | | colorless | | | | | | | 33506 | |
| | 2 | sl opal, | | 0.061 | 98.33 | 1.00 | 0.47 | 0.21 | | |
| | | colorless | | | | | | | | |
| 9 wk, | 1 | sl opal, | N/A | 0.054 | 98.24 | 1.05 | 0.39 | 0.32 | RS007-001 | N/A |
| 30 C. | | colorless | | | | | | | 33775 | |
| | 2 | sl opal, | | 0.046 | 98.25 | 1.05 | 0.39 | 0.32 | | |
| | | colorless | | | | | | | | |
| 12 wk, | 1 | sl opal, | N/A | 0.061 | 98.14 | 1.07 | 0.39 | 0.40 | REF025 | N/A |
| 30 C. | | colorless | | | | | | | (RS007-001) | |
| | 2 | sl opal, | | 0.060 | 98.13 | 1.07 | 0.39 | 0.41 | 33586 | |
| | | colorless | | | | | | | | |

40° C.

| Time | | | | | | | | | | % Acidic |
|---|---|---|---|---|---|---|---|---|---|---|
| Point/ | | | Conc. | Turbidity | % | % | % | % | SEC Ref Std & | % Main |
| Temp | Vial | Appearance | (mg/mL) | A360 nm | Monomer | Dimer | Aggregate | LMW | Avg. area count | % Basic |
| 0 | 1 | clear, sl opal, | 53.7 | 0.050 | 98.22 | 1.06 | 0.65 | 0.07 | RS007-001 | 21.0 |
| | | colorless, 6.11 | 51.7 | | | | | | 33834 | 67.9 |
| | | | | | | | | | | 11.1 |
| 2 wk, | 1 | v. sl opal, | N/A | 0.102 | 98.06 | 1.03 | 0.48 | 0.43 | RS007-001 | N/A |
| 30 C. | | colorless | | | | | | | 33732 | |
| | 2 | v. sl opal, | N/A | 0.098 | 98.08 | 1.02 | 0.47 | 0.43 | | |
| | | colorless | | | | | | | | |
| 4 wk, | 1 | sl opal, | N/A | 0.099 | 95.41 | 1.09 | 0.47 | 0.10 | RS007-001 | N/A |
| 40 C. | | colorless | | | | | | | 34124 | |
| | 2 | sl opal, | | 0.081 | 95.25 | 1.11 | 0.45 | 3.18 | | |
| | | colorless | | | | | | | | |
| 8 wk, | 1 | sl opal, | 53.5 | 0.104 | 90.07 | 1.37 | 0.80 | 7.76 | RS007-001 | N/A |
| 40 C. | | colorless | | | | | | | 33852 | |
| | 2 | sl opal, | 53.9 | 0.097 | 89.83 | 1.29 | 0.73 | 8.15 | | |
| | | colorless | | | | | | | | |
| 12 wk, | 1 | sl opal, | N/A | 0.101 | 89.27 | 0.82 | 1.02 | 8.89 | REF025 | N/A |
| 40 C. | | colorless | | | | | | | (RS007-001) | |
| | 2 | sl opal, | | 0.097 | 89.09 | 0.80 | 0.98 | 9.12 | 33590 | |
| | | colorless | | | | | | | | |

TABLE 15
Pre-Lyo
50 mg/mL Natalizumab, 25 mg/mL Trehalose, 6 mM Histidine, 0.02% PS-80
Prepared:
Testing Start Date: 0.5 mL fill

| Time | | | | | | | | | | % Acidic |
|---|---|---|---|---|---|---|---|---|---|---|
| Point/ | | Appearance, | Conc. | Turbidity | % | % | % | % | SEC Ref Std & | % Main |
| Temp | Vial | pH | (mg/mL) | A360 nm | Monomer | Dimer | Aggregate | LMW | Avg. area count | % Basic |
| 0 | 1 | clear, sl opal, | 51.0 | 0.035 | 98.19 | 1.06 | 0.68 | 0.07 | RS007-001 | 19.7 |
| | | colorless, | 51.5 | | | | | | 33834 | 69.3 |
| | | 6.13 | | | | | | | | 11.0 |
| 4 wk, | 1 | v. sl opal, | N/A | 0.056 | 98.26 | 1.05 | 0.60 | 0.10 | RS007-001 | N/A |
| 5 C. | | colorless | | | | | | | 33626 | |
| | 2 | v. sl opal, | | 0.050 | 98.44 | 0.99 | 0.53 | 0.03 | | |
| | | colorless | | | | | | | | |
| 8 wk, | 1 | sl opal, | 52.7 | 0.076 | 98.59 | 0.96 | 0.45 | — | RS007-001 | N/A |
| 5 C. | | colorless | | | | | | | 33670 | |
| | 2 | sl opal, | 50.1 | 0.093 | 98.58 | 0.97 | 0.46 | — | | |
| | | colorless | | | | | | | | |
| 12 wk, | 1 | sl opal, | N/A | 0.063 | 98.35 | 1.08 | 0.48 | 0.10 | REF025 | N/A |
| 5 C. | | colorless | | | | | | | (RS007-001) | |
| | 2 | sl opal, | | 0.057 | 98.34 | 1.08 | 0.48 | 0.11 | 33608 | |
| | | colorless | | | | | | | | |

30° C.

| Time | | | | | | | | | | % Acidic |
|---|---|---|---|---|---|---|---|---|---|---|
| Point/ | | | Conc. | Turbidity | % | % | % | % | SEC. Ref Std & | % Main |
| Temp. | Vial | Appearance | (mg/mL) | A360 nm | Monomer | Dimer | Aggregate | LMW | Avg. area count | % Basic |
| 0 | 1 | clear, sl opal, | 51.0 | 0.035 | 98.19 | 1.06 | 0.68 | 0.07 | RS007-001 | 19.7 |
| | | colorless, | 51.5 | | | | | | 33834 | 69.3 |
| | | 6.13 | | | | | | | | 11.0 |
| 3 wk, | 1 | sl opal, | N/A | 0.006 | 98.33 | 1.00 | 0.51 | 0.15 | RS007-001 | N/A |
| 30 C. | | colorless | | | | | | | 33758 | |
| | 2 | sl opal, | | 0.010 | 98.33 | 1.01 | 0.51 | 0.15 | | |
| | | colorless | | | | | | | | |
| 6 wk, | 1 | sl opal, | N/A | 0.061 | 98.32 | 1.00 | 0.47 | 0.21 | RS007-001 | N/A |
| 30 C. | | colorless | | | | | | | 33506 | |
| | 2 | sl opal, | N/A | 0.060 | 98.51 | 0.95 | 0.44 | 0.11 | | |
| | | colorless | | | | | | | | |
| 9 wk, | 1 | sl opal, | N/A | 0.059 | 98.21 | 1.08 | 0.38 | 0.32 | RS007-001 | N/A |
| 30 C. | | colorless | | | | | | | 33775 | |
| | | sl opal, | 0.056 | 98.23 | 1.05 | 0.40 | 0.32 | | | |
| | | colorless | | | | | | | | |
| 12 wk, | 2 | sl opal, | | 0.069 | 98.12 | 1.08 | 0.39 | 0.41 | REF025 | N/A |
| 30 C. | | colorless | | | | | | | (RS007-001) | N/A |
| | | sl opal, | | 0.067 | 98.06 | 1.15 | 0.38 | 0.42 | 33586 | |
| | | colorless | | | | | | | | |

40° C.

| Time | | | | | | | | | | % Acidic |
|---|---|---|---|---|---|---|---|---|---|---|
| Point/ | | | Conc. | Turbidity | % | % | % | % | SEC. Ref Std & | % Main |
| Temp. | Vial | Appearance | (mg/mL) | A360 nm | Monomer | Dimer | Aggregate | LMW | Avg. area count | % Basic |
| 0 | 1 | clear, sl opal, | 51.0 | 0.035 | 98.19 | 1.06 | 0.68 | 0.07 | RS0007-001 | 19.7 |
| | | colorless, | 51.5 | | | | | | 33834 | 69.3 |
| | | 6.13 | | | | | | | | 11.0 |
| 2 wk, | 1 | clear, sl opal, | N/A | 0.065 | 98.07 | 1.03 | 0.48 | 0.42 | RS0007-001 | N/A |
| 40 C. | | colorless | | | | | | | 33732 | |
| | 2 | clear, sl opal, | | 0.069 | 98.08 | 1.02 | 0.48 | 0.42 | | |
| | | colorless | | | | | | | | |
| 4 wk, | 1 | sl opal, | N/A | 0.061 | 95.49 | 1.09 | 0.48 | 2.94 | RS0007-001 | N/A |
| 40 C. | | colorless | | | | | | | 34124 | |
| | 2 | v. sl opal, | | 0.060 | 95.37 | 1.09 | 0.46 | 3.08 | | |
| | | colorless | | | | | | | | |
| 8 wk, | 1 | sl opal, | 52.9 | 0.085 | 89.95 | 1.32 | 0.82 | 7.91 | RS0007-001 | N/A |
| 40 C. | | colorless | (potency = | | | | | | 33852 | |
| | 2 | sl opal, | 78.1%) | 0.081 | 89.89 | 1.27 | 0.81 | 8.04 | | |
| | | colorless | | | | | | | | |
| 12 wk, | 1 | sl opal, | 53.4 | 0.099 | 87.14 | 1.56 | 1.51 | 9.78 | REF025 | N/A |
| 40 C. | | colorless | | | | | | | (RS007-001) | |
| | 2 | sl opal, | N/A | 0.110 | 86.16 | 1.63 | 1.49 | 10.72 | 33590 | |
| | | colorless | | | | | | | | |

TABLE 16
Pre-Lyo
40 mg/mL Natalizumab, 25 mg/mL Sucrose, 6 mM Histidin, 0.02% PS-80
Prepared:
Testing Start Date: 0.5 mL fill

| Time | | | | | | | | | | % Acidic |
|---|---|---|---|---|---|---|---|---|---|---|
| Point/ | | Appearance, | Conc. | Turbidity | % | % | % | % | SEC. Ref Std & | % Main |
| Temp | Vial | pH | (mg/mL) | A360 nm | Monomer | Dimer | Aggregate | LMW | Avg. area count | % Basic |
| 0 | 1 | clear, sl opal, | 41.8 | 0.035 | 98.41 | 0.98 | 0.60 | — | RS007-001 | 19.4 |
| | | colorless, | 41.8 | | | | | | 33834 | 69.1 |
| | | 6.11 | | | | | | | | 11.5 |
| 4 wk, | 1 | v. sl opal, | N/A | 0.049 | 98.45 | 0.98 | 0.53 | 0.03 | RS007-001 | N/A |
| 5 C. | | colorless | | | | | | | 33626 | |
| | 2 | v. sl opal, | | 0.049 | 98.45 | 0.99 | 0.53 | 0.03 | | |
| | | colorless | | | | | | | | |
| 8 wk, | 1 | sl opal, colorless | | 0.051 | 98.61 | 0.95 | 0.44 | — | RS007-001 | N/A |
| 5 C. | 2 | sl opal, colorless | | 0.057 | 98.59 | 0.96 | 0.45 | — | 33670 | |
| 12 wk, | 1 | sl opal, colorless | | 0.054 | 98.37 | 1.06 | 0.46 | 0.11 | REF025 | N/A |
| 5 C. | 2 | sl opal, colorless | | 0.052 | 98.39 | 1.06 | 0.46 | 0.10 | (RS007-001) | |
| | | | | | | | | | 33608 | |
| 6 mo, | 1 | sl opal, colorless | | 0.065 | 98.39 | 1.07 | 0.39 | 0.16 | REF025 | 26.4 |
| 5 C. | | | | | | | | | (RS007-001) | 62.5 |
| | | | | | | | | | 33633 | 11.0 |
| | 2 | sl opal, colorless | | 0.076 | 98.39 | 1.05 | 0.40 | 0.16 | | 26.3 |
| | | | | | | | | | | 62.8 |
| | | | | | | | | | | 11.0 |

30° C.

| Time | | | | | | | | | | % Acidic |
|---|---|---|---|---|---|---|---|---|---|---|
| Point/ | | Appearance, | Conc. | Turbidity | % | % | % | % | SEC. Ref Std & | % Main |
| Temp | Vial | pH | (mg/mL) | A360 nm | Monomer | Dimer | Aggregate | LMW | Avg. area count | % Basic |
| 0 | 1 | clear, sl opal, | 41.8 | 0.035 | 98.41 | 0.98 | 0.60 | — | RS007-001 | 19.4 |
| | | colorless, | 41.8 | | | | | | 33834 | 69.1 |
| | | 6.11 | | | | | | | | 11.5 |
| 3 wk, | 1 | sl opal, | N/A | 0.018 | 98.39 | 0.99 | 0.51 | 0.14 | RS007-001 | N/A |
| 30 C. | | colorless | | | | | | | 33758 | |
| | 2 | sl opal, | | 0.013 | 98.37 | 0.99 | 0.49 | 0.14 | | |
| | | colorless | | | | | | | | |
| 6 wk, | 1 | sl opal, | N/A | 0.060 | 98.54 | 0.93 | 0.43 | 0.10 | RS007-001 | N/A |
| 5 C. | | colorless | | | | | | | 33506 | |
| | 2 | sl opal, | | 0.067 | 98.55 | 0.93 | 0.42 | 0.10 | | |
| | | colorless | | | | | | | | |
| 9 wk, | 1 | sl opal, | N/A | 0.047 | 98.28 | 1.03 | 0.40 | 0.29 | RS007-001 | N/A |
| 5 C. | | colorless | | | | | | | 33775 | |
| | 2 | sl opal, | | 0.054 | 98.25 | 1.07 | 0.38 | 0.30 | | |
| | | colorless | | | | | | | | |
| 12 wk, | 1 | sl opal, | N/A | 0.074 | 98.13 | 1.12 | 0.37 | 0.38 | REF025 | N/A |
| 30 C. | | colorless | | | | | | | (RS007-001) | |
| | 2 | sl opal, | N/A | 0.063 | 98.16 | 1.09 | 0.38 | 0.37 | 33586 | N/A |
| | | colorless | | | | | | | | |

40° C.

| Time | | | | | | | | | | % Acidic |
|---|---|---|---|---|---|---|---|---|---|---|
| Point/ | | Appearance, | Conc. | Turbidity | % | % | % | % | SEC. Ref Std & | % Main |
| Temp | Vial | pH | (mg/mL) | A360 nm | Monomer | Dimer | Aggregate | LMW | Avg. area count | % Basic |
| 0 | 1 | clear, sl opal, | 41.8 | 0.035 | 98.41 | 0.98 | 0.60 | — | RS007-001 | 19.4 |
| | | colorless, | 41.8 | | | | | | 33834 | 69.1 |
| | | 6.11 | | | | | | | | 11.5 |
| 2 wk, | 1 | clear, sl opal, | N/A | 0.058 | 98.14 | 1.00 | 0.48 | 0.38 | RS007-001 | N/A |
| 40 C. | | colorless | | | | | | | 33732 | |
| | 2 | clear, sl opal, | | 0.054 | 98.39 | 0.93 | 0.45 | 0.24 | | |
| | | colorless | | | | | | | | |
| 4 wk, | 1 | sl opal, | N/A | 0.054 | 96.06 | 1.07 | 0.47 | 2.40 | RS007-001 | N/A |
| 40 C. | | colorless | | | | | | | 34124 | |
| | 2 | v. sl opal, | | 0.055 | 95.90 | 1.06 | .044 | 2.60 | | |
| | | colorless | | | | | | | | |
| 8 wk, | 1 | sl opal, | 41.3 | 0.065 | 91.64 | 1.23 | 0.72 | 6.41 | RS007-001 | N/A |
| 40 C. | | colorless | (potency = | | | | | | 33852 | |
| | | | 80.0%) | | | | | | | |
| | 2 | sl opal, | 43.1 | 0.064 | 91.75 | 1.26 | 0.70 | 6.30 | | |
| | | colorless | | | | | | | | |
| 12 wk, | 1 | sl opal, | N/A | 0.081 | 89.51 | 1.58 | 1.35 | 7.55 | REF025 | N/A |
| 40 C. | | colorless | | | | | | | (RS007-001) | |
| | 2 | sl opal, | | 0.083 | 89.66 | 1.61 | 1.38 | 7.35 | 33590 | |
| | | colorless | | | | | | | | |

TABLE 17
Reconstituted
200 mg/mL Natalizumab, 100 mg/mL Sucrose, 24 mM Histidine, 0.08% PS-80
Reconstitute with 0.45 mL DI water to get 0.5 mL total volume
Prepared:
Lyophilization Complete:
Testing Start Date:

| Time | | | Recon | | | | | | | | | % Acidic |
|---|---|---|---|---|---|---|---|---|---|---|---|---|
| Point/ | | Appearance, | Time | KF % | Conc. | Turbidity | % | % | % | % | SEC Ref Std & | % Main |
| Temp. | Vial | pH | (min.) | Moisture | (mg/mL) | A360 nm | Monomer | Dimer | Aggregate | LMW | Avg. area count | % Basic |
| Pre-lyo | 1 | clear, | N/A | N/A | 50.0 | 0.028 | 98.28 | 1.04 | 0.60 | 0.07 | RS007-001 | 20.7 |
| | | sl opal, | | | 51.5 | | | | | | 33834 | 68.1 |
| | | 6.08 | | | | | | | | | | 11.3 |
| 0 | 1 | clear | 13.5 | 4.0 | 183.2 | 0.160 | 98.14 | 1.11 | 0.69 | 0.06 | RS007-001 | 20.4 |
| | | | | | | | | | | | 33791 | 69.0 |
| | | | | | | | | | | | | 10.7 |
| | 2 | clear | 4.7 | | 169.8 | 0.134 | 98.14 | 1.10 | 0.69 | 0.07 | | 20.1 |
| | | | | | | | | | | | | 64.7 |
| | | | | | | | | | | | | 15.2 |
| 4 wk, | 1 | sl opal, | 8.5 | N/A | N/A | 0.131 | 98.03 | 1.16 | 0.69 | 0.12 | RS007-001 | N/A |
| 5 C. | | colorless | | | | | | | | | 33626 | |
| | 2 | sl opal, | 8.3 | | | 0.140 | 98.03 | 1.16 | 0.69 | 0.12 | | |
| | | colorless | | | | | | | | | | |
| 8 wk, | 1 | opal, | 6.0 | N/A | 180.0 | 0.131 | 98.32 | 1.10 | 0.58 | — | RS007-001 | 22.1 |
| 5 C. | | colorless | | | | | | | | | 33670 | 63.9 |
| | | | | | | | | | | | | 14.0 |
| | 2 | opal, | 4.1 | | 185.4 | 0.135 | 98.33 | 1.09 | 0.58 | — | | 22.9 |
| | | colorless | | | | | | | | | | 62.9 |
| | | | | | | | | | | | | 14.2 |
| 12 wk, | 1 | opal, | 4.7 | N/A | N/A | 0.144 | 97.99 | 1.28 | 0.64 | 0.10 | REF025 | N/A |
| 5 C. | | colorless | | | | | | | | | (RS007-001) | |
| | 2 | opal, | 5.5 | | | 0.147 | 97.99 | 1.28 | 0.64 | 0.10 | 33608 | |
| | | colorless | | | | | | | | | | |
| 6 mo | 1 | opal, | 9.8 | N/A | 176.0 | 0.133 | 98.24 | 1.25 | 0.51 | — | REF025 | 21.8 |
| 5 C. | | colorless | | | | | | | | | (RS007-001) | 66.2 |
| | | | | | | | | | | | 33633 | 12.0 |
| | 2 | opal, | 7.8 | | 172.6 | 0.157 | 98.19 | 1.28 | 0.53 | — | | 21.8 |
| | | colorless | | | | | | | | | | 66.2 |
| | | | | | | | | | | | | 12.0 |
| *12 mo, | 1 | opal, slightly | 13.2 | N/A | 222.8 | 0.113 | 97.76 | 1.46 | 0.68 | 0.10 | REF025 | 20.5 |
| 5 C. | | yellow | | | | | | | | | (RS007-001) | 65.3 |
| | | | | | | | | | | | 36188 | 14.2 |

30° C.

| Time | | | Recon | | | | | | | | | % Acidic |
|---|---|---|---|---|---|---|---|---|---|---|---|---|
| Point/ | | | Time | KF % | Conc. | Turbidity | % | % | % | % | SEC Ref Std & | % Main |
| Temp. | Vial | Appearance | (min.) | Moisture | (mg/mL) | A360 nm | Monomer | Dimer | Aggregate | LMW | Avg. area count | % Basic |
| 0 | 1 | clear, sl opal | 13.5 | 4.0 | 183.2 | 0.160 | 98.14 | 1.11 | 0.69 | 0.06 | RS007-001 | 20.4 |
| | | | | | | | | | | | 33791 | 69.0 |
| | | | | | | | | | | | | 10.7 |
| | 2 | clear, sl opal | 4.7 | | 169.8 | 0.134 | 98.14 | 1.10 | 0.69 | 0.07 | | 20.1 |
| | | | | | | | | | | | | 64.7 |
| | | | | | | | | | | | | 15.2 |
| 3 wk, | 1 | sl opal, | 3.3 | N/A | N/A | 0.165 | 97.50 | 1.68 | 0.68 | 0.14 | RS007-001 | N/A |
| 30 C. | | colorless | | | | | | | | | 33758 | |
| | 2 | sl opal, | 4.2 | | | 0.169 | 97.53 | 1.64 | 0.69 | 0.14 | | |
| | | colorless | | | | | | | | | | |
| 6 wk, | 1 | opal, | 4.4 | N/A | N/A | 0.175 | 97.31 | 1.88 | 0.68 | 0.13 | RS007-001 | N/A |
| 30 C. | | colorless | | | | | | | | | 33506 | |
| | 2 | opal, | 5.8 | | | 0.168 | 97.31 | 1.89 | 0.67 | 0.13 | | |
| | | colorless | | | | | | | | | | |
| 9 wk, | 1 | opal, | 6.5 | N/A | N/A | 0.181 | 96.94 | 2.71 | 0.24 | 0.11 | RS007-001 | N/A |
| 30 C. | | colorless | | | | | | | | | 33775 | |
| | 2 | opal, | 4.1 | | | 0.169 | 96.91 | 2.75 | 0.25 | 0.10 | | |
| | | colorless | | | | | | | | | | |
| 12 wk, | 1 | opal, | 6.8 | N/A | N/A | 0.175 | 96.68 | 2.49 | 0.73 | 0.11 | REF025 | N/A |
| 30 C. | | colorless | | | | | | | | | (RS007-001) | |
| | 2 | opal, | 5.6 | | | 0.184 | 96.71 | 2.47 | 0.72 | 0.09 | 33586 | |
| | | colorless | | | | | | | | | | |

40° C.

| Time | | | Recon | | | | | | | | | % Acidic |
|---|---|---|---|---|---|---|---|---|---|---|---|---|
| Point/ | | | Time | KF % | Conc. | Turbidity | % | % | % | % | SEC Ref Std & | % Main |
| Temp. | Vial | Appearance | (min.) | Moisture | (mg/mL) | A360 nm | Monomer | Dimer | Aggregate | LMW | Avg. area count | % Basic |
| 0 | 1 | clear, sl opal | 13.5 | 4.0 | 183.2 | 0.160 | 98.14 | 1.11 | 0.69 | 0.06 | RS007-001 | 20.4 |
| | | | | | | | | | | | 33791 | 69.0 |
| | | | | | | | | | | | | 10.7 |
| | 2 | clear, sl opal | 4.7 | | 169.8 | 0.134 | 98.14 | 1.10 | 0.69 | 0.07 | | 20.1 |
| | | | | | | | | | | | | 64.7 |
| | | | | | | | | | | | | 15.2 |
| 2 wk, | 1 | clear, sl opal | 3.3 | N/A | N/A | 0.179 | 96.83 | 2.30 | 0.71 | 0.16 | RS007-001 | N/A |
| 40 C. | 2 | clear, sl opal | 3.2 | | | 0.165 | 96.88 | 2.27 | 0.70 | 0.15 | 33732 | |
| 4 wk, | 1 | sl opal, | 9.5 | N/A | 168.0 | 0.172 | 96.21 | 2.80 | 0.85 | 0.15 | RS007-001 | N/A |
| 40 C. | | colorless | | | | | | | | | 34124 | |
| | 2 | sl opal, | 9.4 | | 162.6 | 0.184 | 96.17 | 2.84 | 0.84 | 0.15 | | |
| | | colorless | | | | | | | | | | |
| 8 wk, | 1 | opal, | 9.5 | N/A | 179.0 | 0.203 | 95.82 | 3.94 | 0.25 | — | RS007-001 | 24.6 |
| 40 C. | | colorless | | | | | | | | | 33852 | 55.6 |
| | | | | | | | | | | | | 19.8 |
| | 2 | opal, | 5.1 | | 179.4 | 0.214 | 95.80 | 3.38 | 0.82 | — | | 26.5 |
| | | colorless | | | | | | | | | | 58.8 |
| | | | | | | | | | | | | 14.7 |

*Only 1 vial available for analysis at 12-month time point. Also, cake appeared to be double in size, so reconstituted with double the diluent.

TABLE 18
Reconstituted
200 mg/mL Natalizumab, 100 mg/mL Sucrose, 24 mM Histidine, 60 mM NaCl,
0.08% PS-80 Reconstitute with 0.45 mL DI water to get 0.5 mL total volume
Prepared:
Lyophilization Complete:
Testing Start Date:

| Time | | | Recon | | | | | | | | | % Acidic |
|---|---|---|---|---|---|---|---|---|---|---|---|---|
| Point/ | | | Time | KF % | Conc. | Turbidity | % | % | % | % | SEC Ref Std & | % Main |
| Temp. | Vial | Appearance | (min.) | Moisture | (mg/mL) | A360 nm | Monomer | Dimer | Aggregate | LMW | Avg. area count | % Basic |
| 0 | 1 | clear | 4.6 | 3.3 | 184.2 | 0.269 | 98.21 | 1.10 | 0.61 | 0.08 | RS007-001 | 18.1 |
| | | | | | | | | | | | 33791 | 65.7 |
| | | | | | | | | | | | | 16.2 |
| | 2 | clear | 7.0 | | 182.0 | 0.273 | 98.20 | 1.11 | 0.61 | 0.08 | | 20.7 |
| | | | | | | | | | | | | 62.4 |
| | | | | | | | | | | | | 16.8 |
| 4 wk, | 1 | sl opal, | 8.6 | N/A | N/A | 0.291 | 98.16 | 1.13 | 0.61 | 0.10 | RS007-001 | N/A |
| 5 C. | | colorless | | | | | | | | | 33626 | |
| | 2 | Opal, | 8.2 | | | 0.510 | 98.14 | 1.16 | 0.62 | 0.08 | | |
| | | colorless | | | | | | | | | | |
| 8 wk, | 1 | v. opal, | 3.2 | N/A | 183.7 | 0.287 | 98.37 | 1.09 | 0.54 | — | RS007-001 | 22.6 |
| 5 C. | | colorless | | | | | | | | | 33670 | 63.1 |
| | | | | | | | | | | | | 14.3 |
| | 2 | v. opal, | 6.8 | | 184.0 | 0.509 | 98.35 | 1.09 | 0.55 | — | | 22.0 |
| | | colorless | | | | | | | | | | 64.0 |
| | | | | | | | | | | | | 14.0 |
| 12 wk, | 1 | v. opal, | 5.7 | N/A | N/A | 0.471 | 98.06 | 1.26 | 0.58 | 0.10 | REF025 | N/A |
| 5 C. | | colorless | | | | | | | | | (RS007-001) | |
| | 2 | v. opal, | 4.8 | | | 0.485 | 98.05 | 1.26 | 0.58 | 0.11 | 33608 | |
| | | colorless | | | | | | | | | | |
| 12 mo, | 1 | N/A | N/A | N/A | N/A | N/A | 97.86 | 1.45 | 0.60 | 0.10 | REF025 | N/A |
| 5 C. | 2 | | | | | | 97.66 | 1.65 | 0.58 | 0.12 | (RS007-001) | |
| | | | | | | | | | | | 36412 | |

30° C.

| Time | | | Recon | | | | | | | | | % Acidic |
|---|---|---|---|---|---|---|---|---|---|---|---|---|
| Point/ | | | Time | KF % | Conc. | Turbidity | % | % | % | % | SEC Ref Std & | % Main |
| Temp. | Vial | Appearance | (min.) | Moisture | (mg/mL) | A360 nm | Monomer | Dimer | Aggregate | LMW | Avg. area count | % Basic |
| 0 | 1 | clear | 4.6 | 3.3 | 184.2 | 0.269 | 98.21 | 1.10 | 0.61 | 0.08 | RS007-001 | 18.1 |
| | | | | | | | | | | | 33791 | 65.7 |
| | | | | | | | | | | | | 16.2 |
| | 2 | clear | 7.0 | | 182.0 | 0.273 | 98.20 | 1.11 | 0.61 | 0.08 | | 20.7 |
| | | | | | | | | | | | | 62.4 |
| | | | | | | | | | | | | 16.8 |
| 3 wk, | 1 | opal, | 6.6 | N/A | N/A | 0.376 | 97.65 | 1.61 | 0.62 | 0.12 | RS007-001 | N/A |
| 30 C. | | colorless | | | | | | | | | 33758 | |
| | 2 | opal, | 5.8 | | | 0.444 | 97.60 | 1.66 | 0.63 | 0.12 | | |
| | | colorless | | | | | | | | | | |
| 6 wk, | 1 | v. opal, | 6.0 | N/A | N/A | 0.436 | 97.24 | 2.00 | 0.65 | 0.10 | RS007-001 | N/A |
| 30 C. | | colorless | | | | | | | | | 33506 | |
| | 2 | v. opal, | 7.2 | | | 0.373 | 97.31 | 1.95 | 0.64 | 0.10 | | |
| | | colorless | | | | | | | | | | |
| 9 wk, | 1 | v. opal, | 6.2 | N/A | N/A | 0.443 | 97.07 | 2.20 | 0.59 | 0.14 | RS007-001 | N/A |
| 30 C. | | colorless | | | | | | | | | 33775 | |
| | 2 | v. opal, | 12.4 | | | 0.402 | 97.07 | 2.19 | 0.59 | 0.14 | | |
| | | colorless | | | | | | | | | | |
| 12 wk, | 1 | v. opal, | 6.2 | N/A | N/A | 0.401 | 96.91 | 2.34 | 0.64 | 0.11 | REF025 | N/A |
| 30 C. | | colorless | | | | | | | | | (RS007-001) | |
| | 2 | v. opal, | 8.1 | | | 0.460 | 96.75 | 2.47 | 0.67 | 0.11 | 33586 | |
| | | colorless | | | | | | | | | | |
| 12 mo | 1 | N/A | N/A | N/A | N/A | N/A | 94.99 | 3.98 | 0.90 | 0.13 | REF025 | N/A |
| 30 C. | 2 | | | | | | | | | | (RS007-001) | |
| | | | | | | | 95.19 | 3.74 | 0.93 | 0.14 | 36412 | |

40° C.

| Time | | | Recon | | | | | | | | | % Acidic |
|---|---|---|---|---|---|---|---|---|---|---|---|---|
| Point/ | | | Time | KF % | Conc. | Turbidity | % | % | % | % | SEC Ref Std & | % Main |
| Temp. | Vial | Appearance | (min.) | Moisture | (mg/mL) | A360 nm | Monomer | Dimer | Aggregate | LMW | Avg. area count | % Basic |
| 0 | 1 | clear | 4.6 | 3.3 | 184.2 | 0.269 | 98.21 | 1.10 | 0.61 | 0.08 | RS007-001 | 18.1 |
| | | | | | | | | | | | 33791 | 65.7 |
| | | | | | | | | | | | | 16.2 |
| | 2 | clear | 7.0 | | 182.0 | 0.273 | 98.20 | 1.11 | 0.61 | 0.08 | | 20.7 |
| | | | | | | | | | | | | 62.4 |
| | | | | | | | | | | | | 16.8 |
| 2 wk, | 1 | clear, sl opal | 5.2 | N/A | N/A | 0.322 | 96.97 | 2.23 | 0.66 | 0.13 | RS007-001 | N/A |
| 40 C. | 2 | clear, sl opal | 7.3 | | | 0.328 | 97.06 | 2.17 | 0.64 | 0.13 | 33732 | |
| 4 wk, | 1 | sl opal, | 8.9 | N/A | 169.0 | 0.428 | 96.32 | 2.75 | 0.79 | 0.15 | RS007-001 | N/A |
| 40 C. | | colorless | | | | | | | | | 34124 | |
| | 2 | sl opal, | 4.4 | | 174.2 | 0.332 | 96.53 | 2.62 | 0.73 | 0.12 | | |
| | | colorless | | | | | | | | | | |
| 8 wk, | 1 | v. opal, | 9.8 | N/A | 164.6 | 0.339 | 96.21 | 3.09 | 0.70 | — | RS007-001 | 22.6 |
| 40 C. | | colorless | | | | | | | | | 33852 | 59.3 |
| | | | | | | | | | | | | 18.1 |
| | 2 | v. opal, | 12.2 | | 180.0 | 0.364 | 95.55 | 3.62 | 0.83 | — | | 24.0 |
| | | colorless | | | | | | | | | | 51.5 |
| | | | | | | | | | | | | 24.5 |
| 12 wk | 1 | opal, | 6.5 | N/A | N/A | 0.359 | 94.92 | 4.03 | 0.93 | 0.12 | REF025 | N/A |
| 40 C. | | colorless | | | | | | | | | (RS007-001) | |
| | 2 | opal, | 11.2 | | | 0.397 | 94.31 | 4.51 | 1.08 | 0.11 | 33590 | |
| | | colorless | | | | | | | | | | |

TABLE 19
Reconstituted
200 mg/mL Natalizumab, 100 mg/mL Sucrose, 24 mM Histidine, 0.08% PS-20
Reconstitute with 0.45 mL DI water to get 0.5 mL total volume
Prepared:
Lyophilization Complete:
Testing Start Date:

| Time | | | Recon | | | | | | | | | % Acidic |
|---|---|---|---|---|---|---|---|---|---|---|---|---|
| Point/ | | | Time | KF % | Conc. | Turbidity | % | % | % | % | SEC Ref Std & | % Main |
| Temp. | Vial | Appearance | (min.) | Moisture | (mg/mL) | A360 nm | Monomer | Dimer | Aggregate | LMW | Avg. area count | % Basic |
| 0 | 1 | clear | 5.1 | 4.3 | 173.3 | 0.169 | 98.04 | 1.12 | 0.78 | 0.07 | RS007-001 | 19.4 |
| | | | | | | | | | | | 33791 | 69.5 |
| | | | | | | | | | | | | 11.1 |
| | 2 | clear | 5.8 | | 165.4 | 0.161 | 98.05 | 1.12 | 0.77 | 0.06 | | 20.7 |
| | | | | | | | | | | | | 62.9 |
| | | | | | | | | | | | | 16.4 |
| 4 wk, | 1 | v. sl opal, | 8.2 | N/A | N/A | 0.143 | 97.95 | 1.16 | 0.78 | 0.11 | RS007-001 | N/A |
| 5 C. | | colorless | | | | | | | | | 33626 | |
| | 2 | v. sl opal, | 11.4 | | | 0.184 | 97.92 | 1.17 | 0.79 | 0.13 | | |
| | | colorless | | | | | | | | | | |
| 8 wk, | 1 | opal, | 6.0 | N/A | 180.0 | 0.160 | 98.25 | 1.11 | 0.64 | — | RS007-001 | 24.2 |
| 5 C. | | colorless | | | | | | | | | 33670 | 61.1 |
| | | | | | | | | | | | | 14.7 |
| | 2 | opal, | 6.7 | | 178.5 | 0.154 | 98.24 | 1.11 | 0.65 | — | | 23.5 |
| | | colorless | | | | | | | | | | 62.3 |
| | | | | | | | | | | | | 14.2 |
| 12 wk, | 1 | opal, | 5.5 | N/A | N/A | 0.154 | 97.89 | 1.29 | 0.73 | 0.09 | REF025 | N/A |
| 5 C. | | colorless | | | | | | | | | (RS007-001) | |
| | 2 | opal, | 4.6 | | | 0.163 | 97.90 | 1.28 | 0.72 | 0.10 | 33608 | |
| | | colorless | | | | | | | | | | |
| 12 mo, | 1 | N/A | N/A | N/A | N/A | N/A | 97.69 | 1.47 | 0.74 | 0.10 | REF025 | N/A |
| 5 C. | 2 | | | | | | 97.68 | 1.45 | 0.77 | 0.10 | (RS007-001) | |
| | | | | | | | | | | | 36412 | |

30° C.

| Time | | | Recon | | | | | | | | | % Acidic |
|---|---|---|---|---|---|---|---|---|---|---|---|---|
| Point/ | | | Time | KF % | Conc. | Turbidity | % | % | % | % | SEC Ref Std & | % Main |
| Temp. | Vial | Appearance | (min.) | Moisture | (mg/mL) | A360 nm | Monomer | Dimer | Aggregate | LMW | Avg. area count | % Basic |
| 0 | 1 | clear | 5.1 | 4.3 | 173.3 | 0.169 | 98.04 | 1.12 | 0.78 | 0.07 | RS007-001 | 19.4 |
| | | | | | | | | | | | 33791 | 69.5 |
| | | | | | | | | | | | | 11.1 |
| | 2 | clear | 5.8 | | 165.4 | 0.161 | 98.05 | 1.12 | 0.77 | 0.06 | | 20.7 |
| | | | | | | | | | | | | 62.9 |
| | | | | | | | | | | | | 16.4 |
| 3 wk, | 1 | sl opal, | >10* | N/A | N/A | 0.162 | 97.65 | 1.40 | 0.81 | 0.14 | RS007-001 | N/A |
| 30 C. | | colorless | | | | | | | | | 33758 | |
| | 2 | sl opal, | 5.3 | | | 0.175 | 97.41 | 1.68 | 0.79 | 0.13 | | |
| | | colorless | | | | | | | | | | |
| 6 wk, | 1 | opal, | 8.5 | N/A | N/A | 0.176 | 97.18 | 1.91 | 0.78 | 0.12 | RS007-001 | N/A |
| 30 C. | | colorless | | | | | | | | | 33506 | |
| | 2 | opal, | 6.9 | | | | 0.202 | 97.12 | 1.96 | 0.79 | 0.13 | |
| | | colorless | | | | | | | | | | |
| 9 wk, | 1 | opal, | 4.7 | N/A | N/A | 0.196 | 97.15 | 2.14 | 0.71 | — | RS007-001 | N/A |
| 30 C. | | colorless | | | | | | | | | 33775 | |
| | 2 | opal, | 3.8 | | | 0.195 | 97.04 | 2.25 | 0.71 | — | | |
| | | colorless | | | | | | | | | | |
| 12 wk, | 1 | opal, | 5.9 | N/A | N/A | 0.203 | 96.55 | 2.54 | 0.82 | 0.09 | REF025 | N/A |
| 30 C. | | colorless | | | | | | | | | (RS007-001) | |
| | 2 | opal, | 6.1 | | | 0.195 | 96.64 | 2.45 | 0.81 | 0.10 | 33586 | |
| | | colorless | | | | | | | | | | |
| 12 mo, | 1 | N/A | N/A | N/A | N/A | N/A | 94.62 | 3.99 | 1.26 | 0.13 | REF025 | N/A |
| 30 C. | | | | | | | | | | | (RS007-001) | |
| | 2 | | | | | | 94.67 | 4.08 | 1.16 | 0.10 | 36412 | |

40° C.

| Time | | | Recon | | | | | | | | | % Acidic |
|---|---|---|---|---|---|---|---|---|---|---|---|---|
| Point/ | | | Time | KF % | Conc. | Turbidity | % | % | % | % | SEC Ref Std & | % Main |
| Temp. | Vial | Appearance | (min.) | Moisture | (mg/mL) | A360 nm | Monomer | Dimer | Aggregate | LMW | Avg. area count | % Basic |
| 0 | 1 | clear | 5.1 | 4.3 | 173.3 | 0.169 | 98.04 | 1.12 | 0.78 | 0.07 | RS007-001 | 19.4 |
| | | | | | | | | | | | 33791 | 69.5 |
| | | | | | | | | | | | | 11.1 |
| | 2 | clear | 5.8 | | 165.4 | 0.161 | 98.05 | 1.12 | 0.77 | 0.06 | | 20.7 |
| | | | | | | | | | | | | 62.9 |
| | | | | | | | | | | | | 16.4 |
| 2 wk, | 1 | clear, sl opal | 6.6 | N/A | N/A | 0.185 | 96.80 | 2.25 | 0.81 | 0.14 | RS007-001 | N/A |
| 40 C. | 2 | clear, sl opal | 4.5 | | | 0.184 | 96.82 | 2.24 | 0.81 | 0.13 | 33732 | |
| 4 wk, | 1 | v. sl opal, | 5.1 | N/A | 165.6 | 0.187 | 96.12 | 2.76 | 0.97 | 0.15 | RS007-001 | N/A |
| 40 C. | | colorless | | | | | | | | | 34124 | |
| | 2 | v. sl opal, | 6.1 | | 151.5 | 0.189 | 96.13 | 2.76 | 0.97 | 0.14 | | |
| | | colorless | | | | | | | | | | |
| 8 wk, | 1 | opal, | 5.9 | N/A | 177.3 | 0.228 | 95.83 | 3.28 | 0.89 | — | RS007-001 | 23.2 |
| 40 C. | | colorless | | | | | | | | | 33852 | 57.7 |
| | | | | | | | | | | | | 19.2 |
| | 2 | opal, | 7.1 | | 175.2 | 0.217 | 95.84 | 3.28 | 0.88 | — | | 22.8 |
| | | colorless | | | | | | | | | | 58.8 |
| | | | | | | | | | | | | 18.5 |
| 12 wk, | 1 | opal, | 6.5 | N/A | N/A | 0.257 | 94.71 | 4.06 | 1.14 | 0.10 | REF025 | N/A |
| 40 C. | | colorless | | | | | | | | | (RS007-001) | |
| | 2 | opal, | 6.5 | | | 0.255 | 94.77 | 4.01 | 1.12 | 0.10 | 33590 | |
| | | colorless | | | | | | | | | | |

*Vial had crack

TABLE 20
Reconstituted
200 mg/mL Natalizumab, 100 mg/mL Trehalose, 24 mM Histidine, 0.08% PS-80
Reconstitute with 0.45 mL DI water to get 0.5 mL total volume
Prepared:
Lyophilization Complete:
Testing Start Date:

| Time | | | Recon | | | | | | | | | % Acidic |
|---|---|---|---|---|---|---|---|---|---|---|---|---|
| Point/ | | | Time | KF % | Conc. | Turbidity | % | % | % | % | SEC Ref Std & | % Main |
| Temp. | Vial | Appearance | (min.) | Moisture | (mg/mL) | A360 nm | Monomer | Dimer | Aggregate | LMW | Avg. area count | % Basic |
| 0 | 1 | Clear | 6.5 | 5.4 | 167.2 | 0.152 | 98.03 | 1.14 | 0.77 | 0.07 | RS007-001 | 20.8 |
| | | | | | | | | | | | 33791 | 63.5 |
| | | | | | | | | | | | | 15.6 |
| | 2 | Clear | 8.9 | | 157.4 | 0.150 | 98.05 | 1.14 | 0.76 | 0.06 | | 20.5 |
| | | | | | | | | | | | | 64.4 |
| | | | | | | | | | | | | 15.2 |
| 4 wk, | 1 | v. sl opal, | 7.9 | N/A | N/A | 0.142 | 97.86 | 1.25 | 0.79 | 0.11 | RS007-001 | N/A |
| 5 C. | | colorless | | | | | | | | | 33626 | |
| | 2 | v. sl opal, | 9.7 | | | 0.140 | 97.89 | 1.23 | 0.77 | 0.11 | | |
| | | colorless | | | | | | | | | | |
| 8 wk, | 1 | opal, | 6.4 | N/A | 180.0 | 0.156 | 98.17 | 1.20 | 0.64 | — | RS007-001 | 22.4 |
| 5 C. | | colorless | | | | | | | | | 33670 | 63.3 |
| | | | | | | | | | | | | 14.3 |
| | 2 | opal, | 5.5 | | 183.4 | 0.151 | 98.16 | 1.21 | 0.63 | — | | 23.2 |
| | | colorless | | | | | | | | | | 62.2 |
| | | | | | | | | | | | | 14.5 |
| 12 wk, | 1 | opal, | 6.5 | N/A | N/A | 0.160 | 97.80 | 1.39 | 0.70 | 0.10 | REF025 | N/A |
| 5 C. | | colorless | | | | | | | | | (RS007-001) | |
| | 2 | opal, | 3.5 | | | 0.171 | 97.82 | 1.38 | 0.70 | 0.10 | 33608 | |
| | | colorless | | | | | | | | | | |
| 12 mo, | 1 | N/A | N/A | N/A | N/A | N/A | 97.39 | 1.75 | 0.73 | 0.13 | REF025 | N/A |
| 5 C. | 2 | | | | | | 97.38 | 1.75 | 0.76 | 0.11 | (RS007-001) | |
| | | | | | | | | | | | 36412 | |

30° C.

| Time | | | Recon | | | | | | | | | % Acidic |
|---|---|---|---|---|---|---|---|---|---|---|---|---|
| Point/ | | | Time | KF % | Conc. | Turbidity | % | % | % | % | SEC Ref Std & | % Main |
| Temp. | Vial | Appearance | (min.) | Moisture | (mg/mL) | A360 nm | Monomer | Dimer | Aggregate | LMW | Avg. area count | % Basic |
| 0 | 1 | Clear | 6.5 | 5.4 | 167.2 | 0.152 | 98.03 | 1.14 | 0.77 | 0.07 | RS007-001 | 20.8 |
| | | | | | | | | | | | 33791 | 63.5 |
| | | | | | | | | | | | | 15.6 |
| | 2 | clear | 8.9 | | 157.4 | 0.150 | 98.05 | 1.14 | 0.76 | 0.06 | | 20.5 |
| | | | | | | | | | | | | 64.4 |
| | | | | | | | | | | | | 15.2 |
| 3 wk, | 1 | sl opal, | 10.2 | N/A | N/A | 0.177 | 96.87 | 2.19 | 0.82 | 0.13 | RS007-001 | N/A |
| 30 C. | | colorless | | | | | | | | | 33758 | |
| | 2 | sl opal, | 6.3 | | | 0.173 | 96.88 | 2.18 | 0.81 | 0.13 | | |
| | | colorless | | | | | | | | | | |
| 6 wk, | 1 | opal, | 8.9 | N/A | N/A | 0.219 | 96.44 | 2.62 | 0.81 | 0.13 | RS007-001 | N/A |
| 30 C. | | colorless | | | | | | | | | 33506 | |
| | 2 | opal, | 7.6 | | | 0.200 | 96.41 | 2.66 | 0.80 | 0.13 | | |
| | | colorless | | | | | | | | | | |
| 9 wk, | 1 | opal, | 8.5 | N/A | N/A | 0.227 | 95.79 | 3.22 | 0.87 | 0.12 | RS007-001 | N/A |
| 30 C. | | colorless | | | | | | | | | 33775 | |
| | 2 | opal, | 10.4 | | | 0.240 | 95.71 | 3.29 | 0.87 | 0.13 | | |
| | | colorless | | | | | | | | | | |
| 12 wk, | 1 | opal, | 9.5 | N/A | N/A | 0.255 | 95.21 | 3.74 | 0.95 | 0.10 | REF025 | N/A |
| 30 C. | | colorless | | | | | | | | | (RS007-001) | |
| | 2 | opal, | 7.7 | | | 0.268 | 95.19 | 3.74 | 0.97 | 0.10 | 33586 | |
| | | colorless | | | | | | | | | | |
| 12 mo, | 1 | N/A | N/A | N/A | N/A | N/A | 91.45 | 6.75 | 1.69 | 0.11 | REF025 | N/A |
| 30 C. | 2 | | | | | | 91.53 | 6.74 | 1.62 | 0.11 | (RS007-001) | |
| | | | | | | | | | | | 36412 | |

40° C.

| Time | | | Recon | | | | | | | | | % Acidic |
|---|---|---|---|---|---|---|---|---|---|---|---|---|
| Point/ | | | Time | KF % | Conc. | Turbidity | % | % | % | % | SEC Ref Std & | % Main |
| Temp. | Vial | Appearance | (min.) | Moisture | (mg/mL) | A360 nm | Monomer | Dimer | Aggregate | LMW | Avg. area count | % Basic |
| 0 | 1 | clear | 6.5 | 5.4 | 167.2 | 0.152 | 98.03 | 1.14 | 0.77 | 0.07 | RS007-001 | 20.8 |
| | | | | | | | | | | | 33791 | 63.5 |
| | | | | | | | | | | | | 15.6 |
| | 2 | clear | 8.9 | | 157.4 | 0.150 | 98.05 | 1.14 | 0.76 | 0.06 | | 20.5 |
| | | | | | | | | | | | | 64.4 |
| | | | | | | | | | | | | 15.2 |
| 2 wk, | 1 | clear, sl opal | 10.1 | N/A | N/A | 0.239 | 95.50 | 3.45 | 0.89 | 0.15 | RS007-001 | N/A |
| 40 C. | 2 | clear, sl opal | 12.2 | | | 0.245 | 95.41 | 3.54 | 0.92 | 0.13 | 33732 | |
| 4 wk, | 1 | sl opal, | 7.5 | N/A | 164.1 | 0.295 | 93.80 | 4.85 | 1.19 | 0.15 | RS007-001 | N/A |
| 40 C. | | colorless | | | | | | | | | 34124 | |
| | 2 | sl opal, | 7.2 | | 175.0 | 0.301 | 93.91 | 4.72 | 1.24 | 0.14 | | |
| | | colorless | | | | | | | | | | |
| 8 wk, | 1 | opal, | 6.7 | N/A | 173.2 | 0.424 | 92.75 | 5.95 | 1.30 | — | RS007-001 | 28.9 |
| 40 C. | | colorless | | | (potency = | | | | | | 33852 | 49.9 |
| | | | | | 122.4%) | | | | | | | 21.1 |
| | 2 | opal, | 7.0 | | 171.7 | 0.428 | 92.70 | 5.96 | 1.34 | — | | 26.9 |
| | | colorless | | | | | | | | | | 53.8 |
| | | | | | | | | | | | | 19.3 |
| 12 wk, | 1 | v. opal, | 7.9 | N/A | N/A | 0.546 | 90.63 | 7.44 | 1.82 | 0.11 | REF025 | N/A |
| 40 C. | | colorless | | | | | | | | | (RS007-001) | |
| | 2 | v. opal, | 9.7 | | | 0.580 | 90.67 | 7.43 | 1.79 | 0.11 | 33590 | |
| | | colorless | | | | | | | | | | |

TABLE 21
Reconstituted
160 mg/mL Natalizumab, 100 mg/mL Sucrose, 24 mM Histidine, 0.08% PS-80
Reconstitute with 0.45 mL DI water to get 0.5 mL total volume
Prepared:
Lyophilization Complete:
Testing Start Date:

| Time | | | Recon | | | | | | | | | % Acidic |
|---|---|---|---|---|---|---|---|---|---|---|---|---|
| Point/ | | | Time | KF % | Conc. | Turbidity | % | % | % | % | SEC Ref Std & | % Main |
| Temp. | Vial | Appearance | (min.) | Moisture | (mg/mL) | A360 nm | Monomer | Dimer | Aggregate | LMW | Avg. area count | % Basic |
| 0 | 1 | clear | 2.8 | 3.7 | 119.3 | 0.186 | 98.11 | 1.08 | 0.73 | 0.08 | RS007-001 | 19.2 |
| | | | | | | | | | | | 33791 | 69.2 |
| | | | | | | | | | | | | 11.6 |
| | 2 | clear | 3.6 | | 124.5 | 0.153 | 98.11 | 1.08 | 0.74 | 0.08 | | 21.0 |
| | | | | | | | | | | | | 63.4 |
| | | | | | | | | | | | | 15.6 |
| 4 wk, | 1 | v. sl opal, | 2.7 | N/A | N/A | 0.138 | 98.00 | 1.12 | 0.74 | 0.14 | RS007-001 | N/A |
| 5 C. | | colorless | | | | | | | | | 33626 | |
| | 2 | v. sl opal, | 2.4 | | | 0.149 | 98.00 | 1.12 | 0.74 | 0.14 | | |
| | | colorless | | | | | | | | | | |
| 8 wk, | 1 | opal, | 3.0 | N/A | 154.7 | 0.146 | 98.38 | 1.03 | 0.59 | — | RS007-001 | 22.1 |
| 5 C. | | colorless | | | (potency = | | | | | | 33670 | 65.3 |
| | | | | | 133%) | | | | | | | 12.5 |
| | 2 | opal, | 4.7 | | 150.7 | 0.163 | 98.39 | 1.03 | 0.58 | — | | 22.6 |
| | | colorless | | | | | | | | | | 63.7 |
| | | | | | | | | | | | | 13.7 |
| 12 wk, | 1 | opal, | 3.3 | N/A | N/A | 0.160 | 98.04 | 1.20 | 0.67 | 0.09 | REF025 | N/A |
| 5 C. | | colorless | | | | | | | | | (RS007-001) | |
| | 2 | opal, | 2.9 | | | 0.158 | 98.02 | 1.21 | 0.68 | 0.09 | 33608 | |
| | | colorless | | | | | | | | | | |
| 6 mo, | 1 | opal, | 3.9 | N/A | 147.9 | 0.168 | 98.21 | 1.19 | 0.59 | — | REF025 | 21.9 |
| 5 C. | | colorless | | | | | | | | | (RS007-001) | 66.2 |
| | | | | | | | | | | | 33633 | 11.9 |
| | 2 | opal, | 3.1 | | 155.2 | 0.164 | 98.20 | 1.21 | 0.59 | — | | 21.9 |
| | | colorless | | | | | | | | | | 66.2 |
| | | | | | | | | | | | | 11.9 |
| 12 mo, | 1 | opal, | 3.4 | N/A | 145.6 | 0.155 | 98.13 | 1.23 | 0.64 | — | REF025 | 20.9 |
| 5 C. | | colorless | | | | | | | | | (RS007-001) | 65.2 |
| | | | | | | | | | | | 36188 | 13.9 |
| | 2 | opal, | 2.7 | | 142.9 | 0.154 | 98.09 | 1.25 | 0.65 | — | | 20.2 |
| | | colorless | | | | | | | | | | 65.6 |
| | | | | | | | | | | | | 14.2 |

30° C.

| Time | | | Recon | | | | | | | | | % Acidic |
|---|---|---|---|---|---|---|---|---|---|---|---|---|
| Point/ | | | Time | KF % | Conc. | Turbidity | % | % | % | % | SEC Ref Std & | % Main |
| Temp. | Vial | Appearance | (min.) | Moisture | (mg/mL) | A360 nm | Monomer | Dimer | Aggregate | LMW | Avg. area count | % Basic |
| 0 | 1 | clear | 2.8 | 3.7 | 119.3 | 0.186 | 98.11 | 1.08 | 0.73 | 0.08 | RS007-001 | 19.2 |
| | | | | | | | | | | | 33791 | 69.2 |
| | | | | | | | | | | | | 11.6 |
| | 2 | clear | 3.6 | | 124.5 | 0.153 | 98.11 | 1.08 | 0.74 | 0.08 | | 21.0 |
| | | | | | | | | | | | | 63.4 |
| | | | | | | | | | | | | 15.6 |
| 3 wk, | 1 | sl opal, | 2.1 | N/A | N/A | 0.176 | 97.97 | 1.38 | 0.65 | — | RS007-001 | N/A |
| 30 C. | | colorless | | | | | | | | | 33758 | |
| | 2 | sl opal, | 1.6 | | | 0.141 | 97.58 | 1.51 | 0.74 | 0.18 | | |
| | | colorless | | | | | | | | | | |
| 6 wk, | 1 | opal, | 3.1 | N/A | N/A | 0.195 | 97.80 | 1.56 | 0.64 | — | RS007-001 | N/A |
| 30 C. | | colorless | | | | | | | | | 33506 | |
| | 2 | opal, | 2.9 | | | 0.175 | 97.76 | 1.59 | 0.64 | — | | |
| | | colorless | | | | | | | | | | |
| 9 wk, | 1 | opal, | 3.3 | N/A | N/A | 0.188 | 97.23 | 1.94 | 0.71 | 0.12 | RS007-001 | N/A |
| 30 C. | | colorless | | | | | | | | | 33775 | |
| | 2 | opal, | 3.8 | | | 0.204 | 97.55 | 1.80 | 0.65 | — | | |
| | | colorless | | | | | | | | | | |
| 12 wk, | 1 | opal, | 3.9 | N/A | N/A | 0.188 | 97.33 | 2.41 | 0.27 | — | REF025 | N/A |
| 30 C. | | colorless | | | | | | | | | (RS007-001) | |
| | 2 | opal, | 3.5 | | | 0.180 | 97.32 | 2.41 | 0.27 | — | 33586 | |
| | | colorless | | | | | | | | | | |
| 6 mo, | 1 | opal, slightly | 2.6 | N/A | 144.6 | 0.252 | 96.63 | 3.03 | 0.35 | — | REF025 | 22.1 |
| 30 C. | | yellow | | | | | | | | | (RS007-001) | 63.7 |
| | | | | | | | | | | | 33633 | 14.2 |
| | 2 | opal, slightly | 3.2 | | 143.9 | 0.259 | 96.60 | 3.01 | 0.39 | — | | 22.2 |
| | | yellow | | | | | | | | | | 63.6 |
| | | | | | | | | | | | | 14.2 |
| 12 mo, | 1 | opal, slightly | 6.0 | N/A | 140.8 | 0.218 | 96.07 | 2.99 | 0.94 | — | REF025 | 21.0 |
| 30 C. | | yellow | | | | | | | | | (RS007-001) | 62.6 |
| | | | | | | | | | | | 36188 | 16.5 |
| | 2 | opal, slightly | 4.5 | | 145.0 | 0.203 | 96.08 | 2.98 | 0.94 | — | | 21.0 |
| | | yellow | | | | | | | | | | 61.7 |
| | | | | | | | | | | | | 17.4 |

40° C.

| Time | | | Recon | | | | | | | | | % Acidic |
|---|---|---|---|---|---|---|---|---|---|---|---|---|
| Point/ | | | Time | KF % | Conc. | Turbidity | % | % | % | % | SEC Ref Std & | % Main |
| Temp. | Vial | Appearance | (min.) | Moisture | (mg/mL) | A360 nm | Monomer | Dimer | Aggregate | LMW | Avg. area count | % Basic |
| 0 | 1 | clear | 2.8 | 3.7 | 119.3 | 0.186 | 98.11 | 1.08 | 0.73 | 0.08 | RS007-001 | 19.2 |
| | | | | | | | | | | | 33791 | 69.2 |
| | | | | | | | | | | | | 11.6 |
| | 2 | clear | 3.6 | | 124.5 | 0.153 | 98.11 | 1.08 | 0.74 | 0.08 | | 21.0 |
| | | | | | | | | | | | | 63.4 |
| | | | | | | | | | | | | 15.6 |
| 2 wk, | 1 | clear, sl | 1.6 | N/A | N/A | 0.198 | 97.29 | 1.88 | 0.71 | 0.12 | RS007-001 | N/A |
| 40 C. | 2 | opal, | | | | | | | | | | |
| | | clear, sl | 2.3 | | | 0.171 | 97.32 | 1.87 | 0.70 | 0.12 | 33732 | |
| | | opal, | | | | | | | | | | |
| 4 wk, | 1 | sl opal, | 3.1 | N/A | 149.7 | 0.195 | 96.70 | 2.30 | 0.82 | 0.18 | RS007-001 | N/A |
| 40 C. | | colorless | | | | | | | | | 34124 | |
| | 2 | sl opal, | 1.8 | | 146.5 | 0.185 | 96.72 | 2.30 | 0.81 | 0.17 | | |
| | | colorless | | | | | | | | | | |
| 8 wk, | 1 | opal, | 3.9 | N/A | 145.5 | 0.228 | 96.63 | 3.12 | 0.25 | — | RS007-001 | 23.6 |
| 40 C. | | colorless | | | (potency = | | | | | | 33852 | 58.7 |
| | | | | | 144%) | | | | | | | 17.7 |
| | 2 | opal, | 2.1 | | 148.0 | 0.202 | 96.66 | 3.12 | 0.23 | — | | 23.5 |
| | | colorless | | | | | | | | | | 59.2 |
| | | | | | | | | | | | | 17.3 |
| 12 wk, | 1 | opal, | 3.4 | N/A | N/A | 0.225 | 95.69 | 3.27 | 0.94 | 0.10 | REF025 | N/A |
| 40 C. | | colorless | | | | | | | | | (RS007-001) | |
| | 2 | opal, | 2.5 | | | 0.220 | 95.70 | 3.27 | 0.94 | 0.09 | 33590 | |
| | | colorless | | | | | | | | | | |

### Example 3

As demonstrated below, three formulations with slightly different initial protein concentrations were successfully lyophilized and showed excellent stability when stored at 5° C. for 6 months. Stability of these formulations when stored at 40° C. for 8 weeks was comparable to stability seen previously. Stability of the pre-lyophilized formulations stored for 6 months at 5° C. and reconstituted solutions stored at either 5° or 25° for one week was also excellent. A starting concentration of 40 mg/mL showed slightly better stability and better reconstitution characteristics than the other formulations. However, reconstitution of this formulation did allow a deliverable dose of 1 mL at 150 mg/mL.

### Materials

The following table lists the materials and their source.

TABLE 22

| Item | Manufacturer |
|---|---|
| Bulk Natalizumab | Elan |
| (77.4 mg/mL) | |
| Sucrose (96 mg/mL) | Elan |
| 1% PS80 | Elan |
| 60 mM Histidine | Elan |
| 6 mM Histidine | Elan |
| 0.22-micron Cellulose | Corning |
| Acetate Filter | |
| 5 cc Vials | Kimble |
| Grey Butyl 20 mm | Kimble |
| Lyo Stoppers, 2 leg | |
| 20 mm Tear-Off | Wheaton |
| Aluminum Seals | |

TABLE 23
Equipment

HPLC
HPLC
Uv-Vis Spectrophotometer
Karl Fischer
Coulometric Titrator
pH Meter

### Natalizumab

Natalizumab used for this study was the same as that used in Study 3A and Study 3B (AQS-2190). Natalizumab used for these studies was prepared from natalizumab supplied by Biogenldec. The polysorbate 80 included in this material, which had been formulated and vialed, was removed prior to use in this formulation study. Briefly, material was subjected to diafiltration into a low ionic strength, high pH (10 mM tris, 10 mM NaCl, pH=8.5) buffer. The material was bound to a DEAE-Sepharose column under these conditions and then eluted using 10 mM sodium phosphate, 140 mM NaCl, pH 6. The column eluate was then diafiltered into 6 mM histidine, pH 6 and concentrated to between 70 and 100 mg/mL prior to further formulation.

### Chemicals and Reagents

All chemicals and reagents used in this study, except as noted were purchased from VWR and were ACS grade or better. USP grade reagents were used as excipients when available. Polysorbate 80 was purchased from Sigma (Cat # P6474); and was vegetable derived and low peroxide.

### Formulations

The following formulations were prepared by diluting stock solutions of the excipients and active to the desired concentrations. All formulations were at pH 6.

TABLE 24
Study 4 formulation parameters

| | | Target |
|---|---|---|
| Formulation | | Reconstituted |
| ID | Pre-lyo Concentrations | Concentration |
| 4089-iL | 40 mg/mL Ab, 24 mg/mL sucrose, 6 mM | 150 mg/mL |
| | his, 0.02% PS80 | Ab, 90 mg/mL |
| 4089-1M | 45 mg/mL Ab, 24 mg/mL sucrose, 6 mM | sucrose, 24 |
| | his, 0.02% PS80 | mM his, 0.08% |
| 4089-1N | 50 mg/mL Ab, 24 mg/mL sucrose, 6 mM | PS80 |
| | his, 0.02% PS80 | |

Abbreviations:
Ab = antibody,
his = histidine,
PS80 = polysorbate 80 2.2

### Methods

### Preparation of Formulations

The sample formulations were sterile filtered and filled as indicated into sterile glass vials. All samples for pre-lyophilized analysis were filled at 0.5 mL into 3 cc vials, stoppered and capped. Formulas to be lyophilized were filled at 4.0 mL into 5 cc Kimble vials.

### Lyophilization Cycle

During the freezing phase, the vials containing the samples were first cooled to 5° C. and held for 30 minutes. The temperature was then ramped to −5° C. over 20 minutes and held at −5° C. for 60 minutes. For the final stage of freezing, the vials were ramped to −40° C. over 45 minutes, held at this temperature for 2 hours, of which the last 20 minutes had a 100 mTorr vacuum applied. Primary drying was then performed as the temperature was taken to −25° C. over 20 min under vacuum of 100 mTorr and held there for 34 hours. The shelf temperature was ramped slowly to 20° C. over 10 hours to begin secondary drying at 100 mTorr, and then held at 20° C. for 6 hours.

### Study Set-Up

Vials of pre-lyophilized liquid and lyophilized cakes were placed at 5 and 40° C. Samples stored at 40° C. were assayed at 2, 4 and 8 weeks. Samples stored at 5° C. were assayed at 4 weeks and 6 months. At the 4 week time point, 4 vials from each storage temperature were reconstituted, after which 2 vials were placed at 5° C. and 2 vials were placed at 25° C., for 1 week of reconstitution stability. All formulations were assayed pre- and post-lyophilization at time zero.

### Assays

The samples were assayed by the following methods and the results are presented in tables 27-30. Not all assays were performed at all time points. With the exception of the zero time point for pre- and post-lyophilized samples, duplicate vials were sampled for each assay.

### Appearance by Visual Examination

All samples were examined visually and their appearance recorded. Lyophilized cakes were examined for color, uniformity, robustness and evidence of meltback. Liquid and reconstituted samples were examined for color, clarity and presence of particulates.

### Residual Moisture and Reconstitution Time

Lyophilized cakes were assayed for residual moisture using Karl Fischer Colorimetric Titrator at the zero time point. Reconstitution time was measured by adding the appropriate volume of DI water, followed by gentle swirling. The time for the cake to completely dissolve was recorded.

### Concentration

The concentration of all samples was measured diluting the samples to 1 mg/mL using natalizumab placebo. The UV absorbance was scanned from 400 to 250 nm using a Varian Cary 300Bio and 10 mm pathlength cuvette at 200 nm/min. The absorbance at lambda max was recorded and the concentration was determined by dividing that value by 1.498 (the absorptivity coefficient for natalizumab) and adjusting by the appropriate dilution.

### Turbidity

The absorption from 300 to 400 nm was measured of a neat sample using a 10 mm small volume cuvette (Starna Cells Inc. Cat #16.160-Q-10\Z20). The value recorded at 360 nm was reported.

### Size Exclusion Chromatography

The amount of monomer, high molecular weight aggregates, dimer and low molecular weight species in the samples were determined by size exclusion chromatography. Briefly, the samples were loaded onto the column neat and the load volumes adjusted to allow a mass of approximately 400 μg on the column. The mass of a reference material load was also adjusted to be comparable. Detection was recorded at both 215 and 280 nm, however the main peak was off scale at 215 nm, so the 280 nm trace was used for calculations. The results are presented in tables 27-30

### Results

Pre-Lyophilized Formulations

Tables 27-30 contain results recorded for all formulations. On visual examination, all formulations appeared colorless and slightly opalescent, without any visual particulates. The appearance did not change over time at 5° or 40° C. None of the samples showed a change in protein concentration within the variation of the analysis and no trend was observed. The turbidity of all formulations, as measured by absorbance at 360 nm, showed a slight increase after 6 months at 5° C. The 40° C. pre-lyophilized samples showed an increase of turbidity with time for all three formulations.

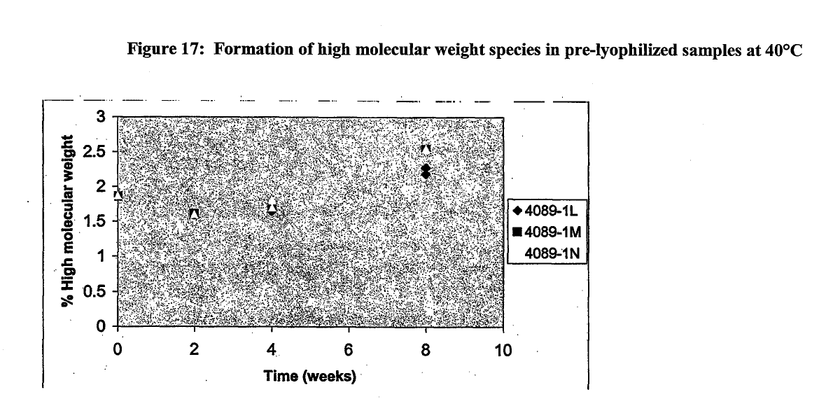

The formation of dimer, high molecular weight (aggregate) and low molecular weight species was monitored for all formulations. The results are shown in the tables and FIGS. 17-19.

FIG. 17 shows the formation of high molecular weight species at 40° C. The formation of high molecular weight species did not appear to begin until after 4 weeks of storage in the pre-lyophilized formulations. At 8 weeks a small increase in high molecular weight species were apparent.

At 5° C. there was no change in the level of high molecular weight species in any formulation for the 6 months of storage. This data is shown in the appendices but not graphed here.

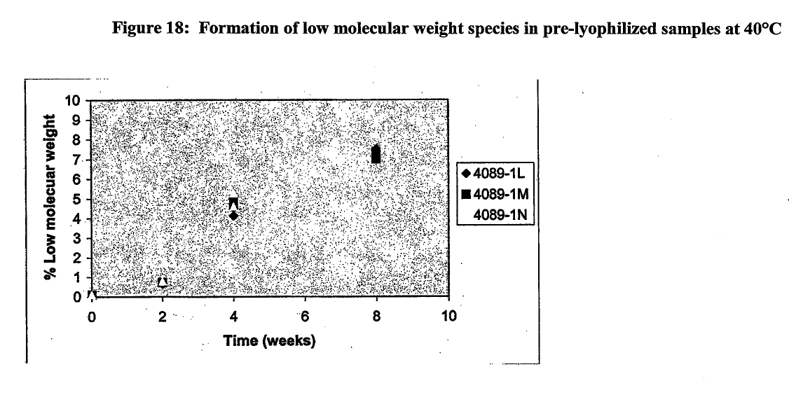

FIG. 18 shows the formation of low molecular weight species at 40° C. There was a definite trend in formation of low molecular weight species during storage at 40° C. Also, the formation of low molecular weight species appeared to proceed at a faster rate than the formation of high molecular weight species, making this an important degradation pathway in the liquid samples.

There was very little change in the percentage of low molecular weight species over a 6 month time period for the samples stored at 5° C. This data is shown in the appendices, but not graphed here. All of the pre-lyophilized formulations went from an initial level of approximately 0.11% to 0.18% at 6 months. BiogenIdec have shown the lower limit of quantitation for this assay to be 0.2%. Values below that the level are reported for trending information only.

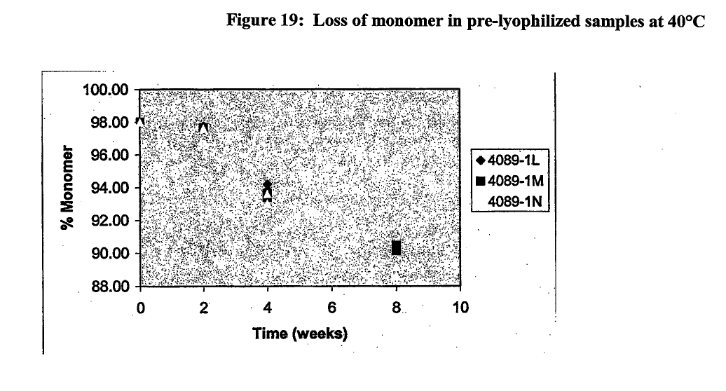

FIG. 19 shows the total loss of monomer at 40° C. due to formation of both high and low molecular weight species. At 5° C., there was essentially no change in the percent monomer. This would indicate that any of these formulations would be suitably stable as a pre-lyophilized formulation for storage up to 6 months at 2-8° C.

Selected samples were analyzed by cation exchange chromatography. In all cases the results showed a shift towards a more acidic species (deamidation) with storage based on time and temperature. There was no difference among the various pre-lyophilized formulations. Since deamidation is generally influenced by pH, ionic strength and certain buffer species, this result would be expected. This data is shown in the tables below.

All three pre-lyophilized formulations showed results similar to the pre-lyophilized formulations in Study 3A and 3B.

### Lyophilized Formulations

Table 25 below shows the reconstitution volumes tested in order to reach a protein concentration of 150 mg/mL. The reconstitution volumes determined to be closest to the target concentration were then used for the remainder of the study. The concentration was checked after each sample was completely dissolved. The reconstitution volumes used for 4089-1L, 4089-1M and 4089-1N, were 0.85, 1.0 and 1.1 mL, respectively.

TABLE 25
Reconstitution volumes

| | | Reconstitution | Concentration |
|---|---|---|---|
| Formulation | Vial | Volume (mL) | (mg/mL) |
| 4089-iL | 1 | 0.8 | 156.7 |
| | 2 | 0.9 | 143.9 |
| | 3 | 0.85 | 151.8 |
| 4089-1M | 1 | 0.8 | 168.2 |
| | 2 | 0.9 | 162.5 |
| | 3 | 1.0 | 158.5 |
| 4089-1N | 1 | 0.9 | 179.4 |
| | 2 | 1.1 | 146.1 |
| | | | 159.9 |
| | | | 154.4 |
| | | | 153.5 |
| | 3 | 1.1 | 160.9 |
| | | | 156.8 |

Table 26 below shows the ability to remove a 1 mL dose from the reconstituted samples. This was only possible with 4089-1N, which was reconstituted with 1.1 mL. These data indicate that to produce a final deliverable volume of 1 mL at 150 mg/mL, a fill volume of 4 mL with a protein concentration of 50 mg/mL would be necessary.

TABLE 26
Evaluation of a 1 mL dose

| | | | | Volume |
|---|---|---|---|---|
| | Reconstitution | | | Removed |
| Formulation | Volume (mL) | Needle Size | Vial | (mL) |
| 4089-1L | 0.85 | 21 gauge, 1½ | 1 | 0.75 |
| | | inch | 2 | 0.75 |
| | | | 3 | 0.85 |
| 4089-1M | 1.0 | 21 gauge, 1½ | 1 | 0.9 |
| | | inch | 2 | 0.8 |
| | | | 3 | 0.85 |
| 4089-1N | 1.1 | 20 gauge, 1½ | 1 | 1.0 |
| | | inch | 2 | 1.0 |
| | | | 3 | 1.0 |

### Stability

On visual examination, all formulations appeared to have acceptable lyophilized cakes, verifying that a 4 mL volume could be successfully lyophilized in a 5 mL vial. After reconstitution, all formulations appeared colorless and slightly opalescent, without any visible particulates. The appearance did not change over time at 5° or 40° C. None of the samples showed a change in protein concentration within the variation of the analysis and no trend was observed. The turbidity of all formulations, as measured by absorbance at 360 nm, showed a slight increase after 6 months at 5° C., from approximately 0.099-0.104 to 0.111-0.116. The 40° C. pre-lyophilized samples showed an increase of turbidity with time for all formulations.

The percent moisture of the lyophilized cakes ranged from 4.3 to 5.6 as measured by a Karl Fischer coulometer titrator. The average percent moisture for 4089-1L, 4089-1M and 4089-1N, was 4.5, 5.2 and 4.9, respectively. The moisture was relatively high in these samples.

The reconstitution times for this study were all below 17 minutes. FIG. 4 shows the reconstitution times at 40° C. There appears to be a slight trend toward increasing reconstitution times upon storage at 40° C. The samples with higher starting protein concentration also show a trend towards longer reconstitution times. This was also noted in earlier studies (Study A and B)

The formation of dimer, high molecular weight aggregate and low molecular weight species was monitored for all formulations. The tabular results are shown in the following tables.

FIG. 5 shows the formation of high molecular weight species at 40° C. There is a definite trend of formation of high molecular weight species during storage at this temperature. The rate of formation of the aggregate appeared to be slightly slower in formulation 4089-1L. Without wishing to be bound by theory, this could be due to either the lower protein concentration of the starting formulation or the slightly higher sucrose to protein ratio.

At 5° C. there was no change in the level of high molecular weight species in any formulation for the 6 months of storage which display the % HMW at various times.

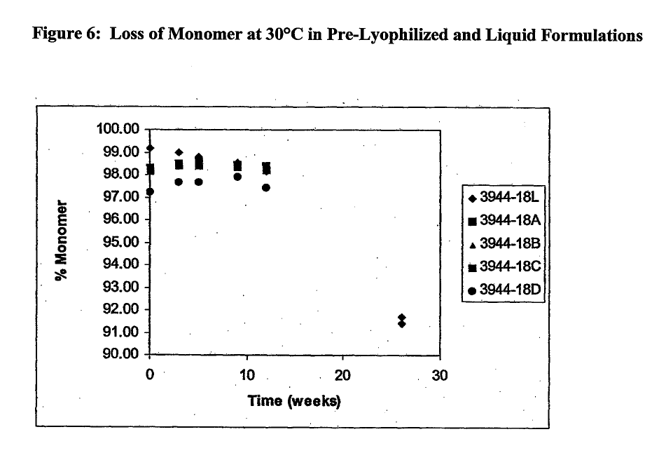

FIG. 6 shows the formation of low molecular weight species at 40° C. In contrast to the pre-lyophilized samples, which exhibited a rapid increase in the formation of low molecular weight species, the lyophilized samples showed no change over 8 weeks. There was no change in the percentage of low molecular weight species over a 6 month time period for the samples stored at 5° C. This data is shown in the appendices, but not graphed here.

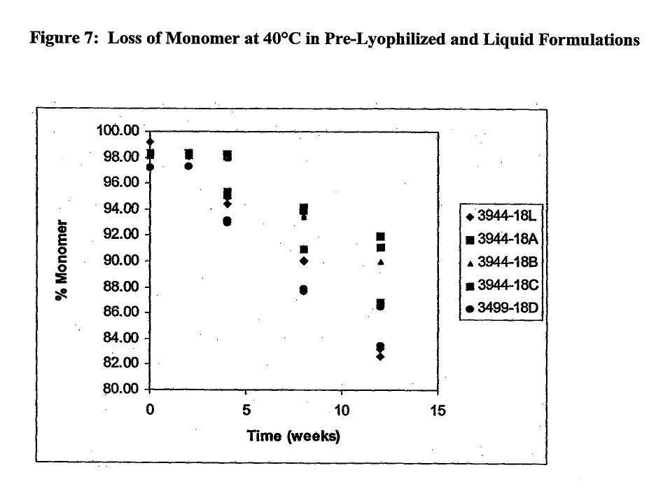

FIG. 7 shows the total loss of monomer at 40° C. due to formation of both high and low molecular weight species. At 5° C., there was essentially no change in the percent monomer. This would indicate that any of these formulations would be suitably stable as a lyophilized formulation for storage up to 6 months at 2-8° C. and longer.

Samples were also analyzed by cation exchange chromatography. In all cases the results showed a shift towards a more basic species after 8 weeks at 40° C. There is very little change in the charge distribution within the variability of the assay. Select samples also showed a slight shift towards a more acidic species at that time point. This data is shown in the tables below, in the final column as % Acidic, % Main and % Basic.

### Reconstituted Stability

After 4 weeks of storage at either 5° or 40° C., samples were reconstituted and then placed at 5° and 25° C. for 1 week of reconstituted stability. Testing was performed at the initial time point and at 1 week. The tables in the appendices contain all results recorded for all formulations. On visual examination, all formulations appeared colorless and slightly opalescent. All but two samples appeared free of particulates. The two vials containing “fluffy” white particulates had been stored at 40° C. prior to reconstitution and then stored at 25° C. for 1 week. The analyst may have contaminated these vials when they were opened for initial time point testing. None of the samples showed a change in protein concentration within the variation of the analysis and no trend was observed. The turbidity of all formulations, as measured by absorbance at 360 nm, did not show a change, within the variation of the analysis, after 1 week at either 5° or 25° C.

The formation of dimer, high molecular weight (aggregate) and low molecular weight species was monitored for all formulations. The results are shown in the tables below. There was no change in the level of high molecular weight species in any formulation after reconstituted and placed at 5° or 25° C. for 1 week. The level of low molecular weight species appeared to increase slightly upon storage at 25° C. after reconstitution and essentially remained the same after 5° C. reconstituted storage. These results show that all formulations would remain stable if reconstituted and then placed at 5° or 25° C. for 1 week.

Samples stored at 5° C. for reconstituted stability were also analyzed by cation exchange chromatography. The results for all three formulations showed a slight increase in more acidic species after 1 week, regardless of their storage temperature prior to reconstitution. The samples that had been stored at 5° C. prior to reconstitution also showed a slight shift towards a more basic species after 1 week of storage. The samples stored at 40° C. prior to reconstitution showed less of a shift towards more basic species after 1 week of storage at 5° C. These changes are very minor and may be more indicative of assay variability than actual changes to the protein.

### Conclusions

The foregoing Study 4 has shown that a 4 mL volume can be successfully lyophilized in a 5 mL Kimble vial and have acceptable cake structure.

The reconstitution volumes determined to give the target protein concentration of 150 mg/mL, was 0.85 mL for 4089-1L (40 mg/mL pre-lyophilized concentration), 1.0 mL for 4089-1M (45 mg/mL pre-lyophilized concentration) and 1.1 mL for 4089-1N (50 mg/mL pre-lyophilized concentration). 4089-1N was the only formulation able to deliver a 1 mL dose, showing that 1.1 mL of water was the minimum reconstitution volume required. These results can be used to guide the final presentation developed with regards to fill volume, total solids and reconstitution volume. In general, samples with a lower initial protein concentration and higher sucrose to protein ratio showed shorter reconstitution times and slightly better stability with respect to loss of monomer. Based on fill volume and reconstitution volume, this indicates that a lower final protein concentration may give a more desirable product.

The reconstitution stability showed that all formulations were stable upon storage at 5° and 25° C. for 1 week, with no loss of monomer.

All three pre-lyophilized formulations remained stable at 5° C. over 6 months. However, upon storage at 40° C., all formulations showed a trend toward increasing amounts of low molecular weight species. The formation of low molecular weight species appears to be an important degradation pathway in the liquid samples given its fast rate of formation.

The lyophilized formulations also proved to be stable up to 6 months at 5° C. Upon storage at 40° C., there was a definite trend toward increasing amounts of high molecular weight species, while the amount of low molecular weight species remained the same. This shows that the formation of high molecular weight species is a more important degradation pathway in lyophilized samples. Additional optimization of the residual moisture levels and sucrose to protein ratios should improve the stability.

TABLE 27
Pre-Lyo
40 mg/mL Natalizumab, 24 mg/mL Sucrose, 6 mM Histidine and 0.02% PS-80
Prepared:
Testing Start Date: 0.5 mL fill

| | | | | | | | | | | % |
|---|---|---|---|---|---|---|---|---|---|---|
| Time | | | | | | | | | SEC Ref | Acidic % |
| Point/ | | | Conc. | Turbidity | % | % | % | % | Std (Avg. | Main % |
| Temp. | Vial | Appearance/pH | (mg/mL) | A360mn | Monomer | Dimer | Aggregate | LMW | area count) | Basic |
| 0 | 1 | sl opal, colorless/ | 40.9 | 0.036 | 98.03 | 1.23 | 0.63 | 0.11 | REF025 | 20.9 |
| | | 6.07 | | | | | | | (33849) | 67.9 |
| | | | | | | | | | | 11.2 |
| 2 wk, | 1 | very sl opal, | 42.2 | 0.047 | 97.75 | 1.16 | 0.45 | 0.64 | REF025 | 39.7 |
| 40 C. | | colorless | | | | | | | (33582) | 51.5 |
| | | | | | | | | | | 8.8 |
| | 2 | very sl opal, | 40.4 | 0.051 | 97.75 | 1.15 | 0.44 | 0.66 | | 42.1 |
| | | colorless | | | | | | | | 49.4 |
| | | | | | | | | | | 8.5 |
| 4 wk, | 1 | very sl opal, | 43.0 | 0.070 | 93.84 | 1.19 | 0.45 | 4.52 | REF025 | N/A |
| 40 C. | | colorless | | | | | | | (33610) | |
| | 2 | very sl opal, | 43.4 | 0.072 | 94.19 | 1.21 | 0.47 | 4.12 | | |
| | | colorless | | | | | | | | |
| 8 wk, | 1 | very sl opal, | 42.3 | 0.091 | 90.31 | 1.36 | 0.82 | 7.51 | REF025 | N/A |
| 40 C. | | colorless | | | | | | | (33838) | |
| | 2 | very sl opal, | 41.4 | 0.091 | 90.33 | 1.36 | 0.91 | 7.40 | | |
| | | colorless | | | | | | | | |
| 6 mo, | 1 | very sl opal, | 41.1 | 0.057 | 98.11 | 1.25 | 0.47 | 0.18 | REF025 | 25.9 |
| 5 C. | | colorless | | | | | | | (33851) | 60.5 |
| | | | | | | | | | | 13.6 |
| | 2 | very sl opal, | 40.9 | 0.058 | 98.10 | 1.25 | 0.47 | 0.18 | | 26.1 |
| | | colorless | | | | | | | | 61.2 |
| | | | | | | | | | | 12.8 |

TABLE 28
Pre-Lyo
40 mg/mL Natalizumab, 24 mg/mL Sucrose, 6 mM Histidine and 0.02% PS-80
Prepared:
Testing Start Date: 0.5 mL fill

| | | | | | | | | | | % |
|---|---|---|---|---|---|---|---|---|---|---|
| Time | | | | | | | | | SEC Ref Std | Acidic % |
| Point/ | | | Conc. | Turbidity | % | % | % | % | (Avg. area | Main % |
| Temp. | Vial | Appearance/pH | (mg/mL) | A360 mn | Monomer | Dimer | Aggregate | LMW | count) | Basic |
| 0 | 1 | sl opal, colorless/ | 48.3 | 0.041 | 98.02 | 1.23 | 0.63 | 0.11 | REF025 | 22.6 |
| | | 6.07 | | | | | | | (33849) | 64.5 |
| | | | | | | | | | | 12.9 |
| 2 wk, | 1 | very sl opal, | 48.6 | 0.058 | 97.66 | 1.16 | 0.44 | 0.74 | REF025 | 41.5 |
| 40 C. | | colorless | | | | | | | (33582) | 44.2 |
| | | | | | | | | | | 14.3 |
| | 2 | very sl opal, | 45.5 | 0.063 | 97.67 | 1.18 | 0.43 | 0.73 | | 41.3 |
| | | colorless | | | | | | | | 50.1 |
| | | | | | | | | | | 8.5 |
| 4 wk, | 1 | very sl opal, | 49.0 | 0.081 | 93.41 | 1.22 | 0.48 | 4.89 | REF025 | N/A |
| 40 C. | | colorless | | | | | | | (33610) | |
| | 2 | very sl opal, | 49.2 | 0.079 | 93.64 | 1.22 | 0.47 | 4.67 | | |
| | | colorless | | | | | | | | |
| 8 wk, | 1 | very sl opal, | 48.2 | 0.108 | 90.14 | 1.43 | 1.11 | 7.32 | REF025 | N/A |
| 40 C. | | colorless | | | | | | | (33838) | |
| | 2 | very sl opal, | 50.0 | 0.161 | 90.46 | 1.43 | 1.11 | 7.00 | | |
| | | colorless | | | | | | | | |
| 6 mo, | 1 | very sl opal, | 46.1 | 0.066 | 98.09 | 1.25 | 0.48 | 0.18 | REF025 | 26.2 |
| 5 C. | | colorless | | | | | | | (33851) | 60.8 |
| | | | | | | | | | | 13.0 |
| | 2 | very sl opal, | 46.0 | 0.068 | 98.11 | 1.25 | 0.47 | 0.16 | | 26.3 |
| | | colorless | | | | | | | | 60.6 |
| | | | | | | | | | | 13.1 |

TABLE 29
Pre-Lyo
50 mg/mL Natalizumab, 24 mg/mL Sucrose, 6 mM Histidine and 0.02% PS-80
Prepared:
Testing Start Date: 0.5 mL fill

| | | | | | | | | | | % |
|---|---|---|---|---|---|---|---|---|---|---|
| Time | | | | | | | | | SEC Ref Std | Acidic % |
| Point/ | | | Conc. | Turbidity | % | % | % | % | (Avg. area | Main % |
| Temp. | Vial | Appearance/pH | (mg/mL) | A360 mn | Monomer | Dimer | Aggregate | LMW | count) | Basic |
| 0 | 1 | sl opal, colorless/ | 52.6 | 0.053 | 98.01 | 1.24 | 0.63 | 0.12 | REF025 | 21.7 |
| | | 6.08 | | | | | | | (33849) | 67.6 |
| | | | | | | | | | | 10.8 |
| 2 wk, | 1 | very sl opal, | 55.9 | 0.062 | 97.61 | 1.17 | 0.43 | 0.79 | REF025 | 44.3 |
| 40 C. | | colorless | | | | | | | (33582) | 47.9 |
| | | | | | | | | | | 7.8 |
| | 2 | very sl opal, | 54.5 | 0.062 | 97.60 | 1.17 | 0.43 | 0.80 | | 44.0 |
| | | colorless | | | | | | | | 48.4 |
| | | | | | | | | | | 7.5 |
| 4 wk, | 1 | very sl opal, | 55.8 | 0.087 | 93.60 | 1.25 | 0.55 | 4.60 | REF025 | N/A |
| 40 C. | | colorless | | | | | | | (33610) | |
| | 2 | very sl opal, | 55.9 | 0.084 | 93.04 | 1.22 | 0.48 | 5.27 | | |
| | | colorless | | | | | | | | |
| 8 wk, | 1 | very sl opal, | 55.3 | 0.118 | 89.19 | 1.42 | 1.13 | 8.26 | REF025 | N/A |
| 40 C. | | colorless | | | | | | | (33838) | |
| | 2 | very sl opal, | 53.9 | 0.128 | 88.61 | 1.40 | 1.14 | 8.86 | | |
| | | colorless | | | | | | | | |
| 6 mo, | 1 | very sl opal, | 51.9 | 0.072 | 98.08 | 1.26 | 0.48 | 0.19 | REF025 | 26.5 |
| 5 C. | | colorless | | | | | | | (33851) | 60.6 |
| | | | | | | | | | | 13.0 |
| | 2 | very sl opal, | 52.3 | 0.067 | 98.08 | 1.25 | 0.48 | 0.19 | | 26.6 |
| | | colorless | | | | | | | | 61.1 |
| | | | | | | | | | | 12.4 |

TABLE 30
Lyophilized
150 mg/mL Natalizumab, 90 mg/mL Sucrose, 24 mM Histidine and 0.08% PS-80, pH 6
Reconstitute with 0.85 mL DI water to get 0.75-0.85 mL total volume
Prepared:
Lyophilization Complete:
Testing Start Date:

| | | | | | | | | | | | SEC | % |
|---|---|---|---|---|---|---|---|---|---|---|---|---|
| | | | | | | | | | | | Ref Std | Acidic |
| Time | | | Recon | | | | | | | | (Avg. | % |
| Point/ | | | Time | KF % | Conc. | Turbidity | % | % | % | % | area | Main % |
| Temp. | Vial | Appearance | (min.) | Moisture | (mg/mL) | A360 nm | Monomer | Dimer | Aggregate | LMW | count) | Basic |
| 0 | 1 | sl opal, | 2.2 | 4.5 | 164.8 | 0.102 | 97.87 | 1.38 | 0.61 | 0.14 | REF025 | 21.1 |
| | | colorless | | | | | | | | | (33709) | 65.1 |
| | | | | | | | | | | | | 13.8/ |
| | 2 | sl opal, | 2.2 | 4.6 | 164.5 | 0.099 | 97.87 | 1.38 | 0.61 | 0.13 | | 21.4 |
| | | colorless | | | | | | | | | | 65.1 |
| | | | | | | | | | | | | 13.5 |
| | 3 | sl opal, | 3.4 | 4.3 | 153.7 | 0.103 | 97.89 | 1.37 | 0.61 | 0.13 | | 21.4 |
| | | colorless | | | | | | | | | | 65.0 |
| | | | | | | | | | | | | 13.6 |
| 2 wk, | 1 | sl opal, | 4.4 | N/A | 161.9 | 0.109 | 96.99 | 2.19 | 0.60 | 0.21 | REF025 | 19.7 |
| 40 C. | | colorless | | | | | | | | | (33582) | 63.5 |
| | | | | | | | | | | | | 16.9 |
| | 2 | sl opal, | 3.7 | | 157.2 | 0.111 | 97.00 | 2.18 | 0.60 | 0.23 | | 20.1 |
| | | colorless | | | | | | | | | | 68.3 |
| | | | | | | | | | | | | 11.6 |
| 4 wk, | 1 | sl opal, | 3.9 | N/A | 159.2 | 0.136 | 96.50 | 2.63 | 0.63 | 0.24 | REF025 | 20.2 |
| 40 C. | | colorless | | | | | | | | | (33610) | 67.4 |
| | | | | | | | | | | | | 12.5 |
| | 2 | sl opal, | 5.7 | | 159.1 | 0.135 | 96.56 | 2.60 | 0.63 | 0.20 | | 20.2 |
| | | colorless | | | | | | | | | | 67.2 |
| | | | | | | | | | | | | 12.5 |
| 8 wk, | 1 | sl opal, | 3.8 | N/A | 166.4 | 0.156 | 95.76 | 3.39 | 0.69 | 0.16 | REF025 | 23.9 |
| 40 C. | | colorless | | | | | | | | | (33838) | 58.1 |
| | | | | | | | | | | | | 18.0 |
| | 2 | sl opal, | 2.9 | | 164.1 | 0.155 | 95.78 | 3.37 | 0.67 | 0.17 | | 21.8 |
| | | colorless | | | | | | | | | | 60.0 |
| | | | | | | | | | | | | 18.2 |
| 6 mo, | 1 | sl opal, | 9.3 | N/A | 153.7 | 0.113 | 97.83 | 1.47 | 0.59 | 0.15 | REF025 | 21.0 |
| 5 C. | | colorless | | | | | | | | | (33851) | 65.8 |
| | | | | | | | | | | | | 13.2 |
| | 2 | sl opal, | 7.4 | | 152.3 | 0.113 | 97.83 | 1.47 | 0.59 | 0.16 | | 21.0 |
| | | colorless | | | | | | | | | | 65.7 |
| | | | | | | | | | | | | 13.2 |

TABLE 31
RECON STABILITY: SAMPLES STORED 4 WEEKS AT 40 C. PRIOR TO RECONSTITUTION.

| | | | | | | | | | | | SEC Ref | % |
|---|---|---|---|---|---|---|---|---|---|---|---|---|
| Storage | | | | Recon | | | | | | | Std (Avg. | Acidic % |
| After | Time | | | Time | Conc. | Turbidity | % | % | % | % | area | Main % |
| Recon | point | Vial | Appearance | (min.) | (mg/mL) | A360 nm | Monomer | Dimer | Aggregate | LMW | count) | Basic |
| 5 C. | Initial | 1 | sl opal, | 3.9 | 159.2 | 0.136 | 96.50 | 2.63 | 0.63 | 0.24 | REF025 | 20.2 |
| | | | colorless | | | | | | | | (33610) | 67.4 |
| | | | | | | | | | | | | 12.5 |
| | | 2 | sl opal, | 5.7 | 159.1 | 0.135 | 96.56 | 2.60 | 0.63 | 0.20 | | 20.2 |
| | | | colorless | | | | | | | | | 67.2 |
| | | | | | | | | | | | | 12.5 |
| | 1 wk | 1 | sl opal, | N/A | 154.6 | 0.132 | 96.57 | 2.60 | 0.60 | 0.23 | REF025 | 23.0 |
| | | | colorless | | | | | | | | (33604) | 66.4 |
| | | | | | | | | | | | | 10.6 |
| | | 2 | sl opal, | | 159.2 | 0.132 | 96.61 | 2.56 | 0.60 | 0.24 | | 22.4 |
| | | | colorless | | | | | | | | | 69.0 |
| | | | | | | | | | | | | 8.6 |
| 25 C. | Initial | 3 | sl opal, | 4.9 | N/A | 0.137 | 96.55 | 2.61 | 0.63 | 0.20 | REF025 | N/A |
| | | | colorless | | | | | | | | (33610) | |
| | | 4 | sl opal, | 4.4 | | 0.135 | 96.52 | 2.64 | 0.63 | 0.21 | | |
| | | | colorless | | | | | | | | | |
| | 1 wk | 3 | sl opal, | N/A | 150.1 | 0.150 | 96.72 | 2.44 | 0.56 | 0.29 | REF025 | 20.6 |
| | | | colorless, | | | | | | | | (33604) | 69.6 |
| | | | “fluffy” | | | | | | | | | 9.8 |
| | | | white | | | | | | | | | |
| | | | particulates | | | | | | | | | |
| | | 4 | sl opal, | | 153.6 | 0.125 | 96.70 | 2.47 | 0.56 | 0.27 | | 20.3 |
| | | | colorless | | | | | | | | | 70.2 |
| | | | | | | | | | | | | 9.5 |

TABLE 32
RECON STABILITY: SAMPLES STORED 4 WEEKS AT 5 C. PRIOR TO RECONSTITUTION.

| Storage | | | | Recon | | | | | | | SEC Ref | % Acidic |
|---|---|---|---|---|---|---|---|---|---|---|---|---|
| After | Time | | | Time | Conc. | Turbidity | % | % | % | % | Std (Avg. | % Main |
| Recon | point | Vial | Appearance | (min.) | (mg/mL) | A360 nm | Monomer | Dimer | Aggregate | LMW | area count) | % Basic |
| 5 C. | Initial | 1 | sl opal, | 6.0 | 160.9 | 0.106 | 97.78 | 1.40 | 0.63 | 0.19 | REF025 | 20.2 |
| | | | colorless | | | | | | | | (33620) | 69.2 |
| | | | | | | | | | | | | 10.7 |
| | | 2 | sl opal, | 3.8 | 161.7 | 0.106 | 97.77 | 1.39 | 0.63 | 0.21 | | 19.8 |
| | | | colorless | | | | | | | | | 69.5 |
| | | | | | | | | | | | | 10.7 |
| | 1 wk | 1 | sl opal, | N/A | 150.6 | 0.107 | 97.80 | 1.39 | 0.60 | 0.22 | REF025 | 22.6 |
| | | | colorless | | | | | | | | (33604) | 60.2 |
| | | | | | | | | | | | | 17.2 |
| | | 2 | sl opal, | | 157.6 | 0.109 | 97.79 | 1.39 | 0.60 | 0.23 | | 23.5 |
| | | | colorless | | | | | | | | | 64.4 |
| | | | | | | | | | | | | 12.1 |
| 25 C. | Initial | 3 | sl opal, | 4.9 | N/A | N/A | N/A | N/A | N/A | N/A | N/A | N/A |
| | | | colorless | | | | | | | | | |
| | | 4 | sl opal, | 7.4 | | | | | | | | |
| | | | colorless | | | | | | | | | |
| | | 3 | sl opal, | N/A | 162.8 | 0.109 | 97.83 | 1.36 | 0.55 | 0.25 | REF025 | 20.8 |
| | | | colorless | | | | | | | | (33604) | 60.5 |
| | | | | | | | | | | | | 18.7 |
| | 1 wk | 4 | sl opal, | | 153.9 | 0.112 | 97.80 | 1.38 | 0.55 | 0.27 | | 20.1 |
| | | | colorless | | | | | | | | | 62.0 |
| | | | | | | | | | | | | 18.0 |

| Lyophilized | | | | | | | | | Prepared: | | | |
|---|---|---|---|---|---|---|---|---|---|---|---|---|
| 150 mg/mL Natalizumab, 90 mg/mL Sucrose, 24 mM Histidine and 0.08% PS-80, pH 6 | | | | | | | | | Lyophilization Complete: | | | |
| Reconstitute with 1.0 mL DI water to get 0.8-0.9 mL total volume | | | | | | | | | Testing Start Date: | | | |
| Time | | | Recon | KF | | | | | | | SEC Ref | % Acidic |
| Point/ | | | Time | % | Conc. | Turbidity | % | % | % | % | Std (Avg. | % Main |
| Temp. | Vial | Appearance | (min.) | Moisture | (mg/mL) | A360 nm | Monomer | Dimer | Aggregate | LMW | area count) | % Basic |
| 0 | 1 | sl opal, | 2.6 | 4.8 | 162.5 | 0.099 | 97.85 | 1.39 | 0.61 | 0.14 | REF025 | 21.0 |
| | | colorless | | | | | | | | | (33709) | 65.0 |
| | | | | | | | | | | | | 14.0 |
| | 2 | sl opal, | 6.5 | 5.3 | 152.0 | 0.102 | 97.86 | 1.39 | 0.61 | 0.14 | | 21.1 |
| | | colorless | | | | | | | | | | 65.1 |
| | | | | | | | | | | | | 13.8 |
| | 3 | sl opal, | 3.7 | 5.6 | 159.7 | 0.104 | 97.85 | 1.40 | 0.61 | 0.14 | | 21.7 |
| | | colorless | | | | | | | | | | 68.1 |
| | | | | | | | | | | | | 10.2 |
| 2 wk, | 1 | sl opal, | 4.7 | N/A | 151.4 | 0.109 | 96.76 | 2.43 | 0.60 | 0.21 | REF025 | 19.9 |
| 40 C. | | colorless | | | | | | | | | (33582) | 67.9 |
| | | | | | | | | | | | | 12.2 |
| | 2 | sl opal, | 4.8 | | 155.2 | 0.112 | 96.74 | 2.41 | 0.60 | 0.24 | | 20.1 |
| | | colorless | | | | | | | | | | 67.7 |
| | | | | | | | | | | | | 12.2 |
| 4 wk, | 1 | sl opal, | 5.4 | N/A | 160.8 | 0.137 | 96.12 | 3.03 | 0.66 | 0.19 | REF025 | 19.8 |
| 40 C. | | colorless | | | | | | | | | (33610) | 63.2 |
| | | | | | | | | | | | | 17.0 |
| | 2 | sl opal, | 3.8 | | 159.6 | 0.132 | 96.22 | 2.94 | 0.66 | 0.19 | | 20.2 |
| | | colorless | | | | | | | | | | 67.4 |
| | | | | | | | | | | | | 12.4 |
| 8 wk, | 1 | sl opal, | 5.5 | N/A | 160.7 | 0.154 | 95.19 | 3.92 | 0.73 | 0.16 | REF025 | 23.7 |
| 40 C. | | colorless | | | | | | | | | (33838) | 57.2 |
| | | | | | | | | | | | | 19.1 |
| | 2 | sl opal, | 7.3 | | 160.2 | 0.155 | 95.18 | 3.91 | 0.73 | 0.17 | | 21.7 |
| | | colorless | | | | | | | | | | 58.9 |
| | | | | | | | | | | | | 19.4 |
| 6 mo, | 1 | sl opal, | 5.8 | N/A | 146.3 | 0.111 | 97.78 | 1.52 | 0.60 | 0.15 | REF025 | 21.1 |
| 5 C. | | colorless | | | | | | | | | (33851) | 65.6 |
| | | | | | | | | | | | | 13.3 |
| | 2 | sl opal, | 7.0 | | 147.3 | 0.116 | 97.77 | 1.53 | 0.59 | 0.16 | | 21.1 |
| | | colorless | | | | | | | | | | 65.0 |
| | | | | | | | | | | | | 14.0 |

TABLE 33
RECON STABILITY: SAMPLES STORED 4 WEEKS AT 40 C. PRIOR TO RECONSTITUTION

| | | | | | | | | | | | SEC Ref | |
|---|---|---|---|---|---|---|---|---|---|---|---|---|
| | | | | | | | | | | | Std | % |
| Storage | | | | Recon | | | | | | | (Avg. | Acidic % |
| After | Time | | | Time | Conc. | Turbidity | % | % | % | % | area | Main % |
| Recon | point | Vial | Appearance | (min.) | (mg/mL) | A360 nm | Monomer | Dimer | Aggregate | LMW | count) | Basic |
| 5 C. | Initial | 1 | sl opal, | 5.4 | 160.8 | 0.137 | 96.12 | 3.03 | 0.66 | 0.19 | REF025 | 19.8 |
| | | | colorless | | | | | | | | (33610) | 63.2 |
| | | | | | | | | | | | | 17.0 |
| | | 2 | sl opal, | 3.8 | 159.6 | 0.132 | 96.22 | 2.94 | 0.66 | 0.19 | | 20.2 |
| | | | colorless | | | | | | | | | 67.4 |
| | | | | | | | | | | | | 12.4 |
| | 1 wk | 1 | sl opal, | N/A | 153.7 | 0.131 | 96.16 | 2.97 | 0.62 | 0.24 | REF025 | 22.5 |
| | | | colorless | | | | | | | | (33604) | 62.7 |
| | | | | | | | | | | | | 14.8 |
| | | 2 | sl opal, | | 152.2 | 0.131 | 96.27 | 2.88 | 0.62 | 0.24 | | 22.7 |
| | | | colorless | | | | | | | | | 68.3 |
| | | | | | | | | | | | | 9.0 |
| 25 C. | Initial | 3 | sl opal, | 4.0 | N/A | 0.136 | 96.11 | 3.02 | 0.67 | 0.19 | REF025 | N/A |
| | | | colorless | | | | | | | | (33610) | |
| | | 4 | sl opal, | 4.1 | | 0.138 | 96.17 | 2.96 | 0.66 | 0.21 | | |
| | | | colorless | | | | | | | | | |
| | 1 wk | 3 | sl opal, | N/A | 148.9 | 0.132 | 96.30 | 2.84 | 0.58 | 0.2 | REF025 | 20.1 |
| | | | colorless, | | | | | | | | (33604) | 70.5 |
| | | | “fluffy” | | | | | | | | | 9.4 |
| | | | white | | | | | | | | | |
| | | | particulates | | | | | | | | | |
| | | 4 | sl opal, | | 147.5 | 0.130 | 96.37 | 2.78 | 0.57 | 0.28 | | 19.8 |
| | | | colorless | | | | | | | | | 70.4 |
| | | | | | | | | | | | | 9.8 |

TABLE 34
RECON STABILITY: SAMPLES STORED 4 WEEKS AT 5 C. PRIOR TO RECONSTITUTION.

| | | | | | | | | | | | SEC | |
|---|---|---|---|---|---|---|---|---|---|---|---|---|
| | | | | | | | | | | | Ref Std | % |
| Storage | | | | Recon | | | | | | | (Avg. | Acidic % |
| After | Time | | | Time | Conc. | Turbidity | % | % | % | % | area | Main % |
| Recon | point | Vial | Appearance | (min.) | (mg/mL) | A360 nm | Monomer | Dimer | Aggregate | LMW | count) | Basic |
| 5 C. | Initial | 1 | sl opal, | 4.5 | 150.1 | 0.108 | 97.79 | 1.40 | 0.62 | 0.18 | REF025 | 20.3 |
| | | | colorless | | | | | | | | (33620) | 68.8 |
| | | | | | | | | | | | | 10.9 |
| | | 2 | sl opal, | 4.8 | 149.2 | 0.106 | 97.78 | 1.41 | 0.62 | 0.19 | | 19.9 |
| | | | colorless | | | | | | | | | 70.4 |
| | | | | | | | | | | | | 9.7 |
| | 1 wk | 1 | sl opal, | N/A | 145.2 | 0.112 | 97.80 | 1.40 | 0.59 | 0.21 | REF025 | 23.8 |
| | | | colorless | | | | | | | | (33604) | 59.6 |
| | | | | | | | | | | | | 16.6 |
| | | 2 | sl opal, | | 145.8 | 0.111 | 97.79 | 1.40 | 0.59 | 0.22 | | 22.6 |
| | | | colorless | | | | | | | | | 60.8 |
| | | | | | | | | | | | | 16.6 |
| 25 C. | Initial | 3 | sl opal, | 3.3 | N/A | N/A | N/A | N/A | N/A | N/A | N/A | N/A |
| | | | colorless | | | | | | | | | |
| | | 4 | sl opal, | 3.1 | | | | | | | | |
| | | | colorless | | | | | | | | | |
| | 1 wk | 3 | sl opal, | N/A | 141.8 | 0.112 | 97.84 | 1.37 | 0.55 | 0.25 | REF025 | 20.8 |
| | | | colorless | | | | | | | | (33604) | 66.9 |
| | | | | | | | | | | | | 12.3 |
| | | 4 | sl opal, | | 140.0 | 0.111 | 97.84 | 1.37 | 0.54 | 0.25 | | 20.2 |
| | | | colorless | | | | | | | | | 61.8 |
| | | | | | | | | | | | | 18.0 |

TABLE 35
Lyophilized
150 mg/mL Natalizumab, 90 mg/mL Sucrose, 24 mM Histidine and 0.08% PS-80,
pH 6 Reconstitute with 1.1 mL DI water to get 1.0 mL total volume
Prepared:
Lyophilization Complete:
Testing Start Date:

| | | | | | | | | | | | SEC | % |
|---|---|---|---|---|---|---|---|---|---|---|---|---|
| | | | | | | | | | | | Ref Std | Acidic |
| Time | | | Recon | | | | | | | | (Avg. | % |
| Point/ | | | Time | KF % | Conc. | Turbidity | % | % | % | % | area | Main % |
| Temp. | Vial | Appearance | (min.) | Moisture | (mg/mL) | A360 nm | Monomer | Dimer | Aggregate | LMW | count) | Basic |
| 0 | 1 | sl opal, | 6.6 | 4.7 | 161.3 | 0.099 | 97.84 | 1.41 | 0.62 | 0.14 | REF025 | 21.3 |
| | | colorless | | | | | | | | | (33709) | 64.8 |
| | | | | | | | | | | | | 13.9 |
| | 2 | sl opal, | 5.8 | 4.7 | 168.6 | 0.100 | 97.84 | 1.41 | 0.62 | 0.14 | | 21.3 |
| | | colorless | | | | | | | | | | 64.7 |
| | | | | | | | | | | | | 14.0 |
| | 3 | sl opal, | 3.3 | 5.3 | 170.3 | 0.099 | 97.84 | 1.41 | 0.61 | 0.14 | | 21.6 |
| | | colorless | | | | | | | | | | 64.8 |
| | | | | | | | | | | | | 13.6 |
| 2 wk, | 1 | sl opal, | 11.6 | N/A | 164.0 | 0.116 | 96.56 | 2.62 | 0.62 | 0.21 | REF025 | 19.8 |
| 40 C. | | colorless | | | | | | | | | (33582) | 68.1 |
| | | | | | | | | | | | | 12.1 |
| | 2 | sl opal, | 6.9 | | 159.3 | 0.115 | 96.54 | 2.63 | 0.62 | 0.21 | | 20.0 |
| | | colorless | | | | | | | | | | 63.7 |
| | | | | | | | | | | | | 16.2 |
| 4 wk, | 1 | sl opal, | 5.6 | N/A | 163.3 | 0.135 | 95.87 | 3.25 | 0.69 | 0.19 | REF025 | 20.8 |
| 40 C. | | colorless | | | | | | | | | (33610) | 62.2 |
| | | | | | | | | | | | | 17.0 |
| | 2 | sl opal, | 7.2 | | 159.6 | 0.135 | 95.90 | 3.21 | 0.69 | 0.20 | | 20.1 |
| | | colorless | | | | | | | | | | 62.5 |
| | | | | | | | | | | | | 17.5 |
| 8 wk, | 1 | sl opal, | 7.9 | N/A | 162.7 | 0.157 | 94.79 | 4.26 | 0.78 | 0.17 | REF025 | 23.8 |
| 40 C. | | colorless | | | | | | | | | (33838) | 56.9 |
| | | | | | | | | | | | | 19.2 |
| | 2 | sl opal, | 16.1 | | 166.5 | 0.155 | 94.79 | 4.25 | 0.78 | 0.18 | | 23.7 |
| | | colorless | | | | | | | | | | 56.8 |
| | | | | | | | | | | | | 19.5 |
| 6 mo, | 1 | sl opal, | 6.0 | N/A | 149.1 | 0.115 | 97.75 | 1.56 | 0.60 | 0.15 | REF025 | 20.9 |
| 5 C. | | colorless | | | | | | | | | (33851) | 65.7 |
| | | | | | | | | | | | | 13.3 |
| | 2 | sl opal, | 9.7 | | 149.1 | 0.114 | 97.76 | 1.55 | 0.60 | 0.15 | | 21.1 |
| | | colorless | | | | | | | | | | 64.9 |
| | | | | | | | | | | | | 14.0 |

TABLE 36
RECON STABILITY: SAMPLES STORED 4 WEEKS AT 40 C. PRIOR TO RECONSTITUTION.

| | | | | | | | | | | | SEC Ref | |
|---|---|---|---|---|---|---|---|---|---|---|---|---|
| | | | | | | | | | | | Std | % |
| Storage | | | | Recon | | | | | | | (Avg. | Acidic % |
| After | Time | | | Time | Conc. | Turbidity | % | % | % | % | area | Main % |
| Recon | point | Vial | Appearance | (min.) | (mg/mL) | A360 nm | Monomer | Dimer | Aggregate | LMW | count) | Basic |
| 5 C. | Initial | 1 | sl opal, | 5.6 | 163.3 | 0.135 | 95.87 | 3.25 | 0.69 | 0.19 | REF025 | 20.8 |
| | | | colorless | | | | | | | | (33610) | 62.2 |
| | | | | | | | | | | | | 17.0 |
| | | 2 | sl opal, | 7.2 | 159.6 | 0.135 | 95.90 | 3.21 | 0.69 | 0.20 | | 20.1 |
| | | | colorless | | | | | | | | | 62.5 |
| | | | | | | | | | | | | 17.5 |
| | 1 wk | 1 | sl opal, | N/A | 159.1 | 0.133 | 95.92 | 3.19 | 0.64 | 0.24 | REF025 | 22.8 |
| | | | colorless | | | | | | | | (33604) | 66.6 |
| | | | | | | | | | | | | 10.5 |
| | | 2 | sl opal, | | 155.9 | 0.132 | 95.97 | 3.16 | 0.64 | 0.23 | | 23.2 |
| | | | colorless | | | | | | | | | 67.2 |
| | | | | | | | | | | | | 9.6 |
| 25 C. | Initial | 3 | sl, opal, | 8.6 | N/A | 0.136 | 95.86 | 3.23 | 0.69 | 0.22 | REF025 | N/A |
| | | | colorless | | | | | | | | (33610) | |
| | | 4 | sl opal, | 6.7 | | 0.136 | 95.87 | 3.25 | 0.69 | 0.19 | | |
| | | | colorless | | | | | | | | | |
| | 1 wk | 3 | sl opal, | N/A | 150.5 | 0.131 | 96.06 | 3.05 | 0.59 | 0.31 | REF025 | 20.4 |
| | | | colorless | | | | | | | | (33604) | 70.0 |
| | | | | | | | | | | | | 9.6 |
| | | 4 | sl opal, | | 149.9 | 0.128 | 96.07 | 3.05 | 0.59 | 0.29 | | 20.1 |
| | | | colorless | | | | | | | | | 70.5 |
| | | | | | | | | | | | | 9.4 |

TABLE 37
RECON STABILITY: SAMPLES STORED 4 WEEKS AT 5 C. PRIOR TO RECONSTITUTION.

| | | | | | | | | | | | SEC Ref | |
|---|---|---|---|---|---|---|---|---|---|---|---|---|
| | | | | | | | | | | | Std | % |
| Storage | | | | Recon | | | | | | | (Avg. | Acidic % |
| After | Time | | | Time | Conc. | Turbidity | % | % | % | % | area | Main % |
| Recon | point | Vial | Appearance | (min.) | (mg/mL) | A360 nm | Monomer | Dimer | Aggregate | LMW | count) | Basic |
| 5 C. | Initial | 1 | sl opal, | 7.1 | 162.0 | 0.107 | 97.72 | 1.43 | 0.62 | 0.22 | REF025 | 20.3 |
| | | | colorless | | | | | | | | (33620) | 69.1 |
| | | | | | | | | | | | | 10.5 |
| | | 2 | sl opal, | 6.1 | 164.7 | 0.105 | 97.74 | 1.44 | 0.63 | 0.19 | | 20.4 |
| | | | colorless | | | | | | | | | 69.0 |
| | | | | | | | | | | | | 10.6 |
| | 1 wk | 1 | sl opal, | N/A | 162.3 | 0.114 | 97.75 | 1.42 | 0.59 | 0.23 | REF025 | 23.6 |
| | | | colorless | | | | | | | | (33604) | 59.7 |
| | | | | | | | | | | | | 16.7 |
| | | 2 | sl opal, | | 161.8 | 0.109 | 97.72 | 1.42 | 0.60 | 0.26 | | 22.8 |
| | | | colorless | | | | | | | | | 61.4 |
| | | | | | | | | | | | | 15.8 |
| 25 C. | Initial | 3 | sl opal, | 4.6 | N/A | N/A | N/A | N/A | N/A | N/A | N/A | N/A |
| | | | colorless | | | | | | | | | |
| | | 4 | sl opal, | 5.1 | | | | | | | | |
| | | | colorless | | | | | | | | | |
| | 1 wk | 3 | sl opal, | N/A | 155.0 | 0.111 | 97.80 | 1.40 | 0.55 | 0.26 | REF025 | 21.2 |
| | | | colorless | | | | | | | | (33604) | 61.9 |
| | | | | | | | | | | | | 16.9 |
| | | 4 | sl opal, | | 153.4 | 0.109 | 97.80 | 1.40 | 0.55 | 0.25 | | 20.4 |
| | | | colorless | | | | | | | | | 62.5 |
| | | | | | | | | | | | | 17.25 |

Natalizumab Reformulation Development

Pre-Lyophilization Amounts

| | Antegren | Sucrose | Histidine | PS-80 |
|---|---|---|---|---|
| Name | (mg/mL) | (mg/mL) | (mM) | (%) |
| A | 40 | 27.7 | 6 | 0.04 |
| B | 40 | 46.2 | 6 | 0.04 |
| C | 40 | 9.2 | 6 | 0.04 |

TABLE 38

Target Post-Lyophilization Amounts

| | Antegren | Sucrose | Histidine | PS-80 |
|---|---|---|---|---|
| Name | (mg/mL) | (mg/mL) | (mM) | (%) |
| A1 | 160 | 110.8 | 24 | 0.16 |
| A2 | 100 | 69.3 | 15 | 0.1 |
| B3 | 160 | 184.8 | 24 | 0.16 |
| B4 | 40 | 46.2 | 6 | 0.04 |
| AS | 100 | 69.3 | 15 | 0.1 |
| A6 | 40 | 27.7 | 6 | 0.04 |
| C7 | 100 | 23.0 | 15 | 0.1 |
| C8 | 40 | 9.2 | 6 | 0.04 |
| C9 | 160 | 36.8 | 24 | 0.16 |
| A10 | 100 | 69.3 | 15 | 0.1 |
| B11 | 100 | 115.5 | 15 | 0.1 |

### Example 4

The purpose of this study was to use the design of experiments to determine the effect of final protein concentration and the optimal protein to sucrose ratio for lyophilization of natalizumab to prevent and minimize aggregate formation.

### Experimental Design Parameters

The final protein concentrations from 40 mg/mL to 160 mg/mL were examined with a protein to sucrose ratio of 1:100 to 1:500. The polysorbate 80 level was held constant at an amount of 0.01% per 10 mg/mL protein, and the histidine will be held constant at 10 mM per 40 mg/mL protein. Starting protein concentration (pre-lyophilization) will be 40 mg/mL. Vials were filled at a 2 mL fill, and then reconstituted to the desired final protein concentration.

Short term stability was examined at 0, 2, 4 and 6 weeks at 40 degrees Celcius.

### Procedure:

1. Diafilter the current formulation against one of the following buffers. Then ultrafilter to a protein concentration of >40 mg/mL. Measure the protein concentration and dilute to 40 mg/mL.
   a. Formulation SA: 100:1 sucrose:protein ratio—final formulations 7, 8, 9
   b. Formulation SB: 300:1 sucrose:protein ratio—final formulations 1, 2, 5, 6, 10
   c. Formulation SC: 500:1 sucrose:protein ratio—final formulations 3, 4, 11
2. 20 vials of each formulation will be filled at 2 mL/vial. This allows 10 vials for stability at 40 deg and addition vials for Tg, moisture, FTIR analysis, osmolality, etc. Amounts needed:
   a. Formulation SA: 120 mL
   b. Formulation SB: 200 mL
   c. Formulation SC: 120 mL
3. Vials are lyophilized using a conservative cycle to decrease moisture and reconstituted to the desired protein concentration
   a. Formulations 4, 6, 8—reconstitute with 2 mL for a final concentration of 40 mg/mL
   b. Formulations 1, 2, 5, 6, 10—reconstitute with 0.8 mL to give a final concentration of 100 mg/mL
   c. Formulations 1, 3.9—reconstitute with 0.5 mL to give a final concentration of 160 mg/mL
4. Stability is examined at 2, 4 and 6 weeks at 40 deg. by turbidity, concentration, appearance, SEC. Additional assay may be run at time 0 and 6 weeks, such as IEX, non-reduced SDS-PAGE and oxidation.

TABLE 39
Formulation Number

| Final | Initial | final proten | protein:sucrose | Recon volume |
|---|---|---|---|---|
| 1 | A | 160 | 300 | 1 |
| 2 | A | 100 | 300 | 1.6 |
| 3 | B | 160 | 500 | 1 |
| 4 | B | 40 | 500 | 4 |
| 5 | A | 100 | 300 | 1.6 |
| 6 | A | 40 | 300 | 4 |
| 7 | C | 100 | 100 | 1.6 |
| 8 | C | 40 | 100 | 4 |
| 9 | C | 160 | 100 | 1 |
| 10 | A | 100 | 300 | 1.6 |
| 11 | B | 100 | 500 | 1.6 |

| | |
|---|---|
| Formulation A: 40 mg/mL protein, 6 mM | Used 5 formulations |
| histidine, 0.04% PS80, 27.7 mg/mL | |
| Formulation B: 40 mg/mL protein, 6 mM | Used for 3 formulations |
| histidine, 0.04% PS80, 46.2 mg/mL | |
| Formulation C: 40 mg/mL protein, 6 mM | Used for 3 formulations |
| histidine, 0.04% PS80, 9.2 mg/mL | |
| Fill each vial at 4 ml = 160 mg/vial | Reconstitute to desired |
| | volume as above |
| Total protein used = 4 mL * 40 mg/mL * | 350 vials |
| 11 formulations * 30 vials | |
| 52800 | |

| | | | |
|---|---|---|---|
| Form A | 600 mL | 80 vials | DF vs 3 It |
| Form B | 360 | 48 vials | DF vs 2 It |
| Form C | 360 | 48 vials | DF vs 2 It |

UF to approx ½ volume

TABLE 40
160 mg/mL Antegren, 110.8 mg/mL Sucrose, 24 mM Histidine,
0.16% PS-80
1 vial tested per time point.
Prepared:
Lyophilization Co Complete
Testing Start Date:
At T = 0 reconstituted with 1.0 mL., all other time points
reconstituted with 0.8 mL.
2-8 C.

| | | Recon | | | | | | | | SEC Total | % Acidic |
|---|---|---|---|---|---|---|---|---|---|---|---|
| Time | Appearance, | Time | KF % | Conc. | Turbidity | % | % | % | % | Peak Area | % Main |
| Point | pH | (min.) | Moisture | (mg/mL) | A360 nm | Monomer | Dimer | Aggregate | LMW | Count | % Basic |
| 0 | IV, | 3.8 | 3.4 | 126.9 | 0.126 | 99.50 | 0.50 | — | — | 33274 | 15.8 |
| | Colorless | | | | | | | | | | 66.7 |
| | | | | | | | | | | | 17.6 |
| 4 wk | III, | 4.5 | N/A | 150.3 | 0.101 | 99.27 | 0.55 | 0.10 | 0.07 | 39516 | 13.6 |
| | Colorless | | | | | | | | | | 71.2 |
| | | | | | | | | | | | 15.2 |
| 8 wk | IV, BY7 | 4.7 | N/A | 159.5 | 0.110 | 99.22 | 0.68 | — | 0.10 | 41343 | 14.9 |
| | 6.37 | | | | | | | | | | 70.5 |
| | | | | | | | | | | | 14.7 |
| 12 wk | 1V, BY7 | 6.3 | N/A | 141.4 | 0.108 | 99.21 | 0.71 | — | 0.08 | 40153 | 15.2 |
| | | | | | | | | | | | 70.5 |
| | | | | | | | | | | | 14.3 |

25 C.

| | | Recon | | | | | | | SEC Total | % Acidic |
|---|---|---|---|---|---|---|---|---|---|---|
| Time | Appearance, | Time | Conc. | Turbidity | % | % | % | % | Peak Area | % Main |
| Point | pH | (min.) | (mg/mL) | A360 nm | Monomer | Dimer | Aggregate | LMW | Count | % Basic |
| 4 wk | IV, | 4.7 | 151.7 | 0.111 | 98.89 | 1.03 | — | 0.07 | 39584 | 14.5 |
| | Colorless | | | | | | | | | 70.3 |
| | | | | | | | | | | 15.2 |
| 6 wk | 1V, BY7 | 2.8 | 152.4 | 0.132 | 98.79 | 1.13 | — | 0.08 | 39933 | 14.6 |
| | | | | | | | | | | 69.6 |
| | | | | | | | | | | 15.8 |
| 8 wk | IV, BY6 | 3.7 | 153.3 | 0.116 | 98.64 | 1.25 | — | 0.11 | 39826 | 15.0 |
| | 6.27 | | | | | | | | | 69.9 |
| | | | | | | | | | | 15.1 |
| 12 wk | IV, BY7 | 4.4 | 142.8 | 0.119 | 98.52 | 1.40 | — | 0.08 | 39447 | 15.2 |
| | | | | | | | | | | 68.9 |
| | | | | | | | | | | 15.9 |

40 C.

| | | Recon | | | | | | | SEC Total | % Acidic |
|---|---|---|---|---|---|---|---|---|---|---|
| Time | | Time | Conc. | Turbidity | % | % | % | % | Peak Area | % Main |
| Point | Appearance | (min.) | (mg/mL) | A360 nm | Monomer | Dimer | Aggregate | LMW | Count | % Basic |
| 2 wk | IV, | 4.1 | 1543 | 0.124 | 98.52 | 1.48 | — | — | 39011 | 15.1 |
| | Colorless | | | | | | | | | 66.4 |
| | | | | | | | | | | 185 |
| 4 wk | IV, BY7 | 4.1 | 130.7 | 0.113 | 97.81 | 2.10 | — | 0.09 | 34892 | 14.9 |
| | | | | | | | | | | 63.9 |
| | | | | | | | | | | 21.1 |
| 6 wk | IV, BY7 | 4.1 | 151.8 | 0.113 | 97.28 | 2.41 | 0.21 | 0.10 | 39626 | 14.8 |
| | | | | | | | | | | 65.9 |
| | | | | | | | | | | 19.3 |

TABLE 41
100 mg/mL Antegren, 69.3 mg/mL Sucrose, 15 mM Histidine, 0.1% PS-80
1 vial tested per time point.
Prepared:
Lyophilization Complete:
Testing Start Date:
At T = 0 reconstituted with 1.6 mL, all other time points
reconstituted with 1.3 mL.
2-8 C.

| | | Recon | | | | | | | | SEC Total | % Acidic |
|---|---|---|---|---|---|---|---|---|---|---|---|
| Time | Appearance, | Time | KF % | Conc. | Turbidity | % | % | % | % | Peak Area | % Main |
| Point | pH | (min.) | Moisture | (mg/mL) | A360 nm | Monomer | Dimer | Aggregate | LMW | Count | % Basic |
| 0 | IV, | 2.7 | 2.7 | 85.8 | 0.095 | 99.49 | 0.51 | — | — | 36736 | 15.7 |
| | Colorless | | | | | | | | | | 67.6 |
| | | | | | | | | | | | 16.8 |
| 4 wk | III, | 2.4 | N/A | 106.6 | 0.111 | 99.28 | 0.55 | 0.10 | 0.06 | 35806 | 14.0 |
| | Colorless | | | | | | | | | | 71.0 |
| | | | | | | | | | | | 15.0 |
| 8 wk | III, BY7 | 2.7 | N/A | 104.1 | 0.107 | 99.25 | 0.67 | — | 0.08 | 35381 | 15.3 |
| | 6.36 | | | | | | | | | | 70.7 |
| | | | | | | | | | | | 14.0 |
| 12 wk | III, BY7 | 4.2 | N/A | 97.1 | 0.107 | 99.21 | 0.72 | — | 0.07 | 35037 | 15.3 |
| | | | | | | | | | | | 70.4 |
| | | | | | | | | | | | 14.2 |

25 C.

| | | Recon | | | | | | | SEC Total | % Acidic |
|---|---|---|---|---|---|---|---|---|---|---|
| Time | Appearance, | Time | Conc. | Turbidity | % | % | % | % | Peak Area | % Main |
| Point | pH | (min.) | (mg/mL) | A360 nm | Monomer | Dimer | Aggregate | LMW | Count | % Basic |
| 4 wk | III, | 3.0 | 106.2 | 0.101 | 98.90 | 1.02 | — | 0.08 | 35825 | 14.7 |
| | Colorless | | | | | | | | | 70.8 |
| | | | | | | | | | | 14.5 |
| 6 wk | IV, BY7 | 4.8 | 103.8 | 0.106 | 98.81 | 1.12 | — | 0.07 | 35581 | 14.7 |
| | | | | | | | | | | 69.8 |
| | | | | | | | | | | 15.5 |
| 8 wk | III, BY7 | 4.2 | 102.3 | 0.101 | 98.67 | 1.23 | — | 0.09 | 35055 | 14.9 |
| | 6.34 | | | | | | | | | 69.3 |
| | | | | | | | | | | 15.8 |
| 12 wk | III, BY7 | 3.9 | 96.9 | 0.105 | 98.55 | 1.38 | — | 0.08 | 34771 | 15.4 |
| | | | | | | | | | | 68.9 |
| | | | | | | | | | | 15.6 |

40 C.

| | | Recoil | | | | | | | SEC Total | % Acidic |
|---|---|---|---|---|---|---|---|---|---|---|
| Time | | Time | Conc. | Turbidity | % | % | % | % | Peak Area | % Main |
| Point | Appearance | (min.) | (mg/mL) | A360 nm | Monomer | Dimer | Aggregate | LMW | Count | % Basic |
| 2 wk | III, | 3.3 | 105.0 | 0.106 | 98.53 | 1.47 | — | — | 35468 | 15.5 |
| | Colorless | | | | | | | | | 66.5 |
| | | | | | | | | | | 18.0 |
| 4 wk | III. BY7 | 2.8 | 104.7 | 0.110 | 97.76 | 2.16 | — | 0.08 | 35751 | 15.3 |
| | | | | | | | | | | 64.2 |
| | | | | | | | | | | 20.5 |
| 6 wk | IV, BY7 | 2.7 | 104.5 | 0.111 | 97.32 | 2.38 | 0.21 | 0.09 | 35788 | 15.1 |
| | | | | | | | | | | 66.5 |
| | | | | | | | | | | 18.4 |

TABLE 42
160 mg/mL Antegren, 184.8 mg/mL Sucrose, 24 mM Histidine,
0.16%. PS-80
1 vial tested per time point.
Prepared:
Lyophilization Complete:
Testing Start Date:
At T = 0 reconstituted with 1.0 mL, all other time points
reconstituted with 0.8 mL.
2-8 C.

| | | Recon | | | | | | | | SEC Total | % Acidic |
|---|---|---|---|---|---|---|---|---|---|---|---|
| Time | Appearance, | Time | KF % | Conc. | Turbidity | % | % | % | % | Peak Area | % Main |
| Point | pH | (min.) | Moisture | (mg/mL) | A360 nm | Monomer | Dimer | Aggregate | LMW | Count | % Basic |
| 0 | IV, | 3.4 | 2.3 | 121.4 | 0.112 | 99.55 | 0.45 | — | — | 31624 | 15.3 |
| | Colorless | | | | | | | | | | 67.2 |
| | | | | | | | | | | | 17.5 |
| 4 wk | III, | 5.4 | N/A | 148.1 | 0.124 | 99.37 | 0.46 | 0.10 | 0.07 | 37676 | 13.7 |
| | Colorless | | | | | | | | | | 71.3 |
| | | | | | | | | | | | 15.0 |
| 8 wk | III. BY7 | 4.1 | N/A | 145.3 | 0.105 | 99.34 | 0.57 | — | 0.10 | 37764 | 14.9 |
| | 6.36 | | | | | | | | | | 70.8 |
| | | | | | | | | | | | 14.3 |
| 12 wk | III, BY7 | 7.3 | N/A | 138.2 | 0.116 | 99.32 | 0.60 | — | 0.09 | 37653 | 15.1 |
| | | | | | | | | | | | 70.6 |
| | | | | | | | | | | | 14.3 |

25 C.

| | | Recon | | | | | | | SEC Total | % Acidic |
|---|---|---|---|---|---|---|---|---|---|---|
| Time | Appearance, | Time | Conc. | Turbidity | % | % | % | % | Peak Area | % Main |
| Point | pH | (min.) | (mg/mL) | A360 nm | Monomer | Dimer | Aggregate | LMW | Count | % Basic |
| 4 wk | III | 4.0 | 144.2 | 0.093 | 99.22 | 0.70 | — | 0.07 | 37807 | 14.7 |
| | Colorless | | | | | | | | | 70.9 |
| | | | | | | | | | | 14.4 |
| 6 wk | III, BY7 | 6.7 | 142.6 | 0.109 | 99.18 | 0.75 | — | 0.07 | 37627 | 14.7 |
| | | | | | | | | | | 70.6 |
| | | | | | | | | | | 14.7 |
| 8 wk | III, BY6 | 6.0 | 140.8 | 0.099 | 99.11 | 0.80 | — | 0.09 | 37563 | 15.2 |
| | 6.37 | | | | | | | | | 70.1 |
| | | | | | | | | | | 14.8 |
| 12 wk | IV, BY7 | 6.9 | 123.4 | 0.118 | 99.10 | 0.82 | — | 0.08 | 36746 | 15.4 |
| | | | | | | | | | | 69.9 |
| | | | | | | | | | | 14.6 |

40 C.

| | | Recon | | | | | | | SEC Total | % Acidic |
|---|---|---|---|---|---|---|---|---|---|---|
| Time | Appearance, | Time | Conc. | Turbidity | % | % | % | % | Peak Area | % Main |
| Point | pH | (min.) | (mg/mL) | A360 nm | Monomer | Dimer | Aggregate | LMW | Count | % Basic |
| 2 wk | IV. | 5.2 | 143.7 | 0.107 | 99.23 | 0.77 | — | — | 37128 | 15.6 |
| | Colorless | | | | | | | | | 66.6 |
| | | | | | | | | | | 17.9 |
| 4 wk | III, B Y7 | 4.0 | 141.1 | 0.102 | 98.84 | 1.08 | — | 0.08 | 37428 | 15.4 |
| | | | | | | | | | | 65.9 |
| | | | | | | | | | | 18.8 |
| 6 wk | III, BY7 | 3.5 | 141.0 | 0.114 | 98.65 | 1.27 | — | 0.08 | 37649 | 15.1 |
| | | | | | | | | | | 68.9 |
| | | | | | | | | | | 16.0 |

TABLE 43
40 mg/mL Antegren, 46.2 mg/mL Sucrose, 6 mM Histidine, 0.04% PS-80
1 vial tested per time point
Prepared:
Lyophilization Complete:
Testing Start Date:
At T = 0 reconstituted with 4.0 mL, all other time points
reconstituted with 3.9 mL.
2-8 C.
A360 nm

| | | Recon | | | | | | | | SEC Total | % Acidic |
|---|---|---|---|---|---|---|---|---|---|---|---|
| Time | Appearance | Time | KF % | Conc. | Turbidity | % | % | % | % | Peak Area | % Main |
| Point | pH. | (min.) | Moisture | (mg/mL) | A360 nm | Monomer | Dimer | Aggregate | LMW | Count | % Basic |
| 0 | III, Colorless | 1.9 | 2.5 | 36.8 | 0.054 | 99.56 | 0.44 | — | — | 33960 | 15.9 |
| | | | | | | | | | | | 67.5 |
| | | | | | | | | | | | 16.6 |
| 4 wk | II, Colorless | 2.7 | N/A | 38.0 | 0.041 | 99.35 | 0.46 | 0.13 | 0.06 | 34758 | 14.0 |
| | | | | | | | | | | | 71.1 |
| | | | | | | | | | | | 14.9 |
| 8 wk | II, BY7 | 3.9 | N/A | 38.2 | 0.046 | 99.34 | 0.58 | — | 0.07 | 35571 | 15.2 |
| | 6.37 | | | | | | | | | | 70.5 |
| | | | | | | | | | | | 14.3 |

25 C.

| | | Recon | | | | | | | SEC Total | % Acidic |
|---|---|---|---|---|---|---|---|---|---|---|
| Time | Appearance, | Time | Conc | Turbidity | % | % | % | % | Peak Area | % Main |
| Point | pH | (min.) | (mg/mL) | A360 nm | Monomer | Dimer | Aggregate | LMW | Count | % Basic |
| 4 wk | II, Colorless | 2.2 | 38.1 | 0.040 | 99.22 | 0.72 | — | 0.06 | 34667 | 14.8 |
| | | | | | | | | | | 71.1 |
| | | | | | | | | | | 14.1 |
| 6 wk | II, Colorless | 2.8 | 38.6 | 0.044 | 99.19 | 0.75 | — | 0.06 | 35075 | 14.9 |
| | | | | | | | | | | 70.6 |
| | | | | | | | | | | 14.5 |
| 8 wk | II, BY7 | 3.0 | 38.3 | 0.053 | 99.12 | 0.80 | — | 0.08 | 34925 | 15.4 |
| | 6.35 | | | | | | | | | 70.2 |
| | | | | | | | | | | 14.4 |
| 12 wk | II, Colorless | 2.6 | 38.8 | 0.052 | 99.10 | 0.83 | — | 0.07 | 35107 | 15.5 |
| | | | | | | | | | | 70.0 |
| | | | | | | | | | | 14.5 |

40 C.

| | | Recon | | | | | | | SEC Total | % Acidic |
|---|---|---|---|---|---|---|---|---|---|---|
| Time | | Time | Conc | Turbidity | % | % | % | % | Peak Area | % Main |
| Point | Appearance | (min.) | (mg/mL) | A360 nm | Monomer | Dimer | Aggregate | LMW | Count | % Basic |
| 2 wk | II, | 2.8 | 37.4 | 0.046 | 99.22 | 0.78 | — | — | 34308 | 15.5 |
| | Colorless | | | | | | | | | 66.9 |
| | | | | | | | | | | 17.6 |
| 4 wk | II, | 1.9 | 37.7 | 0.052 | 98.82 | 1.11 | — | 0.07 | 35035 | 15.3 |
| | Colorless | | | | | | | | | 66.2 |
| | | | | | | | | | | 18.5 |
| 6 wk | II, | 5.9 | 38.5 | 0.043 | 98.68 | 1.25 | — | 0.07 | 34997 | 15.3 |
| | Colorless | | | | | | | | | 68.9 |
| | | | | | | | | | | 15.8 |

TABLE 44
100 mg/mL Antegren, 69.3 mg ml. Sucrose, 15 mM Histidine, 0.1% PS-80
1 vial tested per time point.
Prepared:
Lyophilization Complete: Testing Start Date:
At T = 0 reconstituted with 1.6 mL, all other time points
reconstituted with 1.3 mL
2-8 C.

| | | | | | | | | | | | % |
|---|---|---|---|---|---|---|---|---|---|---|---|
| | | Recon | | | | | | | | SEC Total | Acidic |
| Time | Appearance, | Time | KF % | Conc. | Turbidity | % | % | % | % | Peak Area | % Main |
| Point | pH | (min.) | Moisture | (mg/mL) | A360 nm | Monomer | Dimer | Aggregate | LMW | Count | % Basic |
| 0 | IV, | 2.5 | N/A | 85.2 | 0.100 | 99.49 | 0.51 | — | — | 36262 | 15.9 |
| | Colorless | | | | | | | | | | 67.0 |
| | | | | | | | | | | | 17.1 |
| 4 wk | III. | 3.6 | N/A | 107.5 | 0.112 | 99.27 | 0.55 | 0.11 | 0.07 | 36429 | 14.2 |
| | Colorless | | | | | | | | | | 71.2 |
| | | | | | | | | | | | 14.6 |
| 8 wk | IV, BY7 | 3.7 | N/A | 104.0 | 0.107 | 99.24 | 0.67 | — | 0.08 | 35154 | 15.3 |
| | 6.36 | | | | | | | | | | 70.6 |
| | | | | | | | | | | | 14.1 |
| 12 wk | III, BY7 | 4.1 | N/A | 100.4 | 0.105 | 99.22 | 0.72 | — | 0.06 | 35247 | 15.5 |
| | | | | | | | | | | | 70.4 |
| | | | | | | | | | | | 14.1 |

25 C.

| | | Recon | | | | | | | SEC Total | % Acidic |
|---|---|---|---|---|---|---|---|---|---|---|
| Time | Appearance, | Time | Conc. | Turbidity | % | % | % | % | Peak Area | % Main |
| Point | pH | (min.) | (mg/mL) | A360 nm | Monomer | Dimer | Aggregate | LMW | Count | % Basic |
| 4 wk | III, | 2.1 | 105.3 | 0.106 | 98.91 | 0.91 | 0.11 | 0.07 | 35903 | 14.9 |
| | Colorless | | | | | | | | | 70.7 |
| | | | | | | | | | | 14.4 |
| 6 wk | III, BY7 | 4.4 | 105.9 | 0.104 | 98.80 | 1.12 | — | 0.07 | 35374 | 14.9 |
| | | | | | | | | | | 69.8 |
| | | | | | | | | | | 15.4 |
| 8 wk | III, BY6 | 5.6 | 103.3 | 0.108 | 98.67 | 1.24 | — | 0.10 | 35697 | 15.3 |
| | 6.33 | | | | | | | | | 69.7 |
| | | | | | | | | | | 15.0 |
| 12 wk | III, BY7 | 5.5 | 95.2 | 0.123 | 98.53 | 1.39 | — | 0.08 | 34222 | 15.4 |
| | | | | | | | | | | 69.1 |
| | | | | | | | | | | 15.5 |

40 C.

| | | Recon | | | | | | | SEC Total | % Acidic |
|---|---|---|---|---|---|---|---|---|---|---|
| Time | | Time | Conc. | Turbidity | % | % | % | % | Peak Area | % Main |
| Point | Appearance | (min.) | (mg/mL) | A360 nm, | Monomer | Dimer | Aggregate | LMW | Count | % Basic |
| 2 wk | III, | 3.0 | 105.5 | 0.112 | 98.55 | 1.45 | — | — | 35690 | 15.4 |
| | Colorless | | | | | | | | | 66.8 |
| | | | | | | | | | | 17.8 |
| 4 wk | III, BY7 | 2.7 | 105.3 | 0.123 | 97.79 | 2.13 | — | 0.08 | 35694 | 15.4 |
| | | | | | | | | | | 64.5 |
| | | | | | | | | | | 20.1 |
| 6 wk | IV, BY7 | 3.4 | 105.0 | 0.115 | 97.29 | 2.61 | — | 0.09 | 35504 | 15.0 |
| | | | | | | | | | | 66.6 |
| | | | | | | | | | | 18.3 |

TABLE 45
40 mg/mL Antegren, 27.7 mg/mL Sucrose, 6 mM Histidine,
0.04% PS-80
1 vial tested per time point
Prepared:
Lyophilization Complete:
Testing Start Date:
At T = 0 reconstituted with 4.0 mL, all other time points
reconstituted with 3.9 mL.
2-8 C.

| | | Recon | | | | | | | | SEC Total | % Acidic |
|---|---|---|---|---|---|---|---|---|---|---|---|
| Time | Appearance. | Time | KF % | Conc. | Turbidity | % | % | % | % | Peak Area. | % Main |
| Point | pH | (min.) | Moisture | (mg/mL) | A360 nm | Monomer | Dimer | Aggregate | LMW | Count | % Basic |
| 0 | III, | 1.8 | 2.6 | 37.6 | 0.047 | 99.32 | 0.58 | — | 0.09 | 35322 | 16.0 |
| | Colorless | | | | | | | | | | 67.5 |
| | | | | | | | | | | | 16.5 |
| 4 wk | II, Colorless | 3.5 | N/A | 39.1 | 0.042 | 99.28 | 0.54 | 0.11 | 0.06 | 35887 | 14.1 |
| | | | | | | | | | | | 71.3 |
| | | | | | | | | | | | 14.6 |
| 8 wk | II, BY7 | 2.6 | N/A | 39.6 | 0.064 | 99.28 | 0.54 | 0.11 | 0.06 | 36694 | 15.0 |
| | 6.34 | | | | | | | | | | 70.1 |
| | | | | | | | | | | | 14.9 |
| 12 wk | II, Colorless | 2.0 | N/A | 40.2 | 0.045 | 99.24 | 0.69 | — | 0.07 | 35619 | 15.3 |
| | | | | | | | | | | | 70.5 |
| | | | | | | | | | | | 14.2 |

25 C.

| | | Recon | | | | | | | SEC Total | % Acidic |
|---|---|---|---|---|---|---|---|---|---|---|
| Time | Appearance, | Time | Conc. | Turbidity | % | % | % | % | Peak Area | % Main |
| Point | PH | (min.) | (mg/mL) | A360 nm | Monomer | Dimer | Aggregate | LMW | Count | % Basic |
| 4 wk | II, Colorless | 2.3 | 38.9 | 0.040 | 98.90 | 0.92 | — | 0.07 | 35695 | 14.8 |
| | | | | | | | | | | 70.8 |
| | | | | | | | | | | 14.4 |
| 6 wk | II, Colorless | 3.6 | 39.5 | 0.046 | 98.79 | 1.14 | — | 0.07 | 35977 | 14.8 |
| | | | | | | | | | | 69.8 |
| | | | | | | | | | | 15.3 |
| 8 wk | II, BY7 | 2.8 | 40.1 | 0.054 | 98.79 | 1.14 | — | 0.07 | 36710 | 15.6 |
| | 6.35 | | | | | | | | | 69.4 |
| | | | | | | | | | | 15.0 |
| 12 wk | II, Colorless | 2.7 | 37.9 | 0.046 | 98.54 | 1.39 | — | 0.07 | 35784 | 15.5 |
| | | | | | | | | | | 69.1 |
| | | | | | | | | | | 15.4 |

40 C.

| | | Recon | | | | | | | SEC Total | % Acidic |
|---|---|---|---|---|---|---|---|---|---|---|
| Time | Appearance, | Time | Conc. | Turbidity | % | % | % | % | Peak Area | % Main |
| Point | PH | (min.) | (mg/mL) | A360 nm | Monomer | Dimer | Aggregate | LMW | Count | % Basic |
| 2 wk | III, | 2.1 | 39.0 | 0.046 | 98.16 | 1.45 | — | 0.11 | 35830 | 15.5 |
| | Colorless | | | | | | | | | 66.3 |
| | | | | | | | | | | 18.2 |
| 4 wk | II, Colorless | 2.6 | 38.3 | 0.048 | 97.77 | 2.14 | — | 0.08 | 35603 | 15.3 |
| | | | | | | | | | | 64.2 |
| | | | | | | | | | | 20.6 |
| 6 wk | II, Colorless | 2.6 | 38.8 | 0.046 | 97.35 | 2.36 | 0.20 | 0.09 | 35888 | 15.0 |
| | | | | | | | | | | 66.7 |
| | | | | | | | | | | 18.3 |

TABLE 46
100 mg/nth Antegren, 23.0 mg mL Sucrose, 15 mM Histidine, 0.1% PS-80
1 vial tested per time point.
Prepared:
Lyophilization Complete:
Testing Start Date:
At T-0 reconstituted with 1.6 mL, all other time points
reconstituted with 1.3 mL.
2-8 C.

| | | Recon | | | | | | | | SEC Total | % Acidic |
|---|---|---|---|---|---|---|---|---|---|---|---|
| Time | Appearance. | Time | KF % | Conc. | Turbidity | % | % | % | % | Peak Area. | % Main |
| Point | pH | (min.) | Moisture | (mg/mL) | A360 nm | Monomer | Dimer | Aggregate | LMW | Count | % Basic |
| 0 | IV. | 4.9 | 5.7 | 85.8 | 0.109 | 99.22 | 0.76 | 0.03 | — | 36747 | 15.5 |
| | Colorless | | | | | | | | | | 67.2 |
| | | | | | | | | | | | 17.2 |
| 4 wk | III, | 2.6 | NIA | 107.9 | 0.120 | 98.94 | 0.99 | — | 0.07 | 36245 | 14.4 |
| | Colorless | | | | | | | | | | 70.8 |
| | | | | | | | | | | | 14.8 |
| 8 wk | IV, BY7 | 4.9 | N/A | 102.9 | 0.134 | 98.83 | 1.10 | — | 0.08 | 35153 | 15.1 |
| | 6.42 | | | | | | | | | | 69.4 |
| | | | | | | | | | | | 15.4 |
| 12 wk | IV, BY7 | 6.7 | N/A | 103.3 | 0.201 | 98.78 | 1.17 | — | 0.05 | 35780 | 15.5 |
| | | | | | | | | | | | 69.6 |
| | | | | | | | | | | | 14.9 |

25 C.

| | | Recon | | | | | | | SEC Total | % Acidic |
|---|---|---|---|---|---|---|---|---|---|---|
| Time | Appearance, | Time | Conc. | Turbidity | % | | % | % | Peak Area | % Main |
| Point | PH | (min.) | (mg/mL) | A360 nm | Monomer | % Dimer | Aggregate | LMW | Count | % Basic |
| 4 wk | IV, | 2.3 | 106.8 | 0.129 | 97.57 | 2.17 | 0.17 | 0.09 | 35755 | 15.1 |
| | Colorless | | | | | | | | | 68.9 |
| | | | | | | | | | | 16.0 |
| 6 wk | IV, BY7 | 5.1 | 107.5 | 0.148 | 97.20 | 252 | 0.19 | 0.09 | 35963 | 14.8 |
| | | | | | | | | | | 67.2 |
| | | | | | | | | | | 18.0 |
| 8 wk | IV, BY6 | 3.8 | 106.5 | 0.124 | 96.71 | −2.95 | 0.25 | 0.09 | 35546 | 15.7 |
| | 6.33 | | | | | | | | | 65.7 |
| | | | | | | | | | | 18.6 |
| 12 wk | III, BY7 | 5.9 | 99.7 | 0.122 | 96.22 | 3.39 | 0.28 | 0.10 | 35334 | 15.4 |
| | | | | | | | | | | 65.9 |
| | | | | | | | | | | 18.7 |

40 C.

| | | Recon | | | | | | | SEC Total | % Acidic |
|---|---|---|---|---|---|---|---|---|---|---|
| Time | | Time | Conc. | Turbidity | % | | % | % | Peak Area | % Main |
| Point | Appearance | (min.) | (mg/mL) | A360 nm | Monomer | % Dimer | Aggregate | LMW | Count | % Basic |
| 2 wk | IV, | 2.5 | 105.9 | 0.133 | 95.76 | 3.93 | 0.30 | — | 35826 | 15.5 |
| | Colorless | | | | | | | | | 59.6 |
| | | | | | | | | | | 24.9 |
| 4 wk | IV, BY7 | 3.8 | 106.1 | 0.132 | 93.46 | 5.80 | 0.63 | 0.10 | 36136 | 145 |
| | | | | | | | | | | 55.0 |
| | | | | | | | | | | 30.5 |
| 6 wk | IV, | 5.6 | 108.3 | 0.139 | 91.91 | 7.09 | 0.89 | 0.11 | 36007 | 14.7 |
| | Colorless | | | | | | | | | 57.8 |
| | | | | | | | | | | 27.4 |

TABLE 47
40 mg/mL Antegren, 9.2 mg/mL Sucrose, 6 mM Histidine, 0.04% PS-80
1 vial tested per time point.
Prepared;
Lyophilization Complete:
Testing Start Date:.
At T = 0 reconstituted with 4.0 mL, all other time points
reconstituted with 3.9 mL.
2-8 C.

| | | Recon | | | | | | | | SEC Total | % Acidic |
|---|---|---|---|---|---|---|---|---|---|---|---|
| Time | Appearance, | Time | KF % | Conc. | Turbidity | % | % | % | % | Peak Area | % Main |
| Point | pH | (min.) | Moisture | (mg/mL) | A360 nm | Monomer | Dimer | Aggregate | LMW | Count | % Basic |
| 0 | III, | 2.2 | 5.0 | 36.6 | 0.082 | 99.24 | 0.76 | — | — | 34010 | 15.8 |
| | Colorless | | | | | | | | | | 67.4 |
| | | | | | | | | | | | 16.9 |
| 4 wk | II, Colorless | 2.8 | N/A | 38.8 | 0.053 | 98.92 | 1.01 | — | 0.07 | 35211 | 14.4 |
| | | | | | | | | | | | 71.1 |
| | | | | | | | | | | | 14.5 |
| 8 wk | II, Colorless | 2.6 | N/A | 39.3 | 0.052 | 98.84 | 1.09 | — | 0.07 | 36176 | 15.1 |
| | 6.38 | | | | | | | | | | 69.9 |
| | | | | | | | | | | | 15.0 |
| 12 wk | II, Colorless | 1.9 | N/A | 40.8 | 0.069 | 98.75 | 1.18 | — | 0.07 | 35825 | 15.4 |
| | | | | | | | | | | | 69.7 |
| | | | | | | | | | | | 14.9 |

25 C.

| | | Recon | | | | | | | SEC Total | % Acidic |
|---|---|---|---|---|---|---|---|---|---|---|
| Time | Appearance, | Time | Conc. | Turbidity | % | % | % | % | Peak Area | % Main |
| Point | pH | (min.) | (mg/mL) | A360 nm | Monomer | Dimer | Aggregate | LMW | Count | % Basic |
| 4 wk | II, Colorless | 2.1 | 38.5 | 0.046 | 97.58 | 2.14 | 0.19 | 0.08 | 35236 | 15.0 |
| | | | | | | | | | | 68.9 |
| | | | | | | | | | | 16.1 |
| 6 wk | II, Colorless | 3.4 | 38.6 | 0.053 | 97.22 | 2.48 | 0.21 | 0.09 | 34993 | 14.8 |
| | | | | | | | | | | 67.4 |
| | | | | | | | | | | 17.8 |
| 8 wk | III, BY7 | 3.0 | 39.0 | 0.063 | 96.73 | 2.91 | 0.25 | 0.11 | 35998 | 15.1 |
| | 6.30 | | | | | | | | | 66.9 |
| | | | | | | | | | | 18.0 |
| 12 wk | II, Colorless | 2.1 | 39.1 | 0.049 | 96.25 | 3.37 | 0.29 | 0.10 | 35465 | 15.5 |
| | | | | | | | | | | 65.9 |
| | | | | | | | | | | 18.6 |

40 C.

| | | Recon | | | | | | | SEC Total | % Acidic |
|---|---|---|---|---|---|---|---|---|---|---|
| Time | | Time | Conc. | Turbidity | % | % | % | % | Peak Area | % Main |
| Point | Appearance | (min.) | (mg/mL) | A360 nm | Monomer | Dimer | Aggregate | LMW | Count | % Basic |
| 2 wk | II, | 2.1 | 38.5 | 0.052 | 95.52 | 3.99 | 0.32 | 0.17 | 35285 | 15.3 |
| | Colorless | | | | | | | | | 59.3 |
| | | | | | | | | | | 25.3 |
| 4 wk | II, | 3.3 | 38.4 | 0.047 | 93.58 | 5.71 | 0.61 | 0.10 | 35716 | 14.7 |
| | Colorless | | | | | | | | | 58.6 |
| | | | | | | | | | | 26.7 |
| 6 wk | III. | 43 | 38.7 | 0.055 | 92.17 | 6.90 | 0.84 | 0.10 | 35585 | 14.8 |
| | Colorless | | | | | | | | | 58.9 |
| | | | | | | | | | | 26.3 |

TABLE 48
160 mg/mL Antegren, 36.8 mg/mL Sucrose, 24 mM Histidine, 0.16% PS-80
1 vial tested per time point.
Prepared.
Lyophiliiation Complete
Testing Start Date:
At T = 0 reconstituted with 1.0 mL, all other time points
reconstituted with 0.8 mL.
2-8 C.

| | | Recon | | | | | | | | SEC Total | % Acidic |
|---|---|---|---|---|---|---|---|---|---|---|---|
| Time | Appearance, | Time | KF % | Conc. | Turbidity | % | % | % | % | Peak Area | % Main |
| Point | PH | (min.) | Moisture | (mg/mL) | A360 nm | Monomer | Dimer | Aggregate | LMW | Count | % Basic |
| 0 | IV, | 6.0 | 5.5 | 128.8 | 0.162 | 99.23 | 0.77 | — | — | 33210 | 16.0 |
| | Colorless | | | | | | | | | | 66.5 |
| | | | | | | | | | | | 17.5 |
| 4 wk | IV, | 7.8 | N/A | 153.4 | 0.140 | 98.90 | 1.02 | — | 0.08 | 40638 | 14.5 |
| | Colorless | | | | | | | | | | 70.6 |
| | | | | | | | | | | | 14.9 |
| 8 wk | IV, BY7 | 8.0 | N/A | 153.4 | 0.150 | 98.79 | 1.14 | — | 0.08 | 40900 | 15.6 |
| | 6.43 | | | | | | | | | | 69.4 |
| | | | | | | | | | | | 15.0 |
| 12 wk | IV, BY7 | 6.4 | N/A | 143.9 | 0.163 | 98.72 | 1.21 | — | 0.07 | 40819 | 15.4 |
| | | | | | | | | | | | 69.7 |
| | | | | | | | | | | | 14.9 |

25 C.

| | | Recon | | | | | | | SEC Total | % Acidic |
|---|---|---|---|---|---|---|---|---|---|---|
| Time | Appearance, | Time | Conc. | Turbidity | % | % | % | | Peak Area | % Main |
| Point | PH | (min.) | (mg/mL) | A360 nm | Monomer | Dimer | Aggregate | % LMW | Count | % Basic |
| 4 wk | IV. | 5.6 | 153.1 | 0.144 | 97.53 | 2.20 | 0.18 | 0.09 | 40260 | 15.2 |
| | Colorless | | | | | | | | | 69.0 |
| | | | | | | | | | | 15.8 |
| 6 wk | IV, BY7 | 7.6 | 156.6 | 0.179 | 97.15 | 2.56 | 0.20 | 0.09 | 40334 | 14.9 |
| | | | | | | | | | | 67.3 |
| | | | | | | | | | | 17.8 |
| 8 wk | IV, BY6 | 5.7 | 153.3 | 0.155 | 96.67 | 2.96 | 0.26 | 0.11 | 40584 | 15.3 |
| | 6.35 | | | | | | | | | 67.6 |
| | | | | | | | | | | 17.1 |
| 12 wk | IV, BY7 | 9.8 | 131.7 | 0.136 | 96.16 | 3.44 | 0.29 | 0.10 | 40606 | 15.4 |
| | | | | | | | | | | 66.0 |
| | | | | | | | | | | 18.6 |

40 C.

| | | Recon | | | | | | | SEC Total | % Acidic |
|---|---|---|---|---|---|---|---|---|---|---|
| Time | | Time | Conc. | Turbidity | % | % | % | | Peak Area | % Main |
| Point | Appearance | (min.) | (mg/mL) | A360 nm | Monomer | Dimer | Aggregate | % LMW | Count | % Basic |
| 2 wk | IV. | 8.7 | 156.8 | 0.163 | 95.67 | 4.02 | 0.30 | — | 40618 | 15.0 |
| | Colorless | | | | | | | | | 59.8 |
| | | | | | | | | | | 25.2 |
| 4 wk | IV, BY7 | 7.9 | 151.2 | 0.157 | 93.42 | 5.82 | 0.65 | 0.11 | 40641 | 14.5 |
| | | | | | | | | | | 55.1 |
| | | | | | | | | | | 30.4 |
| 6 wk | IV, BY7 | 9.1 | 156.0 | 0.152 | 91.75 | 7.21 | 0.93 | 0.11 | 40735 | 14.9 |
| | | | | | | | | | | 57.7 |
| | | | | | | | | | | 27.4 |

100 mg mL Antegren. 69.3 mg/m.. Sucrose, 15 mM Histidine, 0.1% PS-80

TABLE 49
100 mg/mL Antegren, 69.3 mg/mL Sucrose, 15 mM Histidine, 0.1% PS-80
1 vial tested per time point.
Prepared.
Lyophiliiation Complete
Testing Start Date:
At T = 0 reconstituted with 1.6 mL, all other time points
reconstituted with 1.3 mL.
2-8 C.

| | | Recon | | | | | | | SEC Total | |
|---|---|---|---|---|---|---|---|---|---|---|
| Time | Appearances, | Time | Conc. | Turbidity | % | | % | | Peak Area | % Acidic % |
| Point | pH | (min.) | (mg/mL) | A360 nm | Monomer | % Dimer | Aggregate | % LMW | Count | Main % Basic |
| 4 wk | III, Colorless | 3.0 | 106.1 | 0.098 | 98.91 | 1.02 | — | 0.07 | 35758 | 15.2 |
| | | | | | | | | | | 70.7 |
| | | | | | | | | | | 14.1 |
| 6 wk | III, Colorless | 5.2 | 103.3 | 0.100 | 98.80 | 1.14 | — | 0.07 | 35025 | 15.2 |
| | | | | | | | | | | 69.9 |
| | | | | | | | | | | 14.9 |
| 8 wk | IV, BY6 | 4.0 | 103.5 | 0.108 | 98.67 | 1.24 | — | 0.09 | 34972 | 14.9 |
| | 6.35 | | | | | | | | | 69.9 |
| | | | | | | | | | | 15.2 |
| 12 wk | III, BY7 | 4.5 | 100.9 | 0.101 | 98.55 | 1.37 | — | 0.08 | 35358 | 15.6 |
| | | | | | | | | | | 69.1 |
| | | | | | | | | | | 15.2 |

40 C.

| | | Recon | | | | | | | SEC Total | |
|---|---|---|---|---|---|---|---|---|---|---|
| Time | Appearances, | Time | Conc. | Turbidity | % | | % | | Peak Area | % Acidic % |
| Point | pH | (min.) | (mg/mL) | A360 nm | Monomer | % Dimer | Aggregate | % LMW | Count | Main % Basic |
| 2 wk | III, Colorless | 2.7 | 105.0 | 0.102 | 98.54 | 1.46 | — | — | 35940 | 15.6 |
| | | | | | | | | | | 66.7 |
| | | | | | | | | | | 17.7 |
| 4 wk | III, BY7 | 2.6 | 85.4 | 0.097 | 97.82 | 2.10 | — | 0.08 | 29414 | 15.3 |
| | | | | | | | | | | 64.0 |
| | | | | | | | | | | 20.8 |
| 6 wk | III, Colorless | 2.9 | 106.3 | 0.100 | 97.33 | 2.36 | 0.21 | 0.09 | 35794 | 15.4 |
| | | | | | | | | | | 66.4 |
| | | | | | | | | | | 18.2 |

TABLE 50
100 mg/mL Antegren, 115.5 mg/mL Sucrose, 15 mM Histidine, 0.1% PS-80
1 vial tested per time point.
Prepared.
Lyophiliiation Complete
Testing Start Date:
At T = 0 reconstituted with 1.6 mL, all other time points
reconstituted with 1.3 mL.
2-8 C.

| 0 | IV, Colorless | 2.9 | N/A | 72.6 | 0.089 | 99.48 | 0.52 | — | — | 36877 | 15.5 |
|---|---|---|---|---|---|---|---|---|---|---|---|
| | | | | | | | | | | | 67.1 |
| | | | | | | | | | | | 17.4 |
| 4 wk | III, Colorless | 3.4 | N/A | 104.7 | 0.100 | 99.28 | 0.55 | 0.11 | 0.06 | 35319 | 14.8 |
| | | | | | | | | | | | 71.3 |
| | | | | | | | | | | | 13.9 |
| 8 wk | III, BY7 | 4.7 | N/A | 102.3 | 0.106 | 99.23 | 0.68 | — | 0.08 | 34784 | 15.6 |
| | 6.42 | | | | | | | | | | 70.1 |
| | | | | | | | | | | | 14.2 |
| 12 wk | III, BY7 | 5.8 | N/A | 100.5 | 0.104 | 99.21 | 0.72 | — | 0.07 | 35667 | 15.6 |
| | | | | | | | | | | | 70.6 |
| | | | | | | | | | | | 13.8 |
| | | Recon | | | | | | | | SEC Total | |
| Time | Appearances, | Time | KF % | Conc. | Turbidity | % | | % | % | Peak Area | % Acidic % |
| Point | pH | (min.) | Moisture | (mg/mL) | A360 nm | Monomer | % Dimer | Aggregate | LMW | Count | Main % Basic |
| 0 | IV, Colorless | 6.7 | 2.0 | 82.0 | 0.090 | 99.54 | 0.46 | — | — | 35236 | 15.4 |
| | | | | | | | | | | | 67.6 |
| | | | | | | | | | | | 17.0 |
| 4 wk | III, Colorless | 3.9 | N/A | 100.6 | 0.085 | 99.37 | 0.46 | 0.11 | 0.06 | 34035 | 14.8 |
| | | | | | | | | | | | 71.5 |
| | | | | | | | | | | | 13.7 |
| 8 wk | III, BY7 | 6.7 | N/A | 97.3 | 0.096 | 99.33 | 0.58 | — | 0.09 | 33404 | 15.5 |
| | 6.44 | | | | | | | | | | 70.9 |
| | | | | | | | | | | | 13.6 |
| 12 wk | III, BY7 | 8.7 | N/A | 100.5 | 0.092 | 99.34 | 0.58 | — | 0.08 | 34370 | 15.7 |
| | | | | | | | | | | | 70.7 |
| | | | | | | | | | | | 13.6 |

| | | | | | | | | | | |
|---|---|---|---|---|---|---|---|---|---|---|
| | | Recon | | | | | | | SEC Total | |
| Time | Appearances, | Time | Conc. | Turbidity | % | | % | | Peak Area | % Acidic % |
| Point | pH | (min.) | (mg/mL) | A360 nm | Monomer | % Dimer | Aggregate | % LMW | Count | Main % Basic |

25 C.

| | | | | | | | | | | |
|---|---|---|---|---|---|---|---|---|---|---|
| 4 wk | III, Colorless | 3.6 | 100.9 | 0.096 | 99.22 | 0.71 | — | 0.06 | 34149 | 15.3 |
| | | | | | | | | | | 71.2 |
| | | | | | | | | | | 13.5 |
| 6 wk | III, Colorless | 3.4 | 100.1 | 0.088 | 99.18 | 0.75 | — | 0.06 | 34039 | 15.3 |
| | | | | | | | | | | 70.6 |
| | | | | | | | | | | 14.1 |
| 8 wk | III, BY6 | 4.9 | 99.0 | 0.097 | 99.14 | 0.76 | — | 0.07 | 33713 | 15.8 |
| | 6.37 | | | | | | | | | 70.1 |
| | | | | | | | | | | 14.2 |
| 12 wk | III, BY7 | 4.4 | 94.5 | 0.100 | 99.09 | 0.83 | — | 0.07 | 33713 | 15.8 |
| | | | | | | | | | | 70.1 |
| | | | | | | | | | | 14.2 |

40 C.

| | | | | | | | | | | |
|---|---|---|---|---|---|---|---|---|---|---|
| 2 wk | III, Colorless | 3.8 | 98.1 | 0.089 | 99.22 | 0.78 | — | — | 33705 | 15.8 |
| | | | | | | | | | | 67.1 |
| | | | | | | | | | | 17.0 |
| 4 wk | III, BY7 | 3.1 | 97.5 | 0.092 | 98.81 | 1.11 | — | 0.08 | 34307 | 15.5 |
| | | | | | | | | | | 66.3 |
| | | | | | | | | | | 18.2 |
| 6 wk | III, Colorless | 7.4 | 102.4 | 0.091 | 98.68 | 1.25 | — | 0.07 | 34449 | 15.6 |
| | | | | | | | | | | 69.1 |
| | | | | | | | | | | 15.3 |

TABLE 51

| | | |
|---|---|---|
| A | B | C |

% HMW

| | | | |
|---|---|---|---|
| 0 | 0.49 | 0.39 | 0.43 |
| 8 | 0.5 | 0.49 | 0.51 |
| 12 | 0.5 | 0.49 | 0.50 |

% LMW

| | | | |
|---|---|---|---|
| 0 | 0.09 | 0 | 0.00 |
| 8 | 0.08 | 0.08 | 0.07 |
| 12 | 0.07 | 0.07 | 0.06 |

% Monomer

| | | | |
|---|---|---|---|
| 0 | 99.42 | 99.61 | 99.57 |
| 8 | 99.43 | 99.43 | 99.42 |
| 12 | 99.43 | 99.44 | 99.43 |

TABLE 52
Lyo
40 C.

| | | | | | | | | | | |
|---|---|---|---|---|---|---|---|---|---|---|
| A1 | A2 | B3 | B4 | A5 | A6 | C7 | C8 | C9 | A10 | B11 |

% HMW

| | | | | | | | | | | | |
|---|---|---|---|---|---|---|---|---|---|---|---|
| 0 | 0.50 | 0.51 | 0.45 | 0.44 | 0.51 | 0.58 | 0.79 | 0.76 | 0.77 | 0.52 | 0.46 |
| 2 | 1.48 | 1.47 | 0.77 | 0.78 | 1.45 | 1.45 | 4.23 | 4.31 | 4.32 | 1.46 | 0.78 |
| 4 | 2.10 | 2.16 | 1.08 | 1.11 | 2.13 | 2.14 | 6.43 | 6.32 | 6.47 | 2.10 | 1.11 |
| 6 | 2.62 | 2.59 | 1.27 | 1.25 | 2.61 | 2.56 | 7.98 | 7.74 | 8.14 | 2.57 | 1.25 |

% LMW

| | | | | | | | | | | | |
|---|---|---|---|---|---|---|---|---|---|---|---|
| 0 | 0.00 | 0.00 | 0.00 | 0.00 | 0.00 | 0.09 | 0.00 | 0.00 | 0.00 | 0.00 | 0.00 |
| 2 | 0.00 | 0.00 | 0.00 | 0.00 | 0.00 | 0.11 | 0.00 | 0.17 | 0.00 | 0.00 | 0.00 |
| 4 | 0.09 | 0.08 | 0.08 | 0.07 | 0.08 | 0.08 | 0.10 | 0.10 | 0.11 | 0.08 | 0.08 |
| 6 | 0.10 | 0.09 | 0.08 | 0.07 | 0.09 | 0.09 | 0.11 | 0.10 | 0.11 | 0.09 | 0.07 |

% Monomer

| | | | | | | | | | | | |
|---|---|---|---|---|---|---|---|---|---|---|---|
| 0 | 99.50 | 99.49 | 99.55 | 99.56 | 99.49 | 99.32 | 99.22 | 99.24 | 99.23 | 99.48 | 99.54 |
| 2 | 98.52 | 98.53 | 99.23 | 99.22 | 98.55 | 98.16 | 95.76 | 95.52 | 95.67 | 98.54 | 99.22 |
| 4 | 97.81 | 97.76 | 98.84 | 98.82 | 97.79 | 97.77 | 93.46 | 93.58 | 93.42 | 97.82 | 98.81 |
| 6 | 97.28 | 97.32 | 98.65 | 98.68 | 97.29 | 97.35 | 91.91 | 92.17 | 91.75 | 97.33 | 98.68 |

TABLE 53
Lyo
25 C.

| | | | | | | | | | | |
|---|---|---|---|---|---|---|---|---|---|---|
| A1 | A2 | B3 | B4 | A5 | A6 | C7 | C8 | C9 | A10 | B11 |

% HMW

| | | | | | | | | | | | |
|---|---|---|---|---|---|---|---|---|---|---|---|
| 0 | 0.50 | 0.51 | 0.45 | 0.44 | 0.51 | 0.58 | 0.79 | 0.76 | 0.77 | 0.52 | 0.46 |
| 4 | 1.03 | 1.02 | 0.70 | 0.72 | 1.02 | 0.92 | 2.34 | 2.33 | 2.38 | 1.02 | 0.71 |
| 6 | 1.13 | 1.12 | 0.75 | 0.75 | 1.12 | 1.14 | 2.71 | 2.69 | 2.76 | 1.14 | 0.75 |
| 8 | 1.25 | 1.23 | 0.80 | 0.80 | 1.24 | 1.27 | 3.20 | 3.16 | 3.22 | 1.24 | 0.76 |
| 12 | 1.40 | 1.38 | 0.82 | 0.83 | 1.39 | 1.39 | 3.67 | 3.66 | 3.73 | 1.37 | 0.83 |

% LMW

| | | | | | | | | | | | |
|---|---|---|---|---|---|---|---|---|---|---|---|
| 0 | 0.00 | 0.00 | 0.00 | 0.00 | 0.00 | 0.09 | 0.00 | 0.00 | 0.00 | 0.00 | 0.00 |
| 4 | 0.07 | 0.08 | 0.07 | 0.06 | 0.07 | 0.07 | 0.09 | 0.08 | 0.09 | 0.07 | 0.06 |
| 6 | 0.08 | 0.07 | 0.07 | 0.06 | 0.07 | 0.07 | 0.09 | 0.09 | 0.09 | 0.07 | 0.06 |
| 8 | 0.11 | 0.09 | 0.09 | 0.08 | 0.10 | 0.08 | 0.09 | 0.11 | 0.11 | 0.09 | 0.10 |
| 12 | 0.08 | 0.08 | 0.08 | 0.07 | 0.08 | 0.07 | 0.10 | 0.10 | 0.10 | 0.08 | 0.07 |

% Monomer

| | | | | | | | | | | | |
|---|---|---|---|---|---|---|---|---|---|---|---|
| 0 | 99.50 | 99.49 | 99.55 | 99.56 | 99.49 | 99.32 | 99.22 | 99.24 | 99.23 | 99.48 | 99.54 |
| 4 | 98.89 | 98.90 | 99.22 | 99.22 | 98.91 | 98.90 | 97.57 | 97.58 | 97.53 | 98.91 | 99.22 |
| 6 | 98.79 | 98.81 | 99.18 | 99.19 | 98.80 | 98.79 | 97.20 | 97.22 | 97.15 | 98.80 | 99.18 |
| 8 | 98.64 | 98.67 | 99.11 | 99.12 | 98.67 | 98.65 | 96.71 | 96.73 | 96.67 | 98.67 | 99.14 |
| 12 | 98.52 | 98.55 | 99.10 | 99.10 | 98.53 | 98.54 | 96.22 | 96.25 | 96.16 | 98.55 | 99.09 |

TABLE 54
Lyo
25 C.

| | | | | | | | | | | |
|---|---|---|---|---|---|---|---|---|---|---|
| A | A2 | B3 | B4 | A5 | A6 | C7 | C8 | C9 | A10 | B11 |

% HMW

| | | | | | | | | | | | |
|---|---|---|---|---|---|---|---|---|---|---|---|
| 0 | 0.50 | 0.51 | 0.45 | 0.44 | 0.51 | 0.58 | 0.79 | 0.76 | 0.77 | 0.52 | 0.46 |
| 4 | 0.65 | 0.65 | 0.56 | 0.59 | 0.66 | 0.65 | 0.99 | 1.01 | 1.02 | 0.66 | 0.57 |
| 8 | 0.68 | 0.67 | 0.57 | 0.58 | 0.67 | 0.67 | 1.10 | 1.09 | 1.14 | 0.68 | 0.58 |
| 12 | 0.71 | 0.72 | 0.60 | 0.58 | 0.72 | 0.69 | 1.17 | 1.18 | 1.21 | 0.72 | 0.58 |

% LMW

| | | | | | | | | | | | |
|---|---|---|---|---|---|---|---|---|---|---|---|
| 0 | 0.00 | 0.00 | 0.00 | 0.00 | 0.00 | 0.09 | 0.00 | 0.00 | 0.00 | 0.00 | 0.00 |
| 4 | 0.07 | 0.06 | 0.07 | 0.06 | 0.07 | 0.06 | 0.07 | 0.07 | 0.08 | 0.06 | 0.06 |
| 8 | 0.10 | 0.08 | 0.10 | 0.07 | 0.08 | 0.07 | 0.08 | 0.07 | 0.08 | 0.08 | 0.09 |
| 12 | 0.08 | 0.07 | 0.09 | 0.07 | 0.06 | 0.07 | 0.05 | 0.07 | 0.07 | 0.07 | 0.08 |

% Monomer

| | | | | | | | | | | | |
|---|---|---|---|---|---|---|---|---|---|---|---|
| 0 | 99.50 | 99.49 | 99.55 | 99.56 | 99.49 | 99.32 | 99.22 | 99.24 | 99.23 | 99.48 | 99.54 |
| 4 | 99.27 | 99.28 | 99.37 | 99.35 | 99.27 | 99.28 | 98.94 | 98.92 | 98.90 | 99.28 | 99.37 |
| 8 | 99.22 | 99.25 | 99.34 | 99.34 | 99.24 | 99.25 | 98.83 | 98.84 | 98.79 | 99.23 | 99.33 |
| 12 | 99.21 | 99.21 | 99.32 | 99.35 | 99.22 | 99.24 | 98.78 | 98.75 | 98.72 | 99.21 | 99.34 |

TABLE 55
Pre-Lyo A

5 C.

Pre-Lyo B

5 C.

Pre-Lyo C.

5 C.

A1

| | |
|---|---|
| 5 C. | Stayed IV, changed to BY7 at 8 wk |
| 25 C. | Stayed IV, changed to BY7 at 6 wk |
| 40 C. | Stayed IV, changed to BY7 at 4 wk |

A2

| | |
|---|---|
| 5 C. | Stayed III, changed to BY7 at 8 wk |
| 25 C. | Stayed III, changed to BY7 at 6 wk |
| 40 C. | Changed to IV at 6 wk, changed to BY7 at 4 wk |

B3

| | |
|---|---|
| 5 C. | Stayed III, changed to BY7 at 8 wk |
| 25 C. | Changed to IV at 12 wk, changed to BY7 at 6 wk |
| 40 C. | Stayed III, changed to BY7 at 4 wk |

B4

| | |
|---|---|
| 5 C. | No major changes, all stayed at II, colorless |
| 25 C. | |
| 40 C. | |

A5

5 C.
25 C.
40 C.

A6

5 C.
25 C.
40 C.

C7

5 C.
25 C.
40 C.

C8

5 C.
25 C.
40 C.

C9

5 C.
25 C.
40 C.

A10

5 C.
25 C.
40 C.

B11

5 C.
25 C.
40 C.

TABLE 56
No major changes seen in turbidity at any temperature.

| | | | | | | | | | | |
|---|---|---|---|---|---|---|---|---|---|---|
| A1 | A2 | B3 | B4 | A5 | A6 | C7 | C8 | C9 | A10 | B11 |

40 C.

| | | | | | | | | | | | |
|---|---|---|---|---|---|---|---|---|---|---|---|
| 0 | 0.126 | 0.095 | 0.112 | 0.054 | 0.100 | 0.047 | 0.109 | 0.082 | 0.162 | 0.089 | 0.090 |
| 2 | 0.124 | 0.106 | 0.107 | 0.046 | 0.112 | 0.046 | 0.133 | 0.052 | 0.163 | 0.102 | 0.089 |
| 4 | 0.113 | 0.110 | 0.102 | 0.052 | 0.123 | 0.048 | 0.132 | 0.047 | 0.157 | 0.097 | 0.092 |
| 6 | 0.113 | 0.111 | 0.114 | 0.043 | 0.115 | 0.046 | 0.139 | 0.055 | 0.152 | 0.100 | 0.091 |

25 C.

| | | | | | | | | | | | |
|---|---|---|---|---|---|---|---|---|---|---|---|
| 0 | 0.126 | 0.095 | 0.112 | 0.054 | 0.100 | 0.047 | 0.109 | 0.082 | 0.162 | 0.089 | 0.090 |
| 4 | 0.111 | 0.101 | 0.093 | 0.040 | 0.106 | 0.040 | 0.129 | 0.046 | 0.144 | 0.098 | 0.096 |
| 6 | 0.132 | 0.106 | 0.109 | 0.044 | 0.104 | 0.046 | 0.148 | 0.053 | 0.179 | 0.100 | 0.088 |
| 8 | 0.116 | 0.101 | 0.099 | 0.053 | 0.108 | 0.054 | 0.124 | 0.063 | 0.155 | 0.108 | 0.097 |
| 12 | 0.119 | 0.105 | 0.118 | 0.052 | 0.123 | 0.046 | 0.122 | 0.049 | 0.136 | 0.101 | 0.100 |

5 C.

| | | | | | | | | | | | |
|---|---|---|---|---|---|---|---|---|---|---|---|
| 0 | 0.126 | 0.095 | 0.112 | 0.054 | 0.100 | 0.047 | 0.109 | 0.082 | 0.162 | 0.089 | 0.090 |
| 4 | 0.101 | 0.111 | 0.124 | 0.041 | 0.112 | 0.042 | 0.120 | 0.053 | 0.140 | 0.100 | 0.085 |
| 8 | 0.110 | 0.107 | 0.105 | 0.046 | 0.107 | 0.064 | 0.134 | 0.052 | 0.150 | 0.106 | 0.096 |
| 12 | 0.108 | 0.107 | 0.116 | 0.051 | 0.105 | 0.045 | 0.201 | 0.069 | 0.163 | 0.104 | 0.092 |

Pre-Lyo 5 C

| | A | B | C |
|---|---|---|---|
| 0 | 0.044 | 0.041 | 0.041 |
| 8 | 0.053 | 0.040 | 0.041 |
| 12 | 0.041 | 0.044 | 0.043 |

Also refer to Examples 25A, B, C, and D.

TABLE 57
Recon Stability

| 4 wks at 40 C. | | | 4 wks at 5 C | | |
|---|---|---|---|---|---|
| 4089-1L % monomer | | | 4089-1L % monomer | | |
| 5 C. | 0 | 96.5 | 5 C. | 0 | 97.78 |
| | 0 | 96.56 | | 0 | 97.77 |
| | 1 | 96.57 | | 1 | 97.8 |
| | 1 | 96.61 | | 1 | 97.79 |
| 25 C. | 0 | 96.55 | 25 C. | 0 | |
| | 0 | 96.52 | | 0 | |
| | 1 | 96.72 | | 1 | |
| | 1 | 96.7 | | 1 | |

4 wks at 40 C.
4089-1N % monomer

| | | |
|---|---|---|
| 5 C. | 0 | 95.87 |
| | 0 | 95.90 |
| | 1 | 95.92 |
| | 1 | 95.97 |
| 25 C. | 0 | 95.86 |
| | 0 | 95.87 |
| | 1 | 96.06 |
| | 1 | 96.07 |

### Example 5

The formulation described herein is a lyophilized cake containing natalizumab, sucrose, histidine and polysorbate 80. The pre-lyophilized bulk drug substance contains 40 mg/mL antibody, 41 mg/mL sucrose, 0.04% polysorbate 80 and 6 mM histidine HCI, pH 6.0. This was filled at 4 mL per vial lyophilized, reconstituted with 1.0 mL water to 120 mg/mL, 123 mg/mL sucrose, 0.12% polysorbate 80 and 18 mM histidine HCI, pH 6.0.

Pre-lyophilized natalizumab composition was provided by BiogenIdec. It was formulated into phosphate buffered saline and was processed into an ammonium sulfate solution. This material was then diafiltered into the formulation buffer (without polysorbate) and concentrated to 40 mg/mL. The appropriate amount of polysorbate 80 was then added, the material was sterile filtered into polypropylene bottles and stored at 2-8° C. for 4 weeks prior to filling and lyophilization. Filling and lyophilization were performed in a non-GMP suite using a written batch record. The suite was sanitized prior to filling and all filling operations were performed under a laminar flow hood. Lyophilization was performed using a Virtis Gensis 25 EL lyophilizer.

The stability study consists of three arms, one examined the stability of the pre-lyophilized natalizumab composition for up to one year at the recommended storage temperature (2-8° C.) and up to 6 months at an accelerated temperature of 25° C. The lyophilized composition vials stored for 12 months at the recommended storage temperature (2-8° C.), 6 months at 25° C. and 3 months at 40° C. Vials at various timepoints from the 2-8° C. arm were reconstituted and stored for 1 week at both 2-8° C. (recommended temperature) and 25° C. (accelerated).

Quality control stability tests were performed with regard to appearance, A280, pH, non-reduced and reduced SDS-PAGE, cation exchange chromatography, and size exclusion chromatography.

### Material, Methods and Testing Time Points

The following samples will be tested

TABLE 58
Sample Configuration

| Batch/Lot | Description |
|---|---|
| Pre-lyophilized Biogenldec | 2 mL fill in polypropylene vials |
| Lot #: LYO121504 | |
| Lyophilized vials Lot# | 5 mL vials containing lyophilized cake with |
| K65001 containing 160 mg | butyl rubber stopper and Flip-off cap. Vials |
| of active/vial | to be reconstituted with 1 mL of WFI or |
| | other purified water. |

TABLE 59
Study Methods

| | | Volume | |
|---|---|---|---|
| Method Number | Title | Needed | |
| EOP 000-01291 | Determination of Appearance of | 2 | vials* |
| | Lyophilized Vials | | |
| EOP 000-01291 | Moisture | 2 | vials |
| EOP 000-01292 | Determination of Time for | 2 | vials* |
| | Reconstitution | | |
| AAM 001-00141 | Determination of the Appearance of | 2 | vials* |
| | Antegren and Matching Placebo | | |
| | Final Container** | | |
| AAM 001-00143 | Determination of pH of Antegren | 1 | mL* |
| | Drug Substance, Final Container and | | |
| | Placebo for Antegren | | |
| AAM 001-00380 | Non-Reduced SDS-Page and | 50 | μL |
| | Reduced SDS-PAGE** | | |
| EOP 001-01198 | Determination of Charge Distribution | 50 | μL |
| | of Antegren Drug Substance and | | |
| | Final Container by Weak Cation | | |
| | Exchange Chromatography** | | |
| EOP 001-01205 | Size Exclusion Chromatography** | 50 | μL |
| AAM 001-00025 | Concentration of Drug Substance, | 200 | μL |
| | Final Container, and Placebo for | | |
| | Antegren by A280** | | |

*Non-destructive, use vial for subsequent testing.
**Modification to method required, see Section 3.3 below.

TABLE 60
Study Methods

| Method Number | Title | Volume Needed |
|---|---|---|
| 160a.213 | VCAM Lysate | 1 vial** |
| 22d.530 | Reducing Gel Chip CE | 1 vial** |
| 22d.596 | Non-reducing Gel Chip CE | 1 vial** |
| 22d.544b and | Methionine Oxidation | 1 vial** |
| 22d.552a | | |
| 22d.84 | Particulates in Small Volume | 1 vial** |
| | Parenterals | |

**One vial used for all 5 assays

TABLE 61
Stability Tests and Time points for Pre-Lyophilized Bulk

| | | 1 | | 2 | | 3 | | 6 | | 12 Month |
|---|---|---|---|---|---|---|---|---|---|---|
| | | Month | | Month | | Month | | Month | | or EOS |
| Test Methods | Initial | 2-8 | 25 | 2-8 | 25 | 2-8 | 25 | 2-8 | 25 | 2-8 |
| Appearance (AAM | X | X | X | X | X | X | X | X | X | X |
| 001-00141) | | | | | | | | | | |
| Protein | X | X | X | X | X | X | X | X | X | X |
| Concentration by | | | | | | | | | | |
| A280 | | | | | | | | | | |
| Size Exclusion | X | X | X | X | X | X | X | X | X | X |
| Chromatography | | | | | | | | | | |
| (SEC) | | | | | | | | | | |
| Cation Exchange | X | X | X | X | X | X | X | X | X | X |
| Chromatography | | | | | | | | | | |
| (CEX) | | | | | | | | | | |
| SDS-PAGE: | X | X | X | X | X | X | X | X | X | X |
| reduced | | | | | | | | | | |
| SDS-PAGE: non- | X | X | X | X | X | X | X | X | X | X |
| reduced | | | | | | | | | | |
| pH | X | X | X | X | X | X | X | X | X | X |
| VCAM Lysate* | X | | | | | X | X | | | X |
| Reducing Gel Chip* | X | | | | | X | X | | | X |
| Non-reducing Gel | X | | | | | X | X | | | X |
| Chip CE* | | | | | | | | | | |
| Methionine | X | | | | | X | X | | | X |
| oxidation* | | | | | | | | | | |
| Number of samples | 1 | 1 | 1 | 1 | 1 | 1 | 1 | 1 | 1 | 1 |
| to pull per time point | | | | | | | | | | |
| per condition at | | | | | | | | | | |
| Elan | | | | | | | | | | |

TABLE 62
Stability Tests and Time points for Lyophilized Vials

| | | | | | | | | | | | 6 | | 12 Month |
|---|---|---|---|---|---|---|---|---|---|---|---|---|---|
| | | 1 Month | | | 2 Month | | | 3 Month | | | Month | | or EOS |
| Test Methods | Initial | 2-8 | 25 | 40 | 2-8 | 25 | 40 | 2-8 | 25 | 40 | 2-8 | 25 | 2-8 |
| Appearance of cake | X | X | X | X | X | X | X | X | X | X | X | X | X |
| Time for reconstitution | X | X | X | X | X | X | X | X | X | X | X | X | X |
| Moisture | X | | | | | | | | | | X | X | X |
| Appearance of | X | X | X | X | X | X | X | X | X | X | X | X | X |
| reconstituted solution | | | | | | | | | | | | | |
| Protein Concentration | X | X | X | X | X | X | X | X | X | X | X | X | X |
| by A280 | | | | | | | | | | | | | |
| Size Exclusion | X | X | X | X | X | X | X | X | X | X | X | X | X |
| Chromatography (SEC) | | | | | | | | | | | | | |
| Cation Exchange | X | X | X | X | X | X | X | X | X | X | X | X | X |
| Chromatography (CEX) | | | | | | | | | | | | | |
| SDS-PAGE: reduced | X | X | X | X | X | X | X | X | X | X | X | X | X |
| SDS-PAGE: non- | X | X | X | X | X | X | X | X | X | X | X | X | X |
| reduced | | | | | | | | | | | | | |
| pH | X | X | X | X | X | X | X | X | X | X | X | X | X |
| VCAM Lysate* | X | | | | | | | | X | X | X | X | X |
| Reducing Gel Chip* | X | | | | | | | | X | X | X | X | X |
| Non-reducing Gel | X | | | | | | | | X | X | X | X | X |
| Chip* | | | | | | | | | | | | | |
| Methionine oxidation* | X | | | | | | | | X | X | X | X | X |
| SVP Particulates* | X | | | | | | | | X | X | X | X | X |
| Number of vials to pull | 2 | 2 | 2 | 2 | 2 | 2 | 2 | 2 | 2 | 2 | 2 | 2 | 2 |
| per time point per | | | | | | | | | | | | | |
| condition at Elan | | | | | | | | | | | | | |

Reconstituted Stability

At the following time points, 4 vials were pulled from 2-8° C., reconstituted and then placed at 2-8° C. and 25° C. for one week to examine the stability of the reconstituted material. For ease of use of analytical equipment and time, these samples were pulled one week before the scheduled timepoint, reconstituted, placed at the desired temperature and then assayed along side the other samples.

TABLE 63
Stability Tests and Time points for Reconstituted Vials

| | | 3 | | | | 12 Month |
|---|---|---|---|---|---|---|
| | | Month | | 6 Month | | or EOS |
| Test Methods | Initial | 2-8 | 25 | 2-8 | 25 | 2-8 |
| Appearance (AAM 001- | X | X | X | X | X | X |
| 00141) | | | | | | |
| Protein Concentration by | X | X | X | X | X | X |
| A280 | | | | | | |
| Size Exclusion | X | X | X | X | X | X |
| Chromatography (SEC) | | | | | | |
| Cation Exchange | X | X | X | X | X | X |
| Chromatography (CEX) | | | | | | |
| SDS-PAGE: reduced | X | X | X | X | X | X |
| SDS-PAGE: non-reduced | X | X | X | X | X | X |
| pH | X | X | X | X | X | X |
| VCAM Lysate* | | X | X | | | X |
| Reducing Gel Chip* | | X | X | | | X |
| Non-reducing Gel Chip CE* | | X | X | | | X |
| Methionine oxidation * | | X | X | | | X |
| Number of samples to pull | 2 | 2 | 2 | 2 | 2 | 2 |
| per time point per condition at | | | | | | |
| Elan | | | | | | |

Note:
Vials will be pulled from 2-8° C. at the listed time points, reconstituted and then stored at either 2-8° C. or 25° C. for one week and assayed as above.

### Data

### APPENDIX A

### Total Number of Vials to be Placed at Each Condition. Note: 2-8° Numbers Include Initial Time Point Testing as Necessary

TABLE 64
Pre-Lyophilized bulk sample requirements

| | # vials required for | | |
|---|---|---|---|
| Temperature | Elan testing | Vial Overage | Total Required |
| 5° C. | 6 | 5 | 11 vials |
| 25° C./60% RH | 4 | 4 | 8 vials |

| | |
|---|---|
| Total 5° C. and 25° C./60% RH studies | 19 vials |

TABLE 65
Lyophilized sample requirements (including for reconstitution)

| | # vials required for | | |
|---|---|---|---|
| Temperature | Elan testing | Vial Overage | Total Required |
| 5° C. | 24 | 12 | 36 vials |
| 25° C./60% RH | 8 | 5 | 13 vials |
| 40° C./75% RH | 6 | 4 | 10 vials |

| | |
|---|---|
| Total 5° C. and 25° C./60% RH studies | 59 vials |

### Material, Methods and Testing Time Points

TABLE 66
Study Methods

| Method Number | Title | Volume Needed | |
|---|---|---|---|
| EOP 000-01291 | Determination of Appearance of Lyophilized Vials | 2 | vials* |
| EOP 000-01290⁺ or EOP | Moisture | 2 | vials |
| 03201299** | | | |
| EOP 000-01292 | Determination of Time for Reconstitution | 2 | vials* |
| AAM 001-00141 | Determination of the Appearance of Antegren and | 2 | vials* |
| | Matching Placebo Final Container** | | |
| AAM 001-00143 | Determination of pH of Antegren Drug Substance, Final | 1 | mL* |
| | Container and Placebo for Antegren | | |
| AAM 001-00380 | Non-Reduced SDS-Page and Reduced SDS-PAGE** | 50 | μL |
| EOP 001-01198 | Determination of Charge Distribution of Antegren Drug | 50 | μL |
| | Substance and Final Container by Weak Cation Exchange | | |
| | Chromatography** | | |
| EOP 001-01205 | Size Exclusion Chromatography** | 50 | μL |
| AAM 001-00025 | Concentration of Drug Substance, Final Container, and | 200 | μL |
| | Placebo for Antegren by A280** | | |

*Non-destructive use vial for subsequent testing.
**Modification to method required, see Section 3.3 below.
⁺Appropriate SOP to be determined, see text.

TABLE 67
Study Methods

| | | Volume |
|---|---|---|
| Method Number | Title | Needed |
| 22d.394⁺ | Karl Fisher | 2 vials |
| 160a.213 | VCAM Lysate | 1 vial** |
| 22d.530 | Reducing Gel Chip CE | 1 vial** |
| 22d.596 | Non-reducing Gel Chip CE | 1 vial** |
| 22d.544b and | Methionine Oxidation | 1 vial** |
| 22d.552a | | |
| 22d.84 | Particulates in Small Volume Parenterals | 1 vial** |

**One vial used for all 5 assays.
⁺Appropriate SOP to be determined, see text.

### Modifications Required to Assays

Size Exclusion chromatography is modified to include that the data collection should be made at the A280 nm wavelength.

Justification: The increased mass on the column from approximately 40 μg to 400 μg necessitates the use of the less sensitive wavelength.

Section 3.5 Table 5: The table will be modified to indicate that a total of 4 vials are necessary for the initial time point testing, at 3 months and at 12 months or end of study.

Justification: This table neglected to take into account the 2 additional vials needed at these time points for moisture testing. This testing is destructive and therefore the contents of the vials cannot be recovered for additional use.

TABLE 68
Stability Tests and Time points for Lyophilized Vials

| | | | | | | | | | | | 6 | | 12 Month |
|---|---|---|---|---|---|---|---|---|---|---|---|---|---|
| | | 1 Month | | | 2 Month | | | 3 Month | | | Month | | or EOS |
| Test Methods | Initial | 2-8 | 25 | 40 | 2-8 | 25 | 40 | 2-8 | 25 | 40 | 2-8 | 25 | 2-8 |
| Appearance of cake | X | X | X | X | X | X | X | X | X | X | X | X | X |
| Time for reconstitution | X | X | X | X | X | X | X | X | X | X | X | X | X |
| Moisture | X | | | | | | | | | | X | X | X |
| Appearance of | X | X | X | X | X | X | X | X | X | X | X | X | X |
| reconstituted solution | | | | | | | | | | | | | |
| Protein Concentration | X | X | X | X | X | X | X | X | X | X | X | X | X |
| by A280 | | | | | | | | | | | | | |
| Size Exclusion | X | X | X | X | X | X | X | X | X | X | X | X | X |
| Chromatography (SEC) | | | | | | | | | | | | | |
| Cation Exchange | X | X | X | X | X | X | X | X | X | X | X | X | X |
| Chromatography (CEX) | | | | | | | | | | | | | |
| SDS-PAGE: reduced | X | X | X | X | X | X | X | X | X | X | X | X | X |
| SDS-PAGE: non- | X | X | X | X | X | X | X | X | X | X | X | X | X |
| reduced | | | | | | | | | | | | | |
| pH | X | X | X | X | X | X | X | X | X | X | X | X | X |
| VCAM Lysate* | X | | | | | | | X | X | X | X | X | X |
| Reducing Gel Chip* | X | | | | | | | X | X | X | X | X | X |
| Non-reducing Gel | X | | | | | | | X | X | X | X | X | X |
| Chip* | | | | | | | | | | | | | |
| Methionine oxidation* | X | | | | | | | X | X | X | X | X | X |
| SVP Particulates* | X | | | | | | | | X | X | X | X | X |
| Number of vials to pull | 4 | 2 | 2 | 2 | 2 | 2 | 2 | 2 | 2 | 2 | 4 | 4 | 4 |
| per time point per | | | | | | | | | | | | | |
| condition at Elan | | | | | | | | | | | | | |

TABLE 69
Natalizumab, Pre-Lyophilized Bulk Stability Results

| | | |
|---|---|---|
| Product: Natalizumab, Lyophilized, 160 mg/vial | Mfr. Bulk Lot No: N/A | Control No: K65001 AQS: 2071 |
| Mfr. Date: Pre-Lyophilized Bulk | Strength: 40 mg/mL | Stability Initiation Date: |
| Storage Conditions: 5° C. ± 3° C. Upright | Description: 5 mL fill in polypropylene vials | Mfr. Lot No: LYO121504 |

Test Performed

| | | | | | | | SDS-PAGE | | | Size Exclusion | | |
|---|---|---|---|---|---|---|---|---|---|---|---|---|
| | | | | Cation Exchange | | | Report value | | | Chromatography | | |
| | | Conc | | Chromatography (CEX) | | | | Non-Reduced | | Report value | | |
| Time | Appearance | Report | pH | Report value | | | | MW | | | % Impurity | |
| point | Report | value | Report | % Lower | % Higher | | Reduced | comparable | % Half | % | % | % |
| (months) | Observation | (mg/mL) | value | Isoforms | Isoforms | % Peak 4 | % IgG | to Ref Std | Antibody | purity | Oligomer | LMW2 |
| 0 | Colorless, | 41 | 6.0 | 17.0 | 13.4 | 69.6 | 99.5 | Conforms | 11.0 | 99.7 | 0.2 | 0.1 |
| | slightly | | | | | | | | | | | |
| | opalescent. | | | | | | | | | | | |
| | Particles: 1 | | | | | | | | | | | |
| 1 | Colorless, | 41 | 6.1 | 15.8 | 14.8 | 69.4 | 99.2 | Conforms | 9.6 | 99.7 | 0.2 | 0.1 |
| | slightly | | | | | | | | | | | |
| | opalescent. | | | | | | | | | | | |
| | Particles: 1 | | | | | | | | | | | |
| 2 | Colorless, | 41 | 6.1 | 17.2 | 12.1 | 70.8 | 99.5 | Conforms | 9.1 | 99.7 | 0.2 | 0.1 |
| | slightly | | | | | | | | | | | |
| | opalescent. | | | | | | | | | | | |
| | Particles: 0 | | | | | | | | | | | |
| 3 | Colorless, | 40 | 6.2 | 18.2 | 12.4 | 69.4 | 99.4 | Conforms | 8.3 | 99.7 | 0.2 | 0.1 |
| | slightly | | | | | | | | | | | |
| | opalescent. | | | | | | | | | | | |
| | Particles: 0 | | | | | | | | | | | |
| 6 | Colorless, | 41 | 6.1 | 16.9 | 14.2 | 68.9 | 99.4 | Conforms | 8.3 | 99.7 | 0.3 | 0.0 |
| | slightly | | | | | | | | | | | |
| | opalescent. | | | | | | | | | | | |
| | Particles: 0 | | | | | | | | | | | |
| 12 | Colorless, | 41 | 6.1 | 17.9 | 11.9 | 70.2 | 100.0 | Conforms | 9.7 | 99.7 | 0.3 | 0.1 |
| | slightly | | | | | | | | | | | |
| | opalescent. | | | | | | | | | | | |
| | Particles: 0 | | | | | | | | | | | |

TABLE 70
Natalizumab, Pre-Lyophilized Bulk Stability Results

| | | |
|---|---|---|
| Product: Natalizumab, Lyophilized, 160 mg/vial | Mfr. Bulk Lot No: N/A | Control No: K65001 AQS: 2071 |
| Mfr. Date: Pre-Lyophilized Bulk | Strength: 40 mg/mL | Stability Initiation Date: |
| Storage Conditions: 25° C. ± 2° C./60% RH + 5% RH | Description: 5 mL fill in polypropylene vials | Mfr. Lot No: LYO121504 |
| Upright | | |

Test Performed

| | | | | | | | SDS-PAGE | | | Size Exclusion | | |
|---|---|---|---|---|---|---|---|---|---|---|---|---|
| | | | | Cation Exchange | | | Report value | | | Chromatography | | |
| | | Conc | | Chromatography (CEX) | | | | Non-Reduced | | Report value | | |
| Time | Appearance | Report | pH | Report value | | | | MW | | | % Impurity | |
| point | Report | value | Report | % Lower | % Higher | | Reduced | comparable | % Half | % | % | % |
| (months) | Observation | (mg/mL) | value | Isoforms | Isoforms | % Peak 4 | % IgG | to Ref Std | Antibody | purity | Oligomer | LMW2 |
| 0 | Colorless, | 41 | 6.0 | 17.0 | 13.4 | 69.9 | 99.5 | Conforms | 11.0 | 99.7 | 0.2 | 0.1 |
| | slightly | | | | | | | | | | | |
| | opalescent. | | | | | | | | | | | |
| | Particles: 1 | | | | | | | | | | | |
| 1 | Colorless, | 41 | 6.1 | 17.3 | 14.1 | 68.6 | 99.1 | Conforms | 9.1 | 99.6 | 0.3 | 0.1 |
| | slightly | | | | | | | | | | | |
| | opalescent. | | | | | | | | | | | |
| | Particles: 1 | | | | | | | | | | | |
| 2 | Colorless, | 41 | 6.1 | 20.5 | 10.4 | 69.2 | 99.5 | Conforms | 9.9 | 99.6 | 0.3 | 0.1 |
| | slightly | | | | | | | | | | | |
| | opalescent. | | | | | | | | | | | |
| | Particles: 0 | | | | | | | | | | | |
| 3 | Colorless, | 41 | 6.1 | 23.6 | 10.2 | 66.2 | 99.1 | Conforms | 8.6 | 99.6 | 0.3 | 0.1 |
| | slightly | | | | | | | | | | | |
| | opalescent. | | | | | | | | | | | |
| | Particles: 0 | | | | | | | | | | | |
| 6 | Colorless, | 42 | 6.1 | 27.7 | 11.3 | 61.0 | 98.8 | Conforms | 8.6 | 99.6 | 0.4 | |
| | slightly | | | | | | | | | | | |
| | opalescent. | | | | | | | | | | | |
| | Particles: 0 | | | | | | | | | | | |

TABLE 71
Natalizumab, Lyophilized Stability Results

| | | |
|---|---|---|
| Product: Natalizumab, | Mfr. Bulk Lot No: N/A | Control No: K65001 AQS: 2071 |
| Lyophilized, 160 mg/vial | Strength: 120 mg/mL | Stability Initiation Date: |
| Mfr. Date: | Description: 5 mL vials containing lyophilized cake with | Mfr. Lot No: LYO121504 |
| Storage Conditions: 5° C. ± 3° C. | butyl rubber stopper and Flip-off cap. | |
| Upright | Vials to be reconstituted with 1 mL | |
| | of WFI or other purified water | |

Test Performed

| | | | | | | | CationExchang | | |
|---|---|---|---|---|---|---|---|---|---|
| | | | | | | Time of | Chromatography(CEX) | | |
| | Appearance | | Conc. | Moisture | pH | Reconstitution | Report value | | |
| Time point | Report Observation | | Report value | Report | Report | Time of | % Lower | % Higher | |
| (months) | Cake | Solution | (mg/mL) | value | value | Reconstitution | Isoforms | Isoforms | % Peak 4 |
| 0 | White 1 | Colorless, | 136 | 0.3% | 6.0 | 11 m 29 s | 17.2 | 13.4 | 69.4 |
| | Cake | slightly | | | | 7 m 38 s | | | |
| | | opalescent. | | | | | | | |
| | | Particles | | | | | | | |
| 1 | White 1 | Colorless, | 127 | NR | 6.1 | 7 m 18 s | 16.1 | 14.5 | 69.4 |
| | Cake | slightly | | | | 6 m 41 s | | | |
| | | opalescent. | | | | | | | |
| | | Particles: | | | | | | | |
| 2 | White 1 | Colorless, | 135 | NR | 6.1 | 6 m 47 s | 17.2 | 12.3 | 70.5 |
| | Cake | slightly | | | | 7 m 08 s | | | |
| | | opalescent. | | | | | | | |
| | | Particles: | | | | | | | |
| 3 | White 1 | Colorless, | 127 | NR | 6.2 | 5 m 22 s | 17.8 | 12.7 | 69.5 |
| | Cake1 | slightly | | | | 4 m 16 s | | | |
| | | opalescent. | | | | | | | |
| | | Particles | | | | | | | |
| | | 1 and 0 | | | | | | | |
| 6 | White 1 | Colorless, | 126 | 0.2 | 6.1 | 4 m 57 s | 15.9 | 15.2 | 68.8 |
| | Cake | slightly | | | | 4 m 40 s | | | |
| | | opalescent. | | | | 4 m 15 s | | | |
| | | Particles: | | | | | | | |
| | | 0, 1, 0, 03 | | | | | | | |
| 12 | White 1 | Colorless, | 132 | 0.8 | 6.2 | 6 m 36 s | 16.9 | 12.6 | 70.5 |
| | Cake | slightly | | | | 8 m 17 s | | | |
| | | opalescent. | | | | | | | |
| | | Particles: | | | | | | | |
| | | 0, 0 | | | | | | | |

| | | | | Size Exclusion | | |
|---|---|---|---|---|---|---|
| | SDS-PAGE | | | Chromatography | | |
| | Report Value | | | Report Value | | |
| | | Non-Reduced | | | % Impurity | |
| Time point | Reduced | MW comparable | % Half | % | % | % |
| (months) | % IgG | to Ref Std | Antibody | Purity | Oligomer | LMW2 |
| 0 | 99.52 | Conform | 10.7 | 99.6 | 0.3 | 0.1 |
| 1 | 99.9 | Conform | 8.1 | 99.5 | 0.3 | 0.1 |
| 2 | 99.5 | Conform | 10.6 | 99.5 | 0.3 | 0.1 |
| 3 | 99.7 | Conform | 9.0 | 99.3 | 0.3 | 0.3 |
| 6 | 99.7 | Conform | 7.8 | 99.5 | 0.4 | 0.1 |
| 12 | 100.0 | Conform | 8.5 | 99.5 | 0.4 | 0.1 |

1Refer to Analytical Summary for more detail description.
2 The initial OOT result was determined to be laboratory error (see LIR# 05-007 for more information.)
NR = test not required at time point according to protocol.
3 See QIR# G1-05-033.

TABLE 72
Natalizumab, Lyophilized Stability Results

| | | |
|---|---|---|
| Product: Natalizumab, | Mfr. Bulk Lot No: N/A | Control No: K65001 AQS: 2071 |
| Lyophilized, 160 mg/vial | Strength: 120 mg/mL | Stability Initiation Date: |
| Mfr. Date: | Description: 5 mL vial containing lyophilized | Mfr. Lot No: LYO121504 |
| Storage Conditions: 25° C. ± 2° C./60% | cake with butyl rubber stopper and Flip-off cap. | |
| RH ± 5% RH Upright | Vials to be reconstituted with 1 mL of WFI or other | |
| | purified water | |

Test Performed

| | | | | | | | Caution Exchange | | |
|---|---|---|---|---|---|---|---|---|---|
| | | | | | | Time of | Chromatography(CEX) | | |
| | Appearance | | Conc. | Moisture | pH | Reconstitution | Report value | | |
| Time point | Report Observation | | Report value | Report | Report | Time of | % Lower | % Higher | |
| (months) | Cake | Solution | (mg/mL) | value | value | Reconstitution | Isoforms | Isoforms | % Peak 4 |
| 0 | White 1 | Colorless, | 136 | 0.3% | 6.01 | 11 m 29 s | 17.2 | 13.4 | 69.4 |
| | Cake | slightly | | | | 7 m 38 s | | | |
| | | opalescent. | | | | | | | |
| | | Particles | | | | | | | |
| | | 1′ | | | | | | | |
| 1 | White 1 | Colorless, | 126 | NR | 6.1 | 7 m 18 s | 16.1 | 14.7 | 69.2 |
| | Cake | slightly | | | | 6 m 41 s | | | |
| | | opalescent. | | | | | | | |
| | | Particles: 1 | | | | | | | |
| 2 | White 1 | Colorless, | 140 | NR | 6.1 | 6 m 32 s | 17.0 | 12.5 | 70.6 |
| | Cake | slightly | | | | 6 m 09 s | | | |
| | | opalescent. | | | | | | | |
| | | Particles: 1 | | | | | | | |
| 3 | White 1 | Colorless, | 117 | NR | 6.2 | 6 m 17 s | 17.9 | 12.6 | 69.5 |
| | Cake1 | slightly | | | | 5 m 41 s | | | |
| | | opalescent. | | | | | | | |
| | | Particles 0 | | | | | | | |
| 6 | White 1 | Colorless, | 135 | 0.5 | 6.1 | 4 m 37 s | 16.7 | 15.0 | 68.3 |
| | Cake | slightly | | | | 4 m 45 s | | | |
| | | opalescent. | | | | 6 m 27 s³ | | | |
| | | Particles: | | | | | | | |
| | | 0, 0, 1, 0³ | | | | | | | |

| | | | | Size Exclusion | | |
|---|---|---|---|---|---|---|
| | SDS-PAGE | | | Chromatography | | |
| | Report Value | | | Report Value | | |
| | | Non-Reduced | | | % Impurity | |
| Time point | Reduced | MW comparable | % Half | % | % | % |
| (months) | % IgG | to Ref Std | Antibody | Purity | Oligomer | LMW2 |
| 0 | 99.52² | Conform² | 10.7 | 99.6 | 0.3 | 0.1 |
| 1 | 99.8 | Conform | 9.1 | 99.3 | 0.6 | 0.1 |
| 2 | 99.5 | Conform | 10.3 | 99.2 | 0.7 | 0.1 |
| 3 | 99.7 | Conform | 8.2 | 99.1 | 0.8 | |
| 6 | 99.6 | Conform | 8.4 | 98.8 | 1.0 | 0.1 |

1Refer to Analytical Summary for more detail description.
²The initial OOT result was determined to be laboratory error (see LIR# 05-007 for more information.)
NR = test not required at time point according to protocol.
³See QIR# G1-05-033.

TABLE 73
Natalizumab, Lyophilized Stability Results

| | | |
|---|---|---|
| Product: Natalizumab, Lyophilized, | Mfr. Bulk Lot No: N/A | Control No: K65001 AQS: 2071 |
| 160 mg/vial | Strength: 120 mg/mL | Stability Initiation Date: |
| Mfr. Date: | Description: 5 mL vial containing lyophilized | Mfr. Lot No: LYO121504 |
| Storage Conditions: 40° C. ± 2° C./75% | cake with butyl rubber stopper and Flip-off cap. | |
| RH ± 5% RH Upright | Vials to be reconstituted with 1 mL of WFI or other | |
| | purified water | |

Test Performed

| | | | | | | | Caution Exchange | | |
|---|---|---|---|---|---|---|---|---|---|
| | | | | | | Time of | Chromatography(CEX) | | |
| | Appearance | | Conc. | Moisture | pH | Reconstitution | Report value | | |
| Time point | Report Observation | | Report value | Report | Report | Time of | % Lower | % Higher | |
| (months) | Cake | Solution | (mg/mL) | value | value | Reconstitution | Isoforms | Isoforms | % Peak 4 |
| 0 | White 1 | Colorless, | 136 | 0.3% | 6.0 | 11 m 29 s | 17.2 | 13.4 | 69.4 |
| | Cake | slightly | | | | 7 m 38 s | | | |
| | | opalescent. | | | | | | | |
| | | Particles | | | | | | | |
| | | 1′ | | | | | | | |
| 1 | White 1 | Colorless, | 125 | NR | 6.1 | 5 m 30 s | 16.2 | 15.0 | 68.8 |
| | Cake | slightly | | | | 4 m 40 s | | | |
| | | opalescent. | | | | | | | |
| | | Particles: 1 | | | | | | | |
| 2 | White 1 | Colorless, | 131 | NR | 6.1 | 5 m 40 s | 17.2 | 13.2 | 69.5 |
| | Cake | slightly | | | | 5 m 38 s | | | |
| | | opalescent. | | | | | | | |
| | | Particles: 1 | | | | | | | |
| 3 | White 1 | Colorless, | 129 | NR | 6.2 | 4 m 38 s | 18.4 | 12.9 | 68.6 |
| | Cake1 | slightly | | | | 6 m 30 s | | | |
| | | opalescent. | | | | | | | |
| | | Particles 0 | | | | | | | |

| | | | | Size Exclusion | | |
|---|---|---|---|---|---|---|
| | SDS-PAGE | | | Chromatography | | |
| | Report Value | | | Report Value | | |
| | | Non-Reduced | | | % Impurity | |
| Time point | Reduced | MW comparable | % Half | % | % | % |
| (months) | % IgG | to Ref Std | Antibody | Purity | Oligomer | LMW2 |
| 0 | 99.5² | Conform² | 10.7 | 99.6 | 0.3 | 0.1 |
| 1 | 99.6 | Conform | 9.6 | 98.7 | 1.2 | 0.1 |
| 2 | 99.5 | Conform | 9.8 | 98.3 | 1.6 | 0.2 |
| 3 | 99.4 | Conform | 8.2 | 97.7 | 2.0 | |

1Refer to Analytical Summary for more detail description.
²The initial OOT result was determined to be laboratory error (see LIR# 05-007 for more information.
NR = test not required at time point according to protocol.

TABLE 74
Natalizumab, Reconstituted Vials Stability Results

| | | |
|---|---|---|
| Product: Natalizumab, | Mfr. Bulk Lot No: N/A | Control No: K65001 AQS: 2071 |
| Lyophilized, 160 mg/vial | Strength: 120 mg/mL | Stability Initiation Date: |
| Reconstituted Vials | Description: 5 mL, type I glass vial | Mfr. Lot No: LYO121504 |
| Mfr. Date: | | |
| Storage Conditions: 5° C. ± 3° C. Upright | | |

Test Performed

| | | | | | | Caution Exchange | | |
|---|---|---|---|---|---|---|---|---|
| | | | | | Time of | Chromatography(CEX) | | |
| | Appearance | | Conc. | pH | Reconstitution | Report value | | |
| Time point | Report Observation | | Report value | Report | Time of | % Lower | % Higher | |
| (months) | Cake | Solution | (mg/mL) | value | Reconstitution | Isoforms | Isoforms | % Peak 4 |
| 0 | White 1 | Colorless, | 132 | 6.0 | NR | 16.9 | 13.7 | 69.4 |
| | Cake | slightly | | | | | | |
| | | opalescent. | | | | | | |
| | | Particles | | | | | | |
| | | 1′ | | | | | | |
| 3 | White 1 | Colorless, | 134 | 6.1 | 5 m 17 s | 18.1 | 12.4 | 69.5 |
| | Cake | slightly | | | 4 m 27 s | | | |
| | | opalescent. | | | | | | |
| | | Particles: 0 | | | | | | |
| 6 | White 1 | Colorless, | 133 | 6.1 | 4 m 13 s | 16.9 | 14.1 | 69.0 |
| | Cake | slightly | | | 6 m 03 s | | | |
| | | opalescent. | | | | | | |
| | | Particles: | | | | | | |
| | | 0, 1 | | | | | | |
| 12 | White 1 | Colorless, | 137 | 6.2 | 5 m 59 s | 17.1 | 12.5 | 70.5 |
| | Cake1 | slightly | | | 6 m 18 s | | | |
| | | opalescent. | | | | | | |
| | | Particles | | | | | | |
| | | 0, 1 | | | | | | |

| | | | | Size Exclusion | | |
|---|---|---|---|---|---|---|
| | SDS-PAGE | | | Chromatography | | |
| | Report Value | | | Report Value | | |
| | | Non-Reduced | | | % Impurity | |
| Time point | Reduced | MW comparable | % Half | % | % | |
| (months) | % IgG | to Ref Std | Antibody | Purity | Oligomer | % LMW2 |
| 0 | 99.5 | Conform | 11.5 | 99.6 | 0.3 | 0.1 |
| 3 | 99.7 | Conform | 8.1 | 99.3 | 0.3 | 0.4 |
| 6 | 99.7 | Conform | 8.4 | 99.5 | 0.4 | 0.1 |
| 12 | 100.0 | Conform | 10.5 | 99.5 | 0.5 | |

1Refer to Analytical Summary for more detail description
NR = test not required at time point according to protocol.

TABLE 75
Natalizumab, Reconstituted Vials Stability Results

| | | |
|---|---|---|
| Product: Natalizumab, | Mfr. Bulk Lot No: N/A | Control No: K65001 AQS: 2071 |
| Lyophilized, 160 mg/vial | Strength: 120 mg/mL | Stability Initiation Date: |
| Reconstituted Vials | Description: 5 mL, type I glass vial | Mfr. Lot No: LYO121504 |
| Mfr. Date: | | |
| Storage Conditions: 25° C. ± 2° C./60% | | |
| RH ± 5% RH Upright | | |

Test Performed

| | | | | | | Caution Exchange | | |
|---|---|---|---|---|---|---|---|---|
| | | | | | Time of | Chromatography(CEX) | | |
| | Appearance | | Conc. | pH | Reconstitution | Report value | | |
| Time point | Report Observation | | Report value | Report | Time of | % Lower | % Higher | |
| (months) | Cake | Solution | (mg/mL) | value | Reconstitution | Isoforms | Isoforms | % Peak 4 |
| 0 | NR | Colorless, | 132 | 6.0 | NR | 16.9 | 13.7 | 69.4 |
| | | slightly | | | | | | |
| | | opalescent. | | | | | | |
| | | Particles | | | | | | |
| | | 1′ | | | | | | |
| 3 | White 1 | Colorless, | 141 | 6.1 | 7 m 16 s | 18.7 | 11.9 | 69.5 |
| | Cake | slightly | | | 5 m 15 s | | | |
| | | opalescent. | | | | | | |
| | | Particles: 0 | | | | | | |
| 6 | White 1 | Colorless, | 137 | 6.1 | 4 m 12 s | 17.4 | 13.9 | 69.5 |
| | Cake | slightly | | | 5 m 57 s | | | |
| | | opalescent. | | | | | | |
| | | Particles: | | | | | | |
| | | 0, 1 | | | | | | |

| | | | | Size Exclusion | | |
|---|---|---|---|---|---|---|
| | SDS-PAGE | | | Chromatography | | |
| | Report Value | | | Report Value | | |
| | | Non-Reduced | | | % Impurity | |
| Time point | Reduced | MW comparable | % Half | % | % | |
| (months) | % IgG | to Ref Std | Antibody | Purity | Oligomer | % LMW2 |
| 0 | 99.5 | Conform | 11.5 | 99.6 | 0.3 | 0.1 |
| 3 | 99.5 | Conform | 7.9 | 99.2 | 0.4 | 0.4 |
| 6 | 99.5 | Conform | 7.9 | 99.2 | 0.4 | 0.1 |

1Refer to Analytical Summary for more detail description.
NR = test not required at time point according to protocol

1. A stable lyophilized formulation prepared by lyophilizing an aqueous formulation, wherein the aqueous formulation comprises:
(a) about 20 mg/ml to about 80 mg/ml natalizumab;
(b) a buffer having a pH of about 5.5 to about 6.5;
(c) about 20 mg/ml to about 80 mg/ml sucrose; and
(d) about 0.02 to about 0.08% polysorbate.

2. The stable lyophilized formulation of claim 1, wherein said aqueous formulation comprises about 30 mg/ml to about 80 mg/ml natalizumab.

3. The stable lyophilized formulation of claim 2, wherein said aqueous formulation comprises about 40 mg/ml natalizumab.

4. The stable lyophilized formulation of claim 1, wherein said buffer has a pH of about 6.0.

5. The stable lyophilized formulation of claim 1, wherein said buffer is histidine.

6. The stable lyophilized formulation of claim 5, wherein said histidine is present in said aqueous formulation at a concentration of about 1 mM to about 12 mM.

7. The stable lyophilized formulation of claim 6, wherein said histidine is present in said aqueous formulation at a concentration of about 6 mM.

8. The stable lyophilized formulation of claim 1, wherein said sucrose is present in said aqueous formulation at a concentration of about 20 mg/ml to about 80 mg/ml.

9. The stable lyophilized formulation of claim 8, wherein said sucrose is present in said aqueous formulation at a concentration of about 41 mg/ml.

10. The stable lyophilized formulation of claim 1, wherein said polysorbate is polysorbate 80.

11. The stable lyophilized formulation of claim 10, wherein said polysorbate 80 is present in said aqueous formulation at a concentration of about 0.02% to about 0.08%.

12. The stable lyophilized formulation of claim 11, wherein said polysorbate 80 is present in said aqueous formulation at a concentration of about 0.04%.

13. The stable lyophilized formulation of claim 1, wherein the weight ratio of natalizumab to sucrose in said aqueous formulation is about 0.5:1 to about 2:1.

14. The stable lyophilized formulation of claim 13, wherein the weight ratio of natalizumab to sucrose in said aqueous formulation is about 1:1.

15. The stable lyophilized formulation of claim 1, wherein the molar ratio of sucrose to natalizumab in said aqueous formulation is about 300:1 to about 500:1.

16. The stable lyophilized formulation of claim 15, wherein the molar ratio of sucrose to natalizumab in said aqueous formulation is about 400:1 to about 500:1.

17. The stable lyophilized formulation of claim 16, wherein the molar ratio of sucrose to natalizumab in said aqueous formulation is about 450:1.

18. The stable lyophilized formulation of claim 1, wherein said aqueous formulation further comprises a bulking agent.

19. The stable lyophilized formulation of claim 1, wherein said aqueous formulation further comprises a tonicity modifier.

20. The stable lyophilized formulation of claim 1, wherein said aqueous formulation comprises:
(a) about 40 mg/ml natalizumab;
(b) about 6 mM histidine, pH about 6.0;
(c) about 41 mg/ml sucrose; and
(d) about 0.04% polysorbate 80.

21. A stable reconstituted formulation comprising:
(i) about 80 to about 160 mg/ml natalizumab;
(ii) about 18 mM histidine at a pH of about 6.0;
(iii) about 123 mg/ml sucrose; and
(iv) about 0.12% polysorbate 80; wherein:
the reconstituted formulation has been prepared from a stable lyophilized formulation prepared by lyophilizing an aqueous formulation comprising:
(a) about 40 mg/ml natalizumab;
(b) about 6 mM histidine, pH about 6.0;
(c) about 41 mg/ml sucrose; and
(d) about 0.04% polysorbate 80.

22. The stable reconstituted formulation of claim 21, wherein said stable reconstituted formulation comprises about 120 mg/ml natalizumab.

23. A method for preparing a stable reconstituted formulation, comprising reconstitution of a lyophilized formulation that has been prepared by lyophilizing an aqueous formulation comprising:
(a) about 30 to about 60 mg/ml natalizumab;
(b) a buffer having a pH of about 5.5 to about 6.5;
(c) about 20 to about 50 mg/ml sucrose; and
(d) about 0.02 to about 0.08% polysorbate.

24. The method of claim 24, wherein said aqueous formulation comprises about 40 mg/ml to about 50 mg/ml natalizumab.

25. The method of claim 25, wherein said aqueous formulation comprises about 40 mg/ml natalizumab.

26. The method of claim 23, wherein said buffer has a pH of about 6.0.

27. The method of claim 23, wherein said buffer is histidine.

28. The method of claim 28, wherein said histidine is present in said aqueous formulation at a concentration of about 1 mM to about 12 mM.

29. The method of claim 29, wherein said histidine is present in said aqueous formulation at a concentration of about 6 mM.

30. The method of claim 23, wherein said sucrose is present in said aqueous formulation at a concentration of about 20 mg/ml to about 50 mg/ml.

31. The stable lyophilized formulation of claim 31, wherein said sucrose is present in said aqueous formulation at a concentration of about 40 mg/ml.

32. The method of claim 23, wherein said polysorbate is polysorbate 80.

33. The method of claim 32, wherein said polysorbate 80 is present in said aqueous formulation at a concentration of about 0.02% to about 0.08%.

34. The method of claim 33, wherein said polysorbate 80 is present in said aqueous formulation at a concentration of about 0.04%.

35. The method of claim 23, wherein the weight ratio of natalizumab to sucrose in said aqueous formulation is about 0.5:1 to about 2:1.

36. The method of claim 35, wherein the weight ratio of natalizumab to sucrose in said aqueous formulation is about 1:1.

37. The method of claim 23, wherein the molar ratio of sucrose to natalizumab in said aqueous formulation is about 300:1 to about 500:1.

38. The method of claim 37, wherein the molar ratio of sucrose to natalizumab in said aqueous formulation is about 400:1 to about 500:1.

39. The method of claim 38, wherein the molar ratio of sucrose to natalizumab in said aqueous formulation is about 450:1.

40. The method of claim 23, wherein said aqueous formulation further comprises a bulking agent.

41. The method of claim 23, wherein said aqueous formulation further comprises a tonicity modifier.

42. The method of claim 23, wherein said aqueous formulation comprises:
(a) about 40 mg/ml natalizumab;
(b) about 6 mM histidine, pH about 6.0;
(c) about 41 mg/ml sucrose; and
(d) about 0.04% polysorbate 80.

43. A method for treating a subject, comprising administering to said subject a therapeutically effective amount of a stable reconstituted formulation according to claim 23, wherein said subject has a disorder that can be treated by administering natalizumab.
